(19) United States
(12) Patent Application Publication
(10) Pub. No.: US 2024/0390301 A1
(43) Pub. Date: Nov. 28, 2024

(54) COMBINATIONS OF PERIPHERAL 5-HT2A RECEPTOR ANTAGONISTS AND CENTRAL 5-HT2A RECEPTOR AGONISTS

(71) Applicant: GILGAMESH PHARMACEUTICALS, INC., New York, NY (US)

(72) Inventor: Andrew Carry KRUEGEL, Secaucus, NJ (US)

(73) Assignee: GILGAMESH PHARMACEUTICALS, INC., New York, NY (US)

(21) Appl. No.: 18/686,059

(22) PCT Filed: Aug. 23, 2022
(86) PCT No.: PCT/US2022/041274

§ 371 Date: Feb. 23, 2024

Related U.S. Application Data
(60) Provisional application No. 63/235,946, filed on Aug. 23, 2021.

(51) Int. Cl. A61K 31/155; A61K 31/137; A61K 31/4468

(52) U.S. Cl. CPC A61K 31/155; A61K 31/137; A61K 31/4468

(57) ABSTRACT

Methods of treating mood disorders with compounds disclosed herein. Also provided are pharmaceutical compositions that include those compounds.

### BACKGROUND

Agonists of the 5-HT2A receptor are of interest as treatments for various psychiatric disorders, including major depressive disorder. anxiety disorders, and substance use disorders. These compounds include classical psychedelic compounds of the tryptamine, ergoline, phenethylamine, and amphetamine scaffolds, for example, psilocybin and lysergic acid diethylamide. Such substances act on 5-HT2A and related receptors in the central nervous system to induce both profound hallucinogenic effects as well as therapeutic benefits in subjects. However, serotonin receptors of various types, including 5-HT2A, are also expressed in peripheral tissues. Activation of these peripheral serotonin receptors leads in some cases to unpleasant and potentially dangerous peripheral side effects in subjects treated with classical psychedelics, such as vasoconstriction, arterial vasospasm, hypertension, myocardial infarction, coronary spasms, bowel ischemia, peripheral ischemia, changes in platelet aggregation, nausea/vomiting, stomach pain, headaches, such as migraines, and/or feverish sensations. Further, peripheral activation of the 5-HT2B receptor, which is an off target of many 5-HT2A agonists, is associated with induction of valvular heart disease. Accordingly, a method of attenuating these peripheral serotonergic side effects of psychedelics while retaining their centrally mediated therapeutic effects would be highly desirable.

### SUMMARY

The present disclosure includes, among other things, methods of treating a psychiatric disorder in a patient in need thereof. In some embodiments, a method of treating a psychiatric disorder comprises administering to a patient in need thereof a therapeutically effective amount of a combination of a peripheral serotonin receptor antagonist and a 5-HT2A receptor agonist. In an embodiment, the method comprises treating a psychiatric disorder in a patent in need thereof comprising administering to said patient a combination of a peripheral serotonin receptor antagonist selected from the group consisting of xylamidine and BW501C67 and a central 5-HT2A receptor agonist. In an embodiment, the method comprises treating a psychiatric disorder in a patent in need thereof comprising administering to said patient an effective amount of a peripheral serotonin receptor antagonist and further comprising administering to the patient an effective amount of a 5-HT2A receptor agonist. In an embodiment, the method comprises treating a psychiatric disorder in a patent in need thereof comprising administering to said patient an effective amount of a peripheral serotonin receptor antagonist selected from the group consisting of xylamidine and BW501C67 and further comprising administering to the patient an effective amount of a central 5-HT2A receptor agonist.

### BRIEF DESCRIPTION OF THE DRAWINGS

Objects, features, and advantages of the present disclosure will become more clearly apparent when the following description is taken in conjunction with the accompanying drawings, wherein like reference numerals denote like parts and in which:

FIG. 1 depicts graphically in a bar graph the effect of xylamidine pre-treatment on the HTR induced by DOPR in the rat, as discussed in Example 6.

FIG. 2 depicts graphically in a bar graph the effect of BW501C67 pre-treatment on the HTR induced by DOPR in the rat, as discussed in Example 6.

FIG. 3 depicts graphically in a bar graph the effect of Compound 14a pre-treatment on the HTR induced by DOPR in the rat, as discussed in Example 6.

FIG. 4 depicts graphically in a bar graph the effect of sarpogrelate pre-treatment on the HTR induced by DOPR in the rat, as discussed in Example 6.

FIG. 5 graphically depicts in a bar graph the effect of xylamidine pre-treatment on DOPR-induced elevation of MABP in the rat at 10-30 min after antagonist administration, as discussed in Example 7.

FIG. 6 depicts graphically in a bar graph the effect of BW501C67 pre-treatment on DOPR-induced elevation of MABP in the rat at 10-30 min after antagonist administration, as discussed in Example 7.

FIG. 7 depicts graphically in a bar graph the effect of xylamidine pre-treatment on DOPR-induced elevation of MABP in the rat at 55-65 min after antagonist administration, as discussed in Example 7.

FIG. 8 depicts graphically in a bar graph the effect of BW501C67 pre-treatment on DOPR-induced elevation of MABP in the rat at 55-65 min after antagonist administration, as discussed in Example 7.

FIG. 9 depicts graphically in a bar graph the effect of Compound 14a pre-treatment on DOPR-induced elevation of MABP in the rat at 10-30 min after antagonist administration, as discussed in Example 7.

### DETAILED DESCRIPTION

The present disclosure provides combinations of peripheral serotonin receptor antagonists, such as sarpogrelate, xylamidine, or BW501C67, and the like and various psychedelic compounds acting as agonists of the 5-HT2A receptor. Sarpogrelate is a peripherally restricted and modestly selective 5-HT2A receptor antagonist that also possesses antagonist activity at the 5-HT2C and 5-HT2B receptors. Its major metabolite, sarpogrelate M1, also possesses substantial serotonin receptor antagonist activity. Accordingly, administration of sarpogrelate, sarpogrelate M1, or another peripheral serotonin receptor antagonist in combination with a 5-HT2A receptor agonist, such as a classical psychedelic, blocks the peripheral side effects of the agonist while leaving its therapeutic actions on the central nervous system intact. The two drugs (5-HT2A receptor agonist+sarpogrelate, sarpogrelate M1, or other peripheral serotonin receptor antagonist) may be administered separately or combined as a fixed-dose combination in order the achieve this synergistic effect with a single drug product. Blockade of peripheral 5-HT2A receptor and other serotonin receptor signaling with such a combination leads to a more pleasant and safer therapeutic experience for the subject.

### Peripheral Serotonin Receptor Antagonists

In some embodiments, a peripheral serotonin receptor antagonist is selected from the group consisting of sarpogrelate, temanogrel, xylamidine, BW501C67, BW204C67, a xylamidine analog, a BW501C67 analog and naftidrofuryl. In some embodiments, a peripheral serotonin receptor antagonist is sarpogrelate. In some embodiments, a peripheral serotonin receptor antagonist is temanogrel. In some embodiments, a peripheral serotonin receptor antagonist is xylamidine. In some embodiments, a peripheral serotonin receptor antagonist is BW501C67. In some embodiments, a peripheral serotonin receptor antagonist is BW204C67. In some embodiments, a peripheral serotonin receptor antagonist is a xylamidine analog. In some embodiments, a peripheral serotonin receptor antagonist is a BW501C67 analog. In some embodiments, a peripheral serotonin receptor antagonist is naftidrofuryl. The peripheral serotonin receptor antagonist is present in a therapeutic amount to reduce the peripheral side effects of a 5-HT2A receptor agonist with which the antagonist is administered. In an embodiment, the antagonist is present in an amount that substantially attenuates or completely eliminates the peripheral side effects of a typical fully psychedelic dose of the 5-HT2A receptor agonist with which it is administered to the patient. In an embodiment, the antagonist is present in an amount that reduces the probability that a patient will experience any of the following resulting from the administration of the 5-HT2A receptor agonist: vasoconstriction, arterial vasospasm, hypertension, myocardial infarction, coronary spasms, bowel ischemia, peripheral ischemia, changes in platelet aggregation, nausea/vomiting, stomach pain, feverish sensations, headaches, such as migraine headaches, or any other peripherally mediated side effect of the agonist.

In some embodiments, a peripheral serotonin receptor antagonist is sarpogrelate. In some embodiments, sarpogrelate is administered at a dose between 20 and 200 mg. In some embodiments, sarpogrelate is administered at a dose selected from the group consisting of about 25 mg, about 50 mg, about 100 mg, about 150 mg, and about 200 mg. In some embodiments, sarpogrelate is administered at a dose of about 25 mg. In some embodiments, sarpogrelate is administered at a dose of about 50 mg. In some embodiments, sarpogrelate is administered at a dose of about 100 mg. In some embodiments, sarpogrelate is administered at a dose of about 150 mg. In some embodiments, sarpogrelate is administered at a dose of about 200 mg. In some embodiments, sarpogrelate is administered in a total daily dose of about 300 mg. In some embodiments, sarpogrelate is administered in a dose of about 100 mg t.i.d. In some embodiments, sarpogrelate is administered once daily. In some embodiments. sarpogrelate is administered twice daily. In some embodiments, sarpogrelate is administered three times daily. In some embodiments, sarpogrelate is administered as a controlled-release formulation. In some embodiments, sarpogrelate is administered orally. In some embodiments, sarpogrelate is administered parenterally.

In some embodiments, a peripheral serotonin receptor antagonist is temanogrel. In some embodiments, temanogrel is administered at a dose between 10 and 320 mg. In some embodiments, temanogrel is administered at a dose selected from the group consisting of about 10 mg. In some embodiments, temanogrel is administered at a dose of about 20 mg. In some embodiments, temanogrel is administered at a dose of about 40 mg. In some embodiments, temanogrel is administered at a dose of about 60 mg. In some embodiments, temanogrel is administered at a dose of about 80 mg. In some embodiments, temanogrel is administered at a dose of about 120 mg. In some embodiments, temanogrel is administered at a dose of about 160 mg. In some embodiments, temanogrel is administered at a dose of about 240 mg. In some embodiments, temanogrel is administered at a dose of about 320 mg. In some embodiments, temanogrel is administered once daily. In some embodiments, temanogrel is administered twice daily. In some embodiments, temanogrel is administered three times daily.

In an embodiment, the peripheral serotonin receptor antagonist is xylamidine.

In another embodiment, the peripheral serotonin receptor antagonist is a xylamidine analog having the formula
R¹-A¹-NH—C(═NH)-A²-R², or a pharmaceutically acceptable acid addition salt thereof,
   which is described in UK Patent No. 1,094,985, of which Hodson is an inventor, the contents of which are incorporated by reference, wherein R¹ and R², which may be the same or different, are each a phenyl or thien-2-yl group, optionally substituted in one or more positions by a halogen atom and/or a lower alkyl and/or a lower alkoxy and/or a hydroxy and/or a lower alkylthio and/or a trifluoromethyl and/or a phenyl and/or a phenoxy and/or a phenyl-(lower alkyl) and/or a phenyl-(lower alkoxy) group, each of said phenyl, phenoxy, phenyl-(lower alkyl) and phenyl-(lower alkoxy) groups being optionally substituted in one or more positions by a halogen atom and/or a lower alkyl and/or a lower alkoxy and/or a hydroxy and/or a lower alkylthio group; A¹ is a divalent straight or branched (oxy/thio)-alkylene linkage containing from two to six carbon atoms and optionally one or two divalent oxygen and/or sulfur atom(s), provided that there are at least two carbon atoms between the divalent atom and the —NH— group and between the two divalent atoms; A² is a straight or branched alkylene chain containing from one to four carbon atoms; and wherein in the definitions of R¹ and R², the term lower as applied to alkyl, alkoxy, or alkylthio groups or the alkyl, alkoxy, or alkylthio moieties of a group means an alkyl, alkoxy, or alkylthio group having 1 to 4 carbon atoms, which may be either straight or branched, such as methyl, ethyl, n-propyl, iso-propyl, n-butyl, iso-butyl and tert-butyl groups.

Examples of the xylamidine analogs described in the '985 patent include:
N-(2-3′-methylphenoxypropyl) phenylacetamidine;
N-(2-2′-thenyloxypropyl)-4-chlorophenylacetamidine;
N-(2-3′-ethoxyphenoxypropyl) phenylacetamidine;
N-(2-3′-methoxyphenoxypropyl)-4-bromophenylacetamidine;
N-(2-3′-chlorophenoxyethyl)-4-chlorophenylacetamidine;
N-(2-3′-ethoxyphenoxypropyl)-4-chlorophenylacetamidine;
N-(2-3′-methylphenoxyethyl)-3-methylphenylacetamidine.
N-(2-3′-ethylphenoxypropyl)phenylacetamidine;
N-(2-phenoxypropyl)-4-methylphenylacetamidine;
N-(2-3′-chlorophenoxypropyl)-4-chlorophenylacetamidine;
N-(2-3′-methylbenzyloxypropyl)-4-chlorophenylacetamidine;
N-(2-3′-ethylphenoxypropyl)-4-chlorophenylacetamidine;
N-(2-3′-methoxyphenoxypropyl)-phenylacetamidine;
N-(2-3′-chlorophenoxypropyl)-phenylacetamidine;
N-(2-3′-methoxyphenoxypropyl)-3-chlorophenylacetamidine;
N-(2-3′-methylphenoxypropyl)-4-bromophenylacetamidine;
N-(2-3′-methylphenoxypropyl)-3-chlorophenylacetamidine;
N-(2-3′-propoxyphenoxypropyl)-4-chlorophenylacetamidine;
N-(2-3′-methoxyphenoxypropyl)-3-methoxyphenylacetamidine;
N-(2-3′-methoxyphenoxyethyl)-3-methylphenylacetamidine;
N-(2-3′-methylphenoxypropyl)-4-methylphenylacetamidine;
N-(2-3′-methoxyphenoxypropyl)-4-chlorophenylacetamidine;
N-(1-3′-methylphenoxyprop-2-yl)-4-chlorophenylacetamidine;
N-(2-phenoxybut-3-yl)-4-chlorophenylacetamidine;
N-(2-3′-methylphenoxypropyl-3-methylphenylacetamidine;
N-(1-3′-methoxyphenoxyprop-2-yl)-4-chlorophenylacetamidine;
N-(1-phenoxyprop-2-yl)-3-methylphenylacetamidine;
N-(2-2′-thenyloxypropyl)-3-methylphenylacetamidine;
N-(2-3,5-dimethoxyphenoxypropyl)-3-methylphenylacetamidine;
N-(2-phenoxypropyl)-3,4-dimethylphenylacetamidine;
N-(4-3′-methoxybutyl)-3,4-dimethylphenylacetamidine;
N-(2-2′-thenyloxypropyl)-3,4-dimethylphenylacetamidine;
N-(2-3′-methylphenoxypropyl)-chlorophenylacetamidine;
N-(2-3′-methylphenoxybut-3-yl)-4-chlorophenylacetamidine;
N-(2-3′-methoxyphenoxypropyl)-3,4-dimethylphenylacetamidine;
N-(2-phenoxypropyl)-3-methylphenylacetamidine;
N-(2-3′-methoxyphenoxypropyl)-3-methylphenylacetamidine;
N-(2-3′-methylphenoxypropyl)-3,4-dimethylphenylacetamidine;
N-(1-3′-methoxyphenoxyprop-2-yl)-3,4-dimethylphenylacetamidine;
N-[2-(5-chloro-2-thenyloxy) ethyl]-m-methylphenylacetamidine;
N-[2-(5-chloro-2-thenyloxy) propyl]-3,4-dimethylphenylacetamidine;
N-(2-m-chlorobenzyloxypropyl)-3-methylphenylacetamidine;
N-[2-(5-chloro-2-thenyloxy) propyl]-m-methylphenylacetamidine;
N-(2-m-chlorobenzyloxypropyl)-3,4-dimethylphenylacetamidine;
N-(2-m-methoxyphenoxypropyl)-3,4-dichlorophenylacetamidine;
N-(2-m-fluorophenoxypropyl)-3,4-dimethylphenylacetamidine;
N-(2-m-chlorophenoxypropyl)-3,4-dimethylphenylacetamidine;
N-(2-m-fluorophenoxypropyl)-m-methylphenylacetamidine;
N-(2-o-chlorobenzyloxypropyl)-3,4-dimethylphenylacetamidine;
N-(2-o-chlorobenzyloxypropyl)-m-methylphenylacetamidine;
N-(2-m-bromophenoxypropyl)-m-methylphenylacetamidine;
N-(2-m-bromophenoxypropyl)-3,4-dimethylphenylacetamidine;
N-[2-m-(methylthio) phenoxypropyl]-3,4-dimethylphenylacetamidine;
N-(2-m-methylthiophenoxypropyl)-m-methylphenylacetamidine;
N-(2-m-t-butylphenoxypropyl)-3,4-dimethylphenylacetamidine;
N-(2-phenylthiopropyl)-m-methylphenylacetamidine;
N-(2-m-methoxyphenylthiopropyl)-3,4-dimethylphenylacetamidine;
N-(2-2′-phenoxyethoxyethyl)-3,4-dimethylphenylacetamidine;
N-(2-m-phenoxyphenoxypropyl)-3,4-dimethylphenylacetamidine;
N-(2-m-benzylphenoxypropyl)-3,4-dimethylphenylacetamidine;
N-(2-3′-benzyloxyphenoxypropyl)-3-methylphenylacetamidine;
N-(2-2′-benzyloxyphenoxypropyl)-4-chlorophenylacetamidine;
N-(2-3′-phenylphenoxypropyl)-3,4-dimethylphenylacetamidine;
N-(2-3′-phenylphenoxypropyl)-3-methylphenylacetamidine;
N-2-3′-benzyloxybenzyloxypropyl)-4-chlorophenylacetamidine;
N-(2-4′-benzyloxyphenoxypropyl)-3,4-dimethylphenylacetamidine;
N-(2-3′-methoxyphenoxypropyl)-3-benzyloxyphenylacetamidine; and
N-(2-3′-phenoxyphenoxypropyl)-3-methylphenylacetamidine.

In another embodiment, the peripheral serotonin receptor antagonist is a BW501C67 analog having the formula
R¹⁰-A¹⁰-NH—C(═NH)-A²⁰-NZ-R²⁰, or a pharmaceutically acceptable acid addition salt thereof,
   which is described in UK Patent No. 1,307,978, of which Hodson is an inventor, the contents of which are incorporated by reference, wherein R¹⁰ and R²⁰, which may be the same or different, are each a phenyl or thien-2-yl group, optionally substituted in one or more positions by a halogen atom and/or a lower alkyl and/or a lower alkoxy and/or a hydroxy and/or a lower alkylthio and/or a trifluoromethyl and/or a phenyl and/or a phenoxy and/or a phenyl-(lower alkyl) and/or a phenyl-(lower alkoxy) group, each of said phenyl, phenoxy, phenyl-(lower alkyl) and phenyl-(lower alkoxy) groups being optionally substituted in one or more positions by a halogen atom and/or a lower alkyl and/or a lower alkoxy and/or a hydroxy and/or a lower alkylthio group; A¹⁰ is a divalent straight or branched (oxy/thio)-alkylene linkage containing from two to six carbon atoms and optionally one or two divalent oxygen and/or sulfur atom(s), provided that there are at least two carbon atoms between the divalent atom and the —NH— group and between the two divalent atoms; A²⁰ is a straight or branched alkylene chain containing from one to four carbon atoms; Z is a hydrogen atom or a lower alkyl group; and wherein in the definitions of R¹⁰, R²⁰, and Z, the term lower as applied to alkyl, alkoxy, or alkylthio groups or the alkyl, alkoxy, or alkylthio moieties of a group means an alkyl, alkoxy, or alkylthio group having 1 to 4 carbon atoms, which may be either straight or branched, such as methyl, ethyl, n-propyl, iso-propyl, n-butyl, iso-butyl and tert-butyl groups.

Examples of the BW501C67 analogs described in the '978 patent include N-[2-(3-chlorophenoxy)propyl]-2-(phenylamino)-Ethanimidamide and its hydrochloride salt; N-[2-(3-methoxyphenoxy)propyl]-2-[(3-methylphenyl)amino]-Ethanimidamide and its hydroiodide salt; N-[2-(3-chlorophenoxy)propyl]-2-(phenylamino)-Ethanimidamide and its bydroiodide salt; 2-(phenylamino)-N-[2-[[3-(phenylmethoxy)phenyl]methoxy]propyl]-Ethanimidamide, and its hydriodide salt; 2-[(4-chlorophenyl)amino]-N-[2-(3,4-dimethylphenoxy)propyl]-Ethanimidamide and its hydriodide salt; N-[2-[3-(1,1-dimethylethyl)phenoxy]propyl]-2-(phenylamino)Ethanimidamide and its hydriodide salt; N-[2-[(3-chlorophenyl)methoxy]propyl]-2-(phenylamino)-Ethanimidamide and its hydriodide salt; N-[2-(3-bromophenoxy)propyl]-2-(phenylamino)-Ethanimidamide and its hydriodide salt; N-[2-(3-methoxyphenoxy)propyl]-2-[[3-(trifluoromethyl)phenyl]amino]-Ethanimidamide and its hydriodide salt; 2-[(3-fluorophenyl) amino]-N-(2-phenoxypropyl)-Ethanimidamide and its hydriodide salt; 2-[(3-bromophenyl) amino]-N-(2-phenoxypropyl)-Ethanimidamide and its hydriodide salt; N-(2-phenoxypropyl)-2-(phenylamino)-Ethanimidamide and its hydriodide salt; N-(2-phenoxypropyl)-2-(phenylamino)-Ethanimidamide and its 4-methylbenzenesulfonate salt; N-[2-(3-ethoxyphenoxy)ethyl]-2-(phenylamino)-Ethanimidamide and its 4-methylbenzenesulfonate salt; 2-(phenylamino)-N-[2-(phenylthio)propyl]-Ethanimidamide and its 4-methylbenzenesulfonate salt; N-[2-(3-chlorophenoxy)ethyl]-2-(phenylamino)-Ethanimidamide and its 4-methylbenzenesulfonate salt; 2-[(2,6-dimethylphenyl)amino]-N-(2-phenoxypropyl)Ethanimidamide and its 2-Naphthalenesulfonic acid; N-(2-phenoxyethyl)-2-(phenylamino)Ethanimidamide and its 4-methylbenzenesulfonate salt; 2-(methylphenylamino)-N-(2-phenoxypropyl)Ethanimidamide and its 4-methylbenzenesulfonate salt, N-[2-([1,1′-biphenyl]-3-yloxy)propyl]-2-(phenylamino)Ethanimidamide and its hydriodide salt; N-[2-(3-fluorophenoxy)-N-[2-(2-chlorophenoxy)propyl]-2-[(3,4-dimethylphenyl)amino]Ethanimidamide and its hydriodide salt; N-[2-[(5-chloro-2-thienyl)methoxy]propyl]-2-[(3,4-dimethylphenyl)amino]Ethanimidamide and its hydriodide salt; 2-[(4-chlorophenyl) amino]-N-[2-[(3-methylphenyl)methoxy]propyl]Ethanimidamide and its hydriodide salt; 2-1 (4-chlorophenyl)amino]-N-[2-(3-methylphenoxy)propyl]Ethanimidamide and its hydriodide salt; 2-[(3,4-dimethylphenyl)amino]-N-[2-(3-methoxyphenoxy)propyl]Ethanimidamide and its hydriodide salt; 2-(phenylamino)-N-[2-(phenylmethoxy)propyl]Ethanimidamide and its hydriodide salt; 2-[(4-chlorophenyl)amino]-N-(2-phenoxypropyl)Ethanimidamide and its hydriodide salt; 2-[(3-chlorophenyl)amino]-N-[2-(3-methoxyphenoxy)propyl]Ethanimidamide and its hydroiodide salt; and 2-[(3-chlorophenyl)amino]-N-[2-(3-methoxyphenoxy)propyl]-2-[(3-chlorophenyl)Ethanimidamide and its hydroiodide salt.

The activity of the compounds and the acid addition salts thereof resides in the bases. Therefore, the acid in the acid addition salts is of minor importance though it is preferably pharmacologically and pharmaceutically acceptable. The acid addition salts are derived from non-toxic acids. The acids employed will normally be those currently recognized to be most desirable from both pharmacological and pharmaceutical standpoints, which shall herein be designated as “pharmaceutically acceptable” acids. Instances of suitable acid addition salts formed with pharmaceutically acceptable acid anions include those formed with hydrochloric acid, hydrobromic acid, hydriodic acid, sulphuric acid, para-toluene-sulphonic acid, maleic acid, lactic acid, citric acid, tartaric acid, succinic acid, oxalic acid, para-chloro-benzene-sulphonic acid, and many others.

In an embodiment, xylamidine and/or its analogs are administered at a dose from about 5 to about 200 mg. and in another embodiment, from about 10 to about 100 mg, especially if given orally. For example, xylamidine and/or its analogs may be administered to a patient orally in a dose of about 10 mg, or about 20 mg, or about 25 mg, or about 30 mg, or about 40 mg, or about 50 mg, or about 60mg, or about 70 mg, or about 75 mg, or about 80 mg, or about 90 mg or about 100 mg. In another embodiment, xylamidine and/or its analogs are given parenterally at a dose from about 0.5 mg to about 20 mg and in another embodiment, from about 1 mg to about 10 mg. For example, xylamidine and/or its analogs may be administered to a patient parenterally in a dose of about 0.5 mg or about 1mg, or about 2 mg, or about 3 mg, or about 4 mg, or about 5 mg, or about 6 mg, or about 7 mg, or about 8 mg, or about 9 mg, or about 10 mg, or about 12 mg, or about 15 mg, or about 20 mg.

In an embodiment, the peripheral serotonin receptor antagonist is BW501C67, having the structure below:

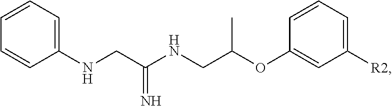

wherein R2 is Cl, or a pharmaceutically acceptable acid addition thereof, However, the present disclosure contemplates analogs of BW501C67, wherein R2is Br, F, H, OMe, benzyl, or alkyl containing 1-4 carbon atoms, such as methyl, ethyl, n-propyl, isopropyl, n-butyl, sec-butyl, or t-butyl, or a pharmaceutically acceptable acid addition thereof,

In another embodiment, the present disclosure contemplates another analog of BW501C67:

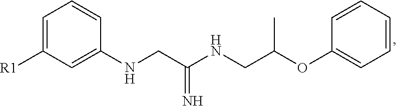

or a pharmaceutically acceptable acid addition salt thereof, wherein R1 is H, F, Cl, Br, Me, or OMe.

In another embodiment, the present disclosure contemplates another analog of BW501C67:

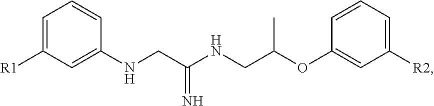

or a pharmaceutically acceptable acid addition salt thereof, wherein R1 is F, Cl, Br, Me, or OMe and R2 is Br, Cl, F, OMe, benzyl, or alkyl containing 1-4 carbon atoms.

BW501C67 and/or analogs thereof are administered at a dose from about 1 mg to about 100 mg and in another embodiment, from about 2 mg to about 20 mg, especially if given orally. For example, BW501C67 or analogs thereof may be administered to a patient orally in a dose of about 1 mg or about 2 mg, or about 3 mg, or about 4 mg, or about 5 mg, or about 6 mg, or about 7 mg, or about 8 mg, or about 9 mg, or about 10 mg, or about 20 mg, or about 25 mg, or about 30 mg, or about 40 mg, or about 50 mg, or about 60 mg, or about 70 mg, or about 75 mg, or about 80 mg, or about 90 mg, or about 100 mg. In another embodiment, BW501C67 or analogs thereof are given parenterally at a dose from about 0.1 mg to about 10 mg and in another embodiment, from about 0.2 mg to about 2 mg. For example, BW501C67 or analogs thereof may be administered to a patient parenterally in a dose of about 0.1 mg, or about 0.2 mg, or about 0.3 mg, or about 0.4 mg, or about 0.5 mg, or about 0.6 mg, or about 0.7 mg, or about 0.8 mg, or about 0.9 mg. or about 1 mg, or about 2 mg, or about 3 mg. or about 4 mg, or about 5 mg, or about 6 mg, or about 7 mg, or about 8 mg, or about 9 mg. or about 10 mg, or about 12 mg or about 15 mg, or about 20 mg.

In some embodiments, the above doses correspond to the total dose administered in once instance to an adult human. In some embodiments, said adult human weighs between about 40 kg and about 150 kg, for example, about 70 kg.

In some embodiments, the selected dose of the peripheral serotonin receptor antagonist is the minimal dose required to provide the desired degree of attenuation of the peripheral side effects of the 5-HT2A receptor antagonist.

### 5-HT2A Receptor Agonists

In some embodiments, a 5-HT2A receptor in the central nervous system is activated selectively. In some embodiments, one or more additional serotonin receptor in the central nervous system selected from the group consisting of 5-HT1A, 5-HT2B, and 5-HT2C is activated.

In some embodiments, a 5-HT2A receptor agonist is selected from a group consisting of an ergoline, a tryptamine, a phenethylamine, and an amphetamine.

In some embodiments, a 5-HT2A receptor agonist is a tryptamine. In some embodiments, a tryptamine is selected from the group consisting of psilocybin, psilocin, N,N-dimethyltryptamine (DMT), 5-methoxy-N,N-dimethyltryptamine (5-MeO-DMT), N-methyl-N-ethyltryptamine (MET), N-methyl-N-isopropyltryptamine (MIPT), N,N-diethyltryptamine (DET), N,N-diisopropyltryptamine (DIPT), N,N-dipropyltryptamine (DPT), N-ethyl-N-propyltryptamine (EPT), 5-methoxy-N-methyl-N-isopropyltryptamine (5-MeO-MIPT), 5-methoxy-N,N-diisopropyltryptamine (5-MeO-DIPT), 5-methoxy-N-methyl-N-ethyltryptamine (5-MeO-MET), 5-methoxy-N,N-diethyltryptamine (5-MeO-DET), N,N-diallyl-5-methoxytryptamine (5-MeO-DALT), 4-hydroxy-N-methyl-N-ethyltryptamine (4-HO-MET), 4-hydroxy-N-methyl-N-isopropyltryptamine (4-HO-MIPT), 4-hydroxy-N,N-diisopropyltryptamine (4-HO-DIPT), 4-hydroxy-N,N-diethyltryptamine (4-HO-DET), 4-hydroxy-N,N-dipropyltryptamine (4-HO-DPT), 4-hydroxy-N-ethyl-N-propyltryptamine (4-HO-EPT), 4-acetoxy-N-methyl-N-ethyltryptamine (4-AcO-MET), 4-acetoxy-N-methyl-N-isopropyltryptamine (4-AcO-MIPT), 4-acetoxy-N,N-diisopropyltryptamine (4-AcO-DIPT), 4-acetoxy-N,N-diethyltryptamine (4-AcO-DET), 4-acetoxy-N,N-dipropyltryptamine (4-AcO-DPT), 4-acetoxy-N-ethyl-N-propyltryptamine (4-AcO-EPT), 4-acetoxy-N,N-dimethyltryptamine (4-AcO-DMT), alpha-methyltryptamine (AMT), alpha-ethyltryptamine (AET), and 5-methoxy-alpha-methyltryptamine (5-MeO-AMT).

In some embodiments, a 5-HT2A receptor agonist is an ergoline. In some embodiments, an ergoline is a lysergic acid amide. In some embodiments, a lysergic acid amide is selected from a group consisting of lysergic acid diethylamide (LSD), lysergic acid 2,4-dimethylazetidide (LSZ), 6-ethyl-6-nor-lysergic acid diethylamide (ETH-LAD), 6-propyl-6-nor-lysergic acid diethylamide (PRO-LAD), 1-acetyl-lysergic acid diethylamide (ALD-52), 1-propionyl-lysergic acid diethylamide (1P-LSD), 1-butyryl-lysergic acid diethylamide (1B-LSD), and 1-(cyclopropylmethanoyl)-lysergic acid diethylamide (1cP-LSD).

In some embodiments, a 5-HT2A receptor agonist is a phenethylamine. In some embodiments, a phenethylamine is selected from the group consisting of mescaline, escaline, proscaline, methallylescaline, allylescaline, 4-bromo-2,5-dimethoxypenethylamine (2C-B), 4-chloro-2,5-dimethoxypenethylamine (2C-C), 4-iodo-2,5-dimethoxypenethylamine (2C-I), 2,5-dimethoxy-4-methylphenethylamine (2C-D), 2-(4-Ethyl-2,5-dimethoxyphenyl)ethanamine (2C-E), 2-(2,5-Dimethoxy-4-propylphenyl)ethan-1-amine (2C-P), 2-[4-(Ethylsulfanyl)-2,5-dimethoxyphenyl]ethan-1-amine (2C-T-2), 2-[2,5-Dimethoxy-4-(propylsulfanyl)phenyl]ethan-1-amine (2C-T-7), 2-(4-iodo-2,5-dimethoxyphenyl)-N-[(2-methoxyphenyl)methyl]ethanamine (25I-NBOMe), 2-(4-bromo-2,5-dimethoxyphenyl)-N-[(2-methoxyphenyl)methyl]ethanamine (25B-NBOMe), 2-(4-chloro-2,5-dimethoxyphenyl)-N-[(2-methoxyphenyl)methyl]ethanamine (25C-NBOMe), 2-(4-methyl-2,5-dimethoxyphenyl)-N-[(2-methoxyphenyl)methyl]ethanamine (25D-NBOMe), 2-(4-ethyl-2,5-dimethoxyphenyl)-N-[(2-methoxyphenyl)methyl]ethanamine (25E-NBOMe), 2-(4-iodo-2,5-dimethoxyphenyl)-N-[(2-hydroxyphenyl)methyl]ethanamine (25I-NBOH), 2-(4-bromo-2,5-dimethoxyphenyl)-N-[(2-hydroxyphenyl)methyl]ethanamine (25B-NBOH), 2-(4-chloro-2,5-dimethoxyphenyl)-N-[(2-hydroxyphenyl)methyl]ethanamine (25C-NBOH), 2-(4-methyl-2,5-dimethoxyphenyl)-N-[(2-hydroxyphenyl)methyl]ethanamine (25D-NBOH), 2-(4-ethyl-2,5-dimethoxyphenyl)-N-[(2-hydroxyphenyl)methyl]ethanamine (25E-NBOH), and 2-(4-cyano-2,5-dimethoxyphenyl)-N-[(2-hydroxyphenyl)methyl]ethanamine (25CN-NBOH).

In some embodiments, a 5-HT2A receptor agonist is an amphetamine. In some embodiments, an amphetamine is selected from the group consisting of 2,5-dimethoxy-4-methylamphetamine (DOM), 2,5-dimethoxy-4-bromoamphetamine (DOB), 2,5-dimethoxy-4-chloroamphetamine (DOC.) 2,5-dimethoxy-4-iodoamphetamine (DOI), 2,5-dimethoxy-4-ethylamphetamine (DOET), and 2,5-Dimethoxy-4-propylamphetamine (DOPR).

In some embodiments, a 5-HT2A receptor agonist is selected from the group consisting of psilocybin, 4-AcO-DMT, psilocin, DMT, 5-MeO-DMT, LSD, mescaline, 2C-B, 2C-E, 2C-T-2, 2C-T-7, and DOM.

In some embodiments, a 5-HT2A receptor agonist is a compound according to Formula I:

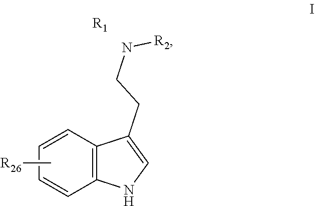

or a pharmaceutically acceptable salt thereof,

wherein

R₁ is optionally substituted C₁-C₄ aliphatic;

R₂ is optionally substituted C₁-C₄ aliphatic;

R₂₆ is selected from the group consisting of hydrogen, halogen, —CN, —OH, C₁-C₃ alkoxy, C₁-C₃ haloalkyl, OAc, —OPO(OH)₂ and NH₂.

In some embodiments, R₁ is selected from the group consisting of Me, Et, nPr, iPr, cyclopropyl, allyl, isobutyl, cyclopropylmethyl. In some embodiments, R₂ is selected from the group consisting of Me, Et, nPr, iPr, cyclopropyl, allyl, isobutyl, cyclopropylmethyl.

In some embodiments, R₂₆ is selected from the group consisting of hydrogen, F, Cl, Br, I, CF₃, Me, CN, OMe, OH, OAc, and NH₂. In some embodiments, R₂₆ is selected from the group consisting of F, Cl, Br, I, CF₃, Me, CN, OMe, OH, OAc, and NH₂. In some embodiments, R₂₆ is halogen. In some embodiments, R₂₆ is fluoro. In some embodiments, R₂₆ is chloro. In some embodiments, R₂₆ is bromo. In some embodiments, R₂₆ is iodo.

In some embodiments, a 5-HT2A receptor agonist is a compound selected from the group consisting of:

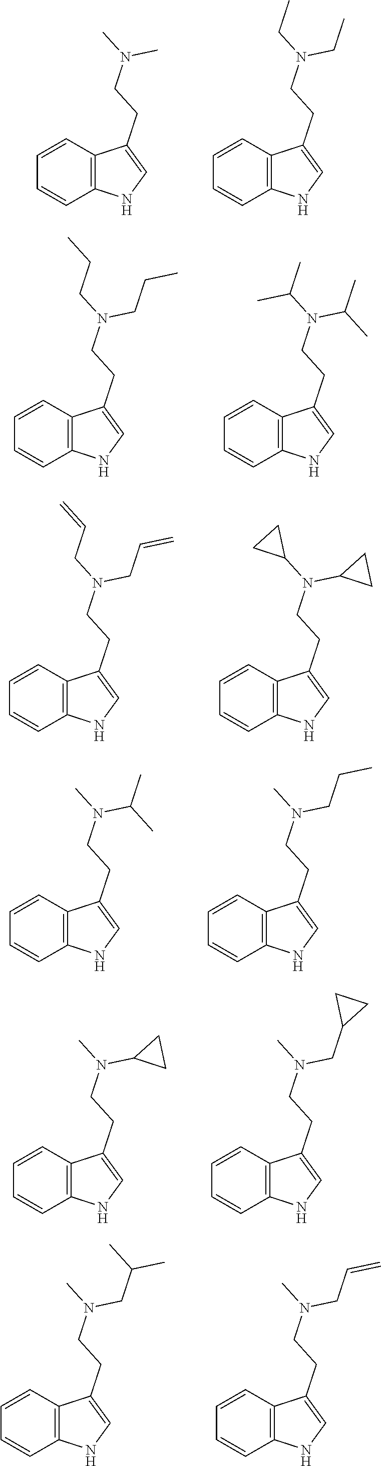

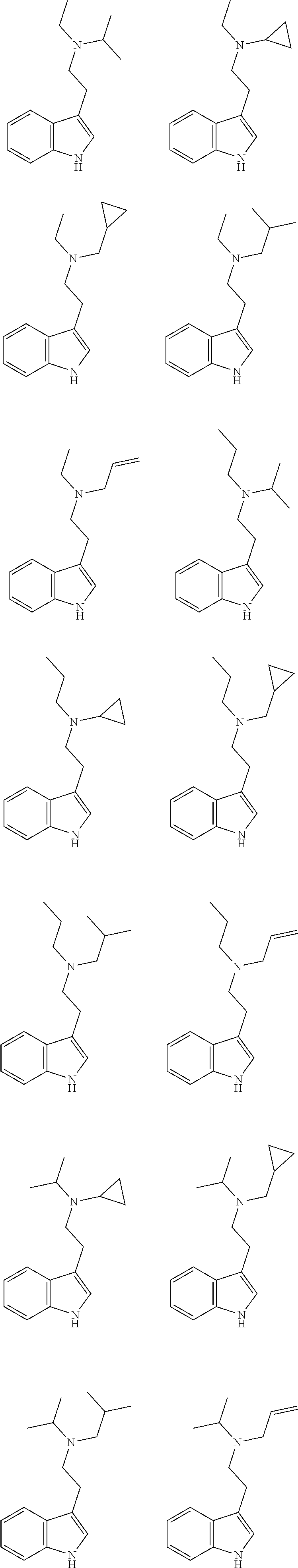

or a pharmaceutically acceptable salt thereof.

In some embodiments, a 5-HT2A receptor agonist is a compound selected from the group consisting of:

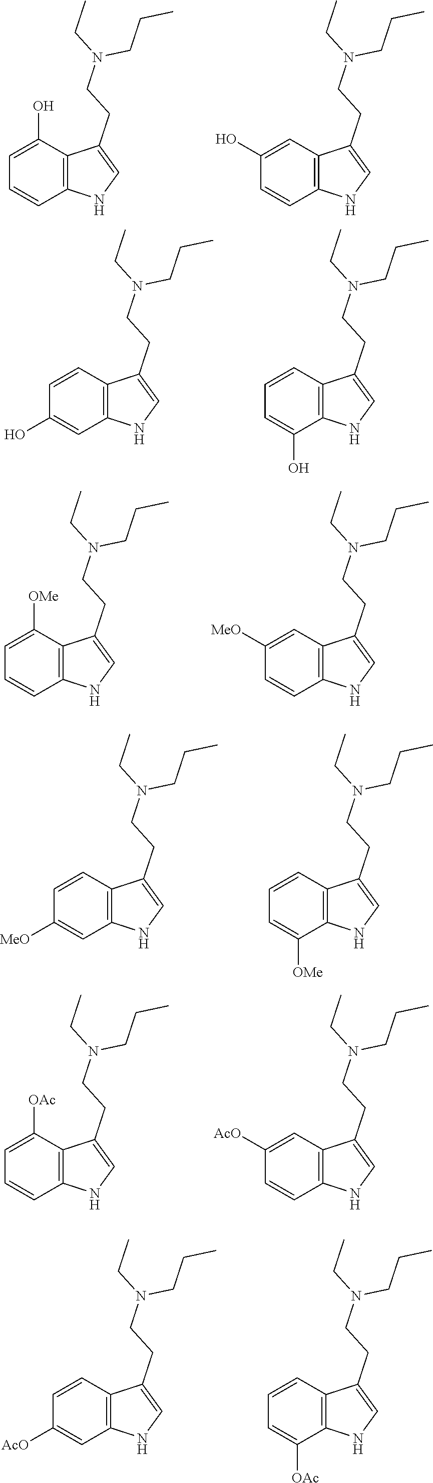

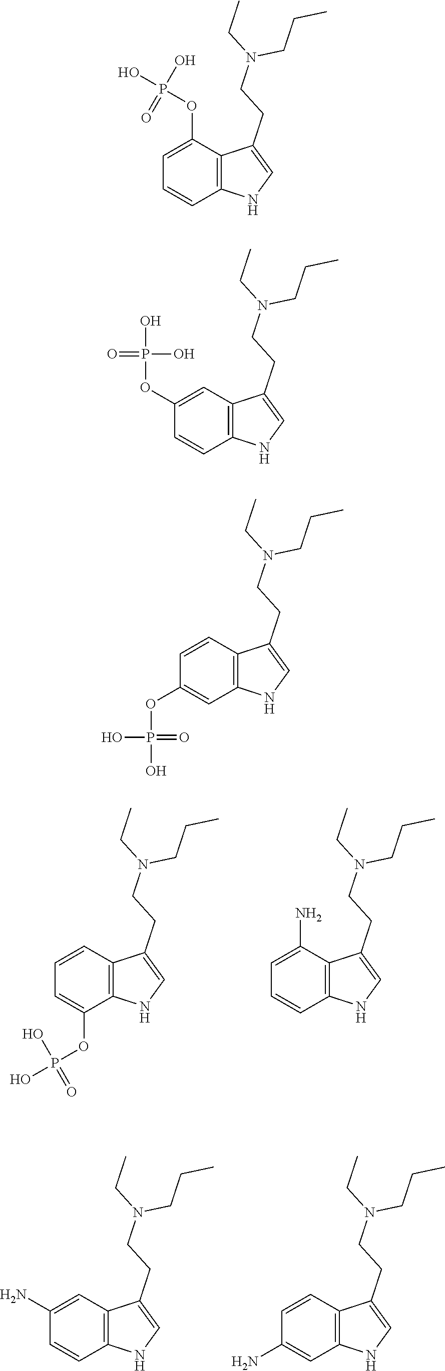

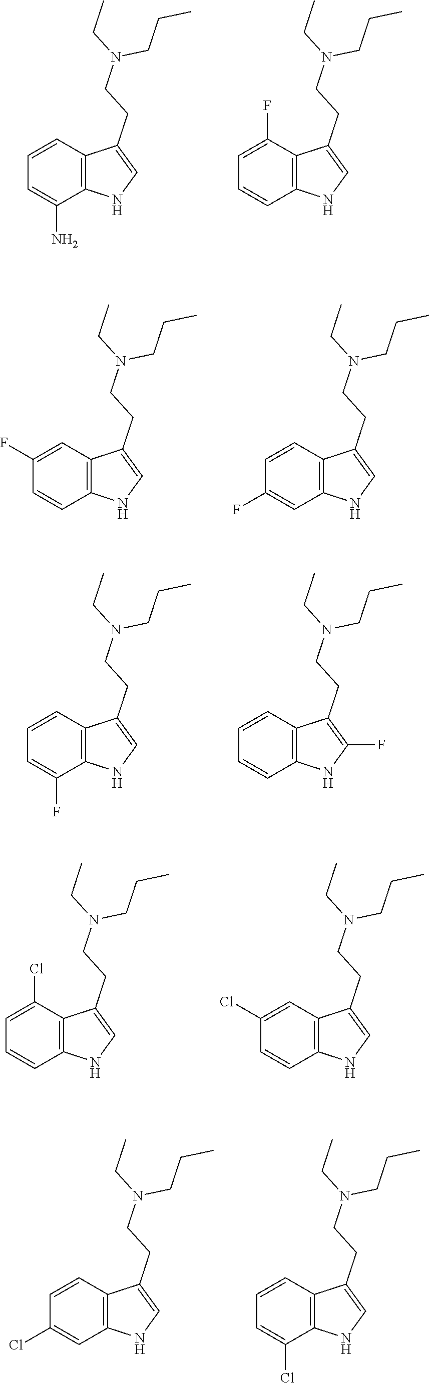

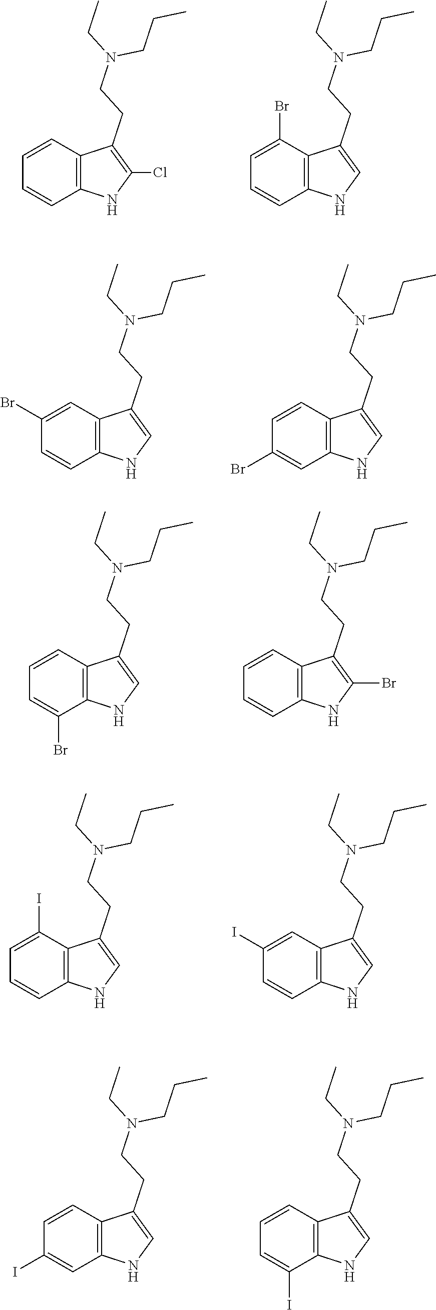

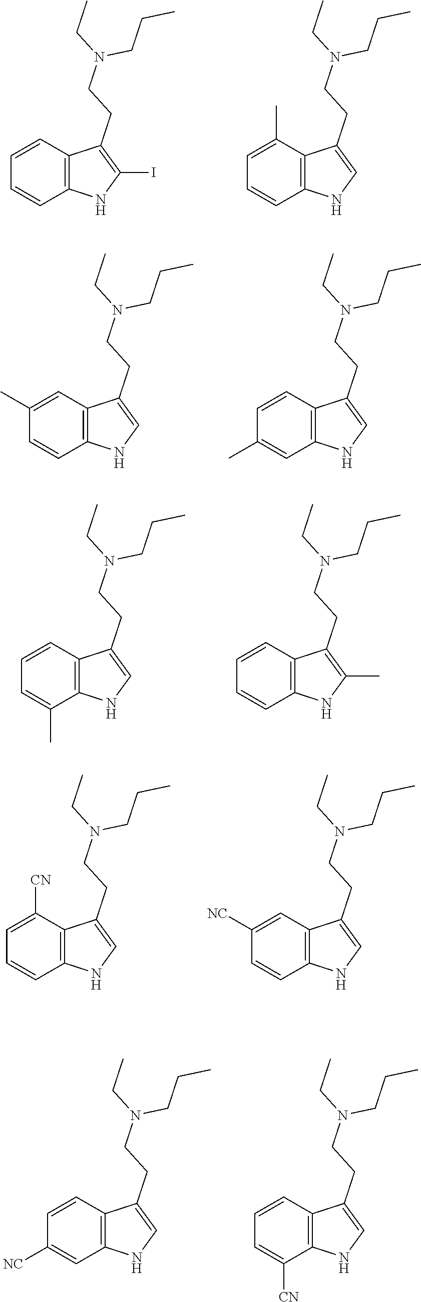

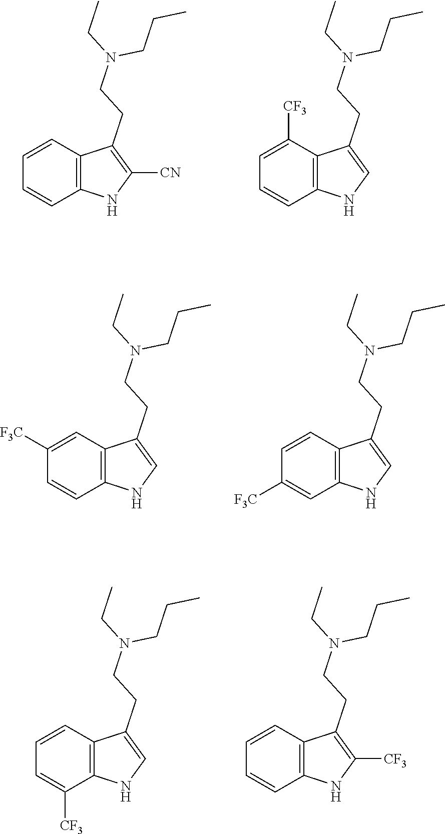 [SMILES: CCCN(CC)CCc1c(C#N)[nH]c2ccccc12 ; CCCN(CC)CCc1c[nH]c2cccc(C(F)(F)F)c12 ; CCCN(CC)CCc1c[nH]c2ccc(C(F)(F)F)cc12 ; CCCN(CC)CCc1c[nH]c2cc(C(F)(F)F)ccc12 ; CCCN(CC)CCc1c[nH]c2c(C(F)(F)F)cccc12 ; CCCN(CC)CCc1c(C(F)(F)F)[nH]c2ccccc12]

or a pharmaceutically acceptable salt thereof.

In some embodiments, a 5-HT2A receptor agonist is a compound selected from the group consisting of:

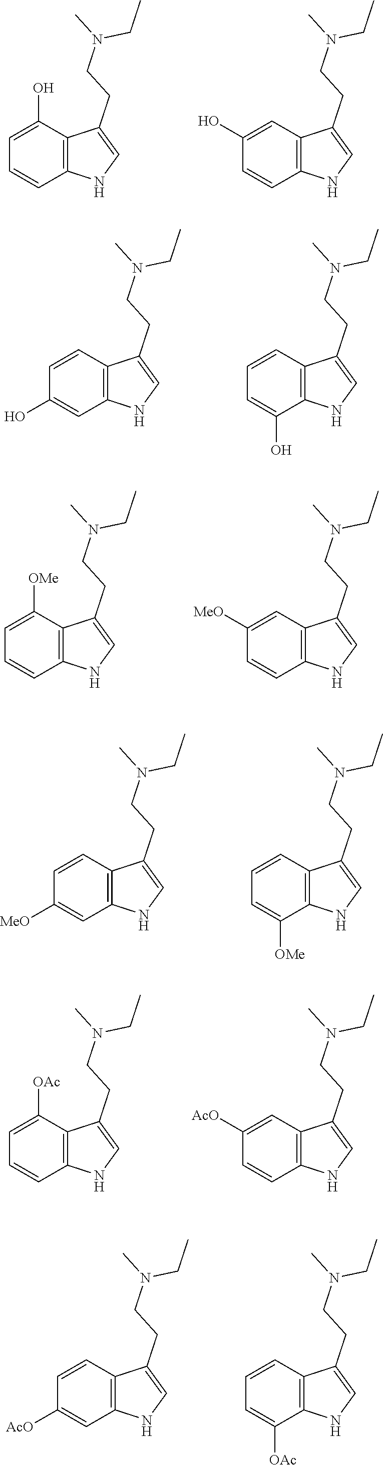

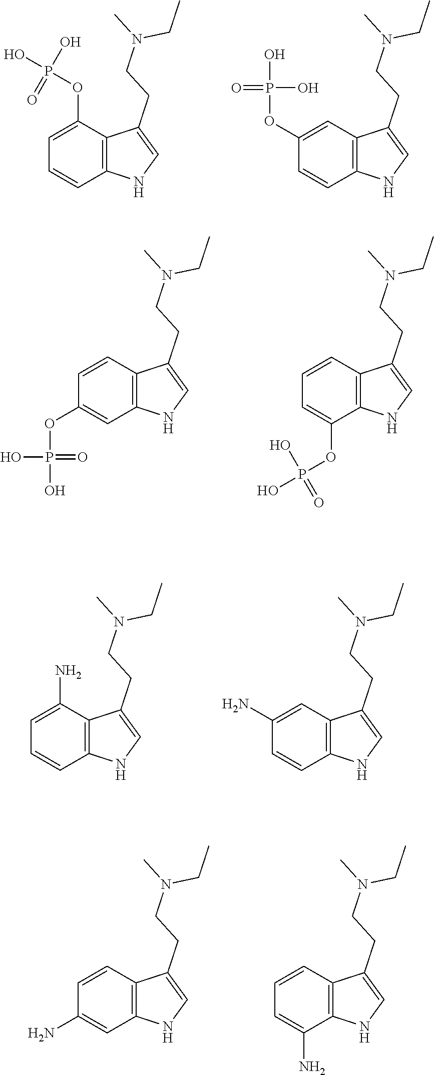

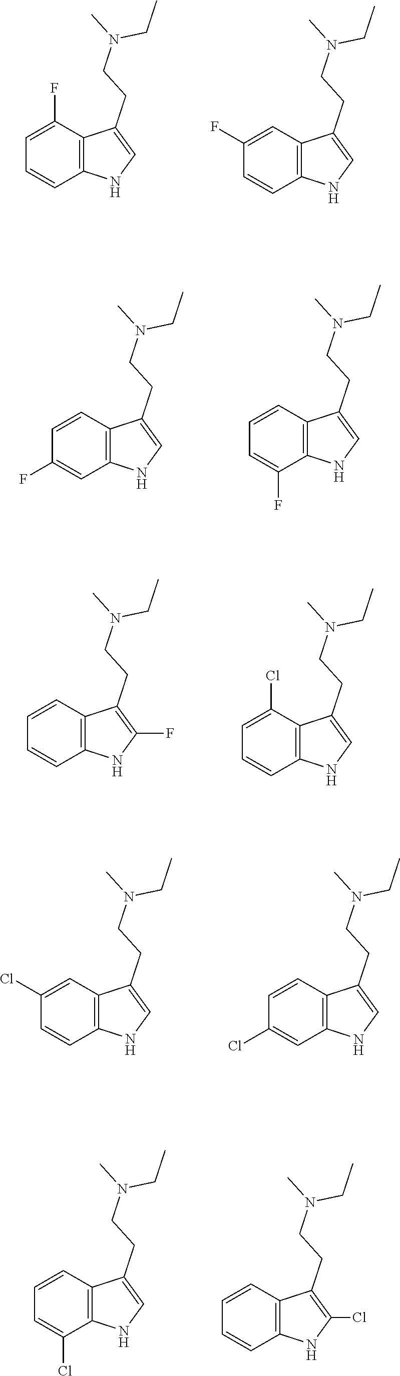

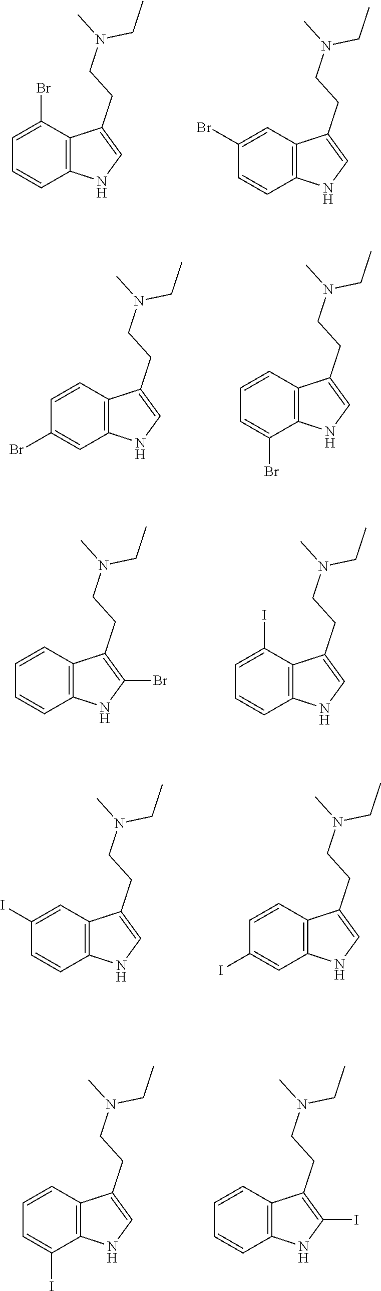

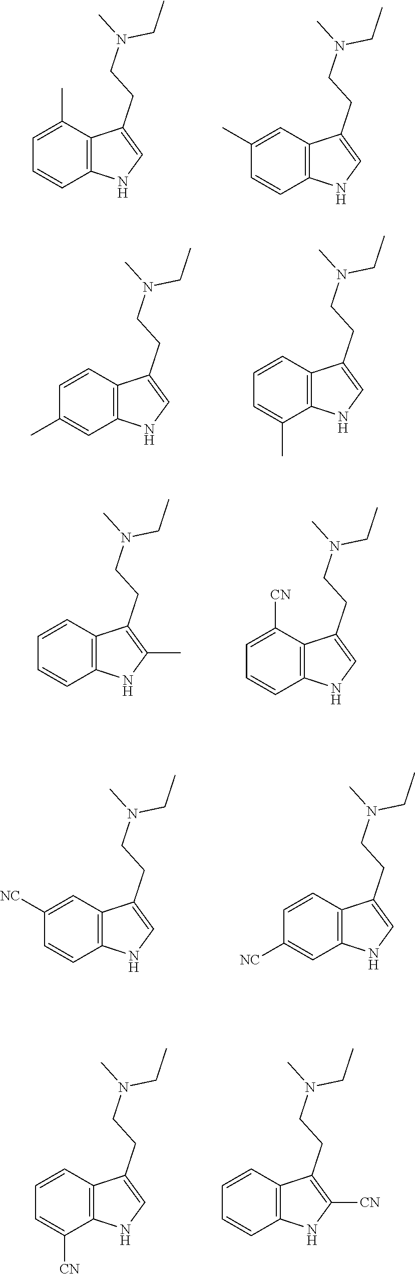

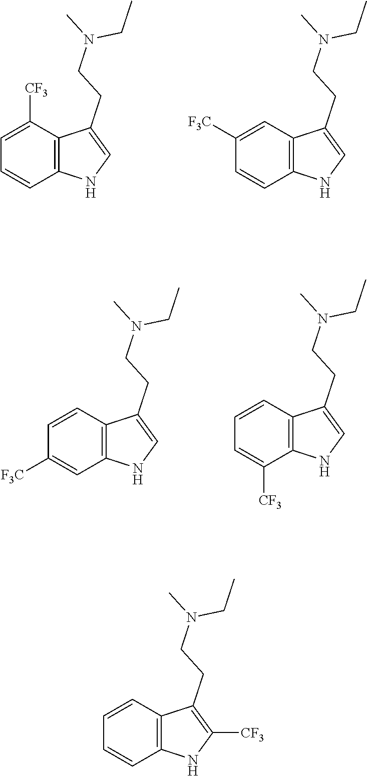 [SMILES: CCN(C)CCc1c[nH]c2cccc(C(F)(F)F)c12 ; CCN(C)CCc1c[nH]c2ccc(C(F)(F)F)cc12 ; CCN(C)CCc1c[nH]c2cc(C(F)(F)F)ccc12 ; CCN(C)CCc1c[nH]c2c(C(F)(F)F)cccc12 ; CCN(C)CCc1c(C(F)(F)F)[nH]c2ccccc12]

or a pharmaceutically acceptable salt thereof.

In some embodiments, a 5-HT2A receptor agonist is represented by

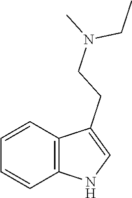

or a pharmaceutically acceptable salt thereof.

In some embodiments, a 5-HT2A receptor agonist is represented by

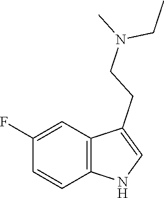 [SMILES: CCN(C)CCc1c[nH]c2ccc(F)cc12]

or a pharmaceutically acceptable salt thereof.

The 5-HT2A receptor agonists are administered to patients in doses in therapeutically effective amounts to treat the psychiatric disorders, as defined herein. In some embodiments, the dose of the 5-HT2A receptor agonist is sufficient to induce hallucinogenic effects in the patient. In some embodiments, a 5-HT2A receptor agonist is selected from the group consisting of lysergic acid diethylamide (LSD), lysergic acid 2,4-dimethylazetidide (LSZ), 6-ethyl-6-nor-lysergic acid diethylamide (ETH-LAD), 6-propyl-6-nor-lysergic acid diethylamide (PRO-LAD), 1-acetyl-lysergic acid diethylamide (ALD-52), 1-propionyl-lysergic acid diethylamide (1P-LSD), 1-butyryl-lysergic acid diethylamide (1B-LSD), and 1-(cyclopropylmethanoyl)-lysergic acid diethylamide (1cP-LSD). In some embodiments, a 5-HT2A receptor agonist is administered at a dose between about 0.010 mg and about 1.0 mg. In some embodiments, a 5-HT2A receptor agonist is administered at a dose selected from the group consisting of about 0.010 mg, about 0.025 mg, about 0.050 mg, about 0.075mg, about 0.10 mg, about 0.20 mg, about 0.30 mg, about 0.40 mg, about 0.50 mg, about 0.60 mg, about 0.70 mg, about 0.80 mg, about 0.90 mg, and about 1.0 mg. In some embodiments, a 5-HT2A receptor agonist is administered at a dose of about 0.010 mg. In some embodiments, a 5-HT2A receptor agonist is administered at a dose of about 0.025 mg. In some embodiments, a 5-HT2A receptor agonist is administered at a dose of about 0.050 mg. In some embodiments, a 5-HT2A receptor agonist is administered at a dose of about 0.075 mg. In some embodiments, a 5-HT2A receptor agonist is administered at a dose of about 0.10 mg. In some embodiments, a 5-HT2A receptor agonist is administered at a dose of about 0.20 mg. In some embodiments, a 5-HT2A receptor agonist is administered at a dose of about 0.30 mg. In some embodiments, a 5-HT2A receptor agonist is administered at a dose of about 0.40 mg. In some embodiments, a 5-HT2A receptor agonist is administered at a dose of about 0.50 mg. In some embodiments, a 5-HT2A receptor agonist is administered at a dose of about 0.60 mg. In some embodiments, a 5-HT2A receptor agonist is administered at a dose of about 0.70 mg. In some embodiments, a 5-HT2A receptor agonist is administered at a dose of about 0.80 mg. In some embodiments, a 5-HT2A receptor agonist is administered at a dose of about 0.90 mg. In some embodiments, a 5-HT2A receptor agonist is administered at a dose of about 1.0 mg.

In some embodiments, a 5-HT2A receptor agonist is selected from the group consisting of 2,5-dimethoxy-4-methylamphetamine (DOM), 2,5-dimethoxy-4-bromoamphetamine (DOB), 2,5-dimethoxy-4-chloroamphetamine (DOET) 2,5-dimethoxy-4-iodoamphetamine (DOI), 2,5-dimethoxy-4-ethylamphetamine (DOET), 2,5-Dimethoxy-4-propylamphetamine (DOPR), and 5-MeO-AMT. In some embodiments, a 5-HT2A receptor agonist is administered at a dose between about 0.10 mg and about 10 mg. In some embodiments, a 5-HT2A receptor agonist is administered at a dose selected from the group consisting of about 0.10 mg, about 0.25 mg, about 0.50 mg, about 0.75 mg, about 1.0mg, about 1.5 mg, about 2.0 mg, about 3.0 mg, about 4.0 mg, about 5.0 mg, about 6.0 mg, about 7.0mg, about 8.0 mg, about 9.0 mg, and about 10 mg. In some embodiments, a 5-HT2A receptor agonist is administered at a dose of about 0.10 mg. In some embodiments, a 5-HT2A receptor agonist is administered at a dose of about 0.25 mg. In some embodiments, a 5-HT2A receptor agonist is administered at a dose of about 0.75 mg. In some embodiments, a 5-HT2A receptor agonist is administered at a dose of about 1.0 mg. In some embodiments, a 5-HT2A receptor agonist is administered at a dose of about 2.0 mg. In some embodiments, a 5-HT2A receptor agonist is administered at a dose of about 3.0 mg. In some embodiments, a 5-HT2A receptor agonist is administered at a dose of about 4.0 mg. In some embodiments, a 5-HT2A receptor agonist is administered at a dose of about 5.0 mg. In some embodiments, a 5-HT2A receptor agonist is administered at a dose of about 6.0 mg. In some embodiments, a 5-HT2A receptor agonist is administered at a dose of about 7.0 mg. In some embodiments, a 5-HT2A receptor agonist is administered at a dose of about 8.0 mg. In some embodiments, a 5-HT2A receptor agonist is administered at a dose of about 9.0 mg. In some embodiments, a 5-HT2A receptor agonist is administered at a dose of about 10.0 mg.

In some embodiments, a 5-HT2A receptor agonist is selected from the group consisting of psilocybin, psilocin, 5-MeO-MIPT, 5-MeO-DIPT, 5-MeO-MET, 5-McO-DET, 5-MeO-DALT, 4-HO-MET, 4-HO-MIPT, 4-HO-DIPT, 4-HO-DET, 4-HO-EPT, 4-ACO-MET, 4-ACO-MIPT, 4-AcO-DIPT, 4-ACO-DET, 4-ACO-EPT, 4-AcO-DMT, alpha-methyltryptamine, 2C-B, 2C-C, 2C-I, 2C-E, 2C-P, 2C-T-2, and 2C-T-7. In some embodiments a 5-HT2A receptor agonist is administered at a dose between about 1.0 mg and about 50 mg. In some embodiments, a 5-HT2A receptor agonist is administered at a dose selected from the group consisting of about 1.0 mg, about 2.5 mg, about 5.0 mg, about 7.5 mg, about 10 mg. about 15 mg, about 20 mg, about 25 mg, about 30 mg, about 35 mg, about 40 mg, about 45 mg, and about 50 mg. In some embodiments, a 5-HT2A receptor agonist is administered at a dose of about 1.0 mg. In some embodiments, a 5-HT2A receptor agonist is administered at a dose of about 2.5 mg. In some embodiments, a 5-HT2A receptor agonist is administered at a dose of about 5.0 mg. In some embodiments, a 5-HT2A receptor agonist is administered at a dose of about 7.5 mg. In some embodiments, a 5-HT2A receptor agonist is administered at a dose of about 10 mg. In some embodiments, a 5-HT2A receptor agonist is administered at a dose of about 15 mg. In some embodiments, a 5-HT2A receptor agonist is administered at a dose of about 20 mg. In some embodiments, a 5-HT2A receptor agonist is administered at a dose of about 25 mg. In some embodiments, a 5-HT2A receptor agonist is administered at a dose of about 30 mg. In some embodiments, a 5-HT2A receptor agonist is administered at a dose of about 35 mg. In some embodiments, a 5-HT2A receptor agonist is administered at a dose of about 40 mg. In some embodiments, a 5-HT2A receptor agonist is administered at a dose of about 45 mg. In some embodiments, a 5-HT2A receptor agonist is administered at a dose of about 50 mg.

In some embodiments, a 5-HT2A receptor agonist is selected from the group consisting of MIPT, 2C-D, escaline, proscaline, methallylescaline, and allylescaline. In some embodiments, a 5-HT2A agonist is administered at a dose between about 2.0 mg and about 100 mg. In some embodiments, a 5-HT2A receptor agonist is administered at a dose selected from the group consisting of about 2.0 mg, about 5.0 mg, about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, and about 100 mg. In some embodiments, a 5-HT2A receptor agonist is administered at a dose of about 2.0 mg. In some embodiments, a 5-HT2A receptor agonist is administered at a dose of about 5.0 mg. In some embodiments, a 5-HT2A receptor agonist is administered at a dose of about 10 mg. In some embodiments, a 5-HT2A receptor agonist is administered at a dose of about 15 mg. In some embodiments, a 5-HT2A receptor agonist is administered at a dose of about 20 mg. In some embodiments, a 5-HT2A receptor agonist is administered at a dose of about 25 mg. In some embodiments, a 5-HT2A receptor agonist is administered at a dose of about 30 mg. In some embodiments, a 5-HT2A receptor agonist is administered at a dose of about 35 mg. In some embodiments, a 5-HT2A receptor agonist is administered at a dose of about 40 mg. In some embodiments, a 5-HT2A receptor agonist is administered at a dose of about 45 mg. In some embodiments, a 5-HT2A receptor agonist is administered at a dose of about 50 mg. In some embodiments, a 5-HT2A receptor agonist is administered at a dose of about 60 mg. In some embodiments, a 5-HT2A receptor agonist is administered at a dose of about 70 mg. In some embodiments, a 5-HT2A receptor agonist is administered at a dose of about 80 mg. In some embodiments, a 5-HT2A receptor agonist is administered at a dose of about 90 mg. In some embodiments, a 5-HT2A receptor agonist is administered at a dose of about 100 mg.

In some embodiments, a 5-HT2A receptor agonist is selected from the group consisting of DET, DIPT, 4-HO-DPT, and 4-AcO-DPT. In some embodiments, a 5-HT2A receptor agonist is administered at a dose between about 5.0 and about 150 mg. In some embodiments, a 5-HT2A receptor agonist is administered at a dose selected from the group consisting of about 5.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, and about 150 mg. In some embodiments, a 5-HT2A receptor agonist is administered at a dose of about 5.0 mg. In some embodiments, a 5-HT2A receptor agonist is administered at a dose of about 10 mg. In some embodiments, a 5-HT2A receptor agonist is administered at a dose of about 20 mg. In some embodiments, a 5-HT2A receptor agonist is administered at a dose of about 30 mg. In some embodiments, a 5-HT2A receptor agonist is administered at a dose of about 40 mg. In some embodiments, a 5-HT2A receptor agonist is administered at a dose of about 50 mg. In some embodiments, a 5-HT2A receptor agonist is administered at a dose of about 60 mg. In some embodiments, a 5-HT2A receptor agonist is administered at a dose of about 70 mg. In some embodiments, a 5-HT2A receptor agonist is administered at a dose of about 80 mg. In some embodiments, a 5-HT2A receptor agonist is administered at a dose of about 90 mg. In some embodiments, a 5-HT2A receptor agonist is administered at a dose of about 100 mg. In some embodiments, a 5-HT2A receptor agonist is administered at a dose of about 110 mg. In some embodiments, a 5-HT2A receptor agonist is administered at a dose of about 120 mg. In some embodiments, a 5-HT2A receptor agonist is administered at a dose of about 130 mg. In some embodiments, a 5-HT2A receptor agonist is administered at a dose of about 140 mg. In some embodiments, a 5-HT2A receptor agonist is administered at a dose of about 150 mg.

In some embodiments, a 5-HT2A receptor agonist is EPT or DPT. In some embodiments, a 5-HT2A receptor is administered at a dose between about 20 and about 400 mg. In some embodiments, a 5-HT2A receptor agonist is administered at a dose selected from the group consisting of about 20 mg, about 40 mg, about 60 mg, about 80 mg, about 100 mg, about 150 mg, about 200 mg, about 250 mg, about 300 mg, and about 400 mg. In some embodiments, a 5-HT2A receptor agonist is administered at a dose of about 20 mg. In some embodiments, a 5-HT2A receptor agonist is administered at a dose of about 40 mg. In some embodiments, a 5-HT2A receptor agonist is administered at a dose of about 60 mg. In some embodiments, a 5-HT2A receptor agonist is administered at a dose of about 80 mg. In some embodiments, a 5-HT2A receptor agonist is administered at a dose of about 100 mg. In some embodiments, a 5-HT2A receptor agonist is administered at a dose of about 150 mg. In some embodiments, a 5-HT2A receptor agonist is administered at a dose of about 200 mg. In some embodiments, a 5-HT2A receptor agonist is administered at a dose of about 250 mg. In some embodiments, a 5-HT2A receptor agonist is administered at a dose of about 300 mg. In some embodiments, a 5-HT2A receptor agonist is administered at a dose of about 400 mg.

In some embodiments, a 5-HT2A receptor agonist is mescaline. In some embodiments, a 5-HT2A receptor agonist is administered at a dose between about 50 mg and about 1,000 mg. In some embodiments, a 5-HT2A receptor agonist is administered at a dose selected from the group consisting of about 50 mg, about 100 mg, about 200 mg, about 300 mg, about 400 mg, about 500 mg, about 600 mg, about 700 mg, about 800 mg, about 900 mg, and about 1,000 mg. In some embodiments, a 5-HT2A receptor agonist is administered at a dose of about 50 mg. In some embodiments, a 5-HT2A receptor agonist is administered at a dose of about 100 mg. In some embodiments, a 5-HT2A receptor agonist is administered at a dose of about 200 mg. In some embodiments, a 5-HT2A receptor agonist is administered at a dose of about 300 mg. In some embodiments, a 5-HT2A receptor agonist is administered at a dose of about 400 mg. In some embodiments, a 5-HT2A receptor agonist is administered at a dose of about 500 mg. In some embodiments, a 5-HT2A receptor agonist is administered at a dose of about 600 mg. In some embodiments, a 5-HT2A receptor agonist is administered at a dose of about 700 mg. In some embodiments, a 5-HT2A receptor agonist is administered at a dose of about 800 mg. In some embodiments, a 5-HT2A receptor agonist is administered at a dose of about 900 mg. In some embodiments, a 5-HT2A receptor agonist is administered at a dose of about 1,000 mg.

In some embodiments, a 5-HT2A receptor agonist is 5-MeO-DMT. In some embodiments, 5-McO-DMT is administered at a dose between about 1.0 and about 30 mg. In some embodiments, 5-MeO-DMT is administered at a dose selected from the group consisting of about 1.0 mg, about 2.5mg, about 5.0 mg, about 6.0 mg, about 7.0 mg, about 8.0 mg, about 9.0 mg, about 10 mg, about 12.5 mg, about 15 mg, about 17.5 mg, about 20 mg, about 25 mg, and about 30 mg. In some embodiments, 5-MeO-DMT is administered at a dose of about 1.0 mg. In some embodiments, 5-MeO-DMT is administered at a dose of about 2.5 mg. In some embodiments, 5-MeO-DMT is administered at a dose of about 5.0 mg. In some embodiments, 5-MeO-DMT is administered at a dose of about 6.0 mg. In some embodiments, 5-MeO-DMT is administered at a dose of about 7.0 mg. In some embodiments, 5-MeO-DMT is administered at a dose of about 8.0 mg. In some embodiments, 5-MeO-DMT is administered at a dose of about 9.0 mg. In some embodiments, 5-MeO-DMT is administered at a dose of about 10 mg. In some embodiments, 5-MeO-DMT is administered at a dose of about 12.5 mg. In some embodiments, 5-MeO-DMT is administered at a dose of about 15 mg. In some embodiments, 5-MeO-DMT is administered at a dose of about 17.5 mg. In some embodiments, 5-MeO-DMT is administered at a dose of about 20 mg. In some embodiments, 5-MeO-DMT is administered at a dose of about 25 mg. In some embodiments, 5-McO-DMT is administered at a dose of about 30 mg.

In some embodiments, a 5-HT2A receptor agonist is DMT or MET. In some embodiments, DMT or MET is administered at a dose between about 5.0 and about 100 mg. In some embodiments, DMT or MET is administered at a dose selected from the group consisting of about 5.0 mg, about 10 mg, about 15 mg, about 20 mg, about 25 mg, about 30 mg, about 35 mg, about 40 mg, about 45 mg, about 50 mg, about 60 mg. about 70 mg, about 80 mg, about 90 mg, and about 100 mg. In some embodiments, DMT or MET is administered at a dose of about 5.0 mg. In some embodiments, DMT or MET is administered at a dose of about 10 mg. In some embodiments, DMT or MET is administered at a dose of about 15 mg. In some embodiments, DMT or MET is administered at a dose of about 20 mg. In some embodiments, DMT or MET is administered at a dose of about 25 mg. In some embodiments, DMT or MET is administered at a dose of about 30 mg. In some embodiments, DMT or MET is administered at a dose of about 35 mg. In some embodiments, DMT or MET is administered at a dose of about 40 mg. In some embodiments, DMT or MET is administered at a dose of about 45 mg. In some embodiments, DMT or MET is administered at a dose of about 50 mg. In some embodiments, DMT or MET is administered at a dose of about 60 mg. In some embodiments, DMT or MET is administered at a dose of about 70 mg. In some embodiments, DMT or MET is administered at a dose of about 80 mg. In some embodiments, DMT or MET is administered at a dose of about 90 mg. In some embodiments, DMT or MET is administered at a dose of about 100 mg.

In some embodiments, a 5-HT2A receptor agonist is selected from the group consisting of 25I-NBOMc, 25B-NBOMc, 25C-NBOMe, 25D-NBOMe, and 25E-NBOMe. In some embodiments, a 5-HT2A receptor agonist is administered at a dose between about 0.05 mg and about 2.0 mg. In some embodiments, a 5-HT2A receptor agonist is administered at a dose between about 0.05 mg, about 0.1 mg, about 0.2 mg, about 0.3 mg, about 0.4 mg, about 0.5 mg, about 0.75 mg, about 1.0 mg, about 1.25 mg, about 1.5 mg, and about 2.0 mg. In some embodiments, a 5-HT2A receptor agonist is administered at a dose of about 0.05 mg. In some embodiments, a 5-HT2A receptor agonist is administered at a dose of about 0.1 mg. In some embodiments, a 5-HT2A receptor agonist is administered at a dose of about 0.2 mg. In some embodiments, a 5-HT2A receptor agonist is administered at a dose of about 0.3 mg. In some embodiments, a 5-HT2A receptor agonist is administered at a dose of about 0.4 mg. In some embodiments, a 5-HT2A receptor agonist is administered at a dose of about 0.5 mg. In some embodiments, a 5-HT2A receptor agonist is administered at a dose of about 0.75 mg. In some embodiments, a 5-HT2A receptor agonist is administered at a dose of about 1.0 mg. In some embodiments, a 5-HT2A receptor agonist is administered at a dose of about 1.25 mg. In some embodiments, a 5-HT2A receptor agonist is administered at a dose of about 1.5 mg. In some embodiments, a 5-HT2A receptor agonist is administered at a dose of about 2.0 mg.

In some embodiments, a 5-HT2A receptor agonist is selected from the group consisting of 25I-NBOH, 25B-NBOH, 25C-NBOH, 25D-NBOH, 25E-NBOH, and 25CN-NBOH. In some embodiments, a 5-HT2A receptor agonist is administered at a dose between about 0.05 mg and about 3.0 mg. In some embodiments, a 5-HT2A receptor agonist is administered at a dose selected from the group consisting of about 0.050 mg, about 0.10 mg, about 0.20 mg, about 0.30 mg, about 0.40 mg, about 0.50 mg, about 0.75 mg, about 1.0 mg, about 1.25 mg, about 1.5 mg, about 2.0 mg, about 2.5 mg, and about 3.0 mg. In some embodiments, a 5-HT2A receptor agonist is administered at a dose of about 0.050 mg. In some embodiments, a 5-HT2A receptor agonist is administered at a dose of about 0.10 mg. In some embodiments, a 5-HT2A receptor agonist is administered at a dose of about 0.20 mg. In some embodiments. a 5-HT2A receptor agonist is administered at a dose of about 0.30 mg. In some embodiments, a 5-HT2A receptor agonist is administered at a dose of about 0.40 mg. In some embodiments, a 5-HT2A receptor agonist is administered at a dose of about 0.50 mg. In some embodiments, a 5-HT2A receptor agonist is administered at a dose of about 0.75 mg. In some embodiments, a 5-HT2A receptor agonist is administered at a dose of about 1.0 mg. In some embodiments, a 5-HT2A receptor agonist is administered at a dose of about 1.25 mg. In some embodiments, a 5-HT2A receptor agonist is administered at a dose of about 1.5 mg. In some embodiments, a 5-HT2A receptor agonist is administered at a dose of about 2.0 mg. In some embodiments, a 5-HT2A receptor agonist is administered at a dose of about 2.5 mg. In some embodiments, a 5-HT2A receptor agonist is administered at a dose of about 3.0 mg.

In an embodiment, the dosage of each of psilocybin, 4-AcO-DMT, or psilocin is about 10 to about 50 mg, PO. In another embodiment, the dosage of DMT is about 10 to about 50 mg, vaporized (inhaled) or IV. In another embodiment, the dosage of 5-MeO-DMT is about 5 to about 25 mg, vaporized (inhaled) or IV. In a further embodiment, the dosage of LSD is about 50 to about 300 ug, PO. In an embodiment, the dosage of mescaline is about 100 to about 500 mg, PO. In a further embodiment, the dosage of each of 2C-B, 2C-E, 2C-T-2, or 2C-T-7 is about 5 to about 30 mg, PO. In another embodiment, the dosage of DOM is about 2 to about 10 mg. PO.

In some embodiments, a 5-HT2A receptor agonist is administered parenterally. In some embodiments, a 5-HT2A receptor agonist is administered orally. In some embodiments, a 5-HT2A receptor agonist is administered sublingually or buccally. In some embodiments, a 5-HT2A receptor agonist is administered sublingually. In some embodiments, a 5-HT2A receptor agonist is administered buccally. In some embodiments, a 5-HT2A receptor agonist is administered intranasally. In some embodiments, a 5-HT2A receptor agonist is inhaled as a vapor.

Typically, dosages may be administered to a subject once, twice, three or four times daily, every other day, every three days, once weekly, twice monthly, once monthly, or 3-4 times yearly. In embodiments, a compound disclosed herein is administered to a subject once in the morning, or once in the evening. In embodiments, a compound disclosed herein is administered to a subject once in the morning, and once in the evening. In embodiments, a compound disclosed herein is administered to a subject three times a day (e.g., at breakfast, lunch, and dinner), at a dose, e.g., of 50 mg/administration (e.g., 150 mg/day).

In embodiments, a compound disclosed herein may be administered, e.g., via inhalation or orally, at specified intervals. For example, during treatment a patient may be administered a compound disclosed herein at intervals of every, e.g., 1 year, 6 months, 90 days, 60 days, 30 days, 14 days, 7 days, 3 days, 24 hours, 12 hours, 8 hours, 6 hours, 5 hours, 4 hours, 3 hours, 2.5 hours, 2.25 hours, 2 hours, 1.75 hours, 1.5 hours, 1.25 hours, 1 hour, 0.75 hour, 0.5 hour, or 0.25 hour.

In embodiments, the combination or a pharmaceutically acceptable salt thereof described in the present disclosure is administered to a patient under the supervision of a healthcare provider.

In embodiments, the combination of the peripheral serotonin receptor antagonist or pharmaceutically acceptable salt thereof and the 5-HT2A receptor agonist or pharmaceutically acceptable salt thereof of the present disclosure is administered to a patient under the supervision of a healthcare provider at a clinic specializing in the delivery of psychoactive treatments.

In some embodiments, the administration to a patient of a high dose under the supervision of a healthcare provider occurs periodically in order to maintain a therapeutic effect in the patient, e.g., every three days, twice weekly, once weekly, twice monthly, once monthly, thrice yearly, twice yearly, or once yearly.

In some embodiments, the combination of the peripheral serotonin receptor antagonist or pharmaceutically acceptable salt thereof and the 5-HT2A receptor agonist or pharmaceutically acceptable salt thereof of the present disclosure is administered by a patient on their own at home or otherwise away from the supervision of a healthcare provider.

In some embodiments, the administration by a patient of a low dose on their own occurs periodically in order to maintain a therapeutic effect in the patient, e.g., daily, every other day, every three days, twice weekly, once weekly, twice monthly, or once monthly.

In some embodiments, a method described herein further comprises administering a monoamine oxidase inhibitor (MAOI) along with the 5-HT2A receptor agonist and peripheral serotonin receptor antagonist.

In some embodiments, compounds disclosed herein may be administered in combination with one or more other antidepressant treatments, such as, tricyclic antidepressants, MAOIs, SSRIs, and double and triple uptake inhibitors and/or anxiolytic drugs for manufacturing a medicament for treating depression, anxiety, and/or other related diseases, including to provide relief from depression or anxiety and preventing recurrence of depression or anxiety. In some embodiments, therapeutics that may be used in combination with a compound of the present disclosure include, but are not limited to, Anafranil, Adapin, Aventyl, Elavil, Norpramin, Pamelor, Pertofrane, Sinequan, Surmontil, Tofranil, Vivactil, Parnate, Nardil, Marplan, Celexa, Lexapro, Luvox, Paxil, Prozac, Zoloft, Wellbutrin, Effexor, Remeron, Cymbalta, Desyrel (trazodone), and Ludiomill.

Described herein are methods and compositions for treating a mood disorder by administering to a patient in need thereof a compound disclosed herein. Also provided are pharmaceutical compositions that include a compound disclosed herein.

In embodiments, the methods and compositions may be used to treat a mood disorder including depressive disorders, e.g., major depressive disorder, persistent depressive disorder, postpartum depression, premenstrual dysphoric disorder, seasonal affective disorder, psychotic depression, disruptive mood dysregulation disorder, substance/medication-induced depressive disorder, and depressive disorder due to another medical condition.

In some embodiments, depression conditions include major depressive disorder and dysthymic disorder. In some embodiments, depression conditions develop under unique circumstances, including, but are not limited to, psychotic depression, postpartum depression, seasonal affective disorder (SAD), mood disorder, depressions caused by chronic medical conditions such as cancer or chronic pain, chemotherapy, chronic stress, post-traumatic stress disorders, and bipolar disorder (or manic-depressive disorder). In some embodiments, depression conditions that are expected to be treated according to this aspect of the present disclosure include, but are not limited to, major depressive disorder, dysthymic disorder, psychotic depression, postpartum depression, premenstrual syndrome, premenstrual dysphoric disorder, seasonal affective disorder (SAD), anxiety, mood disorder, depressions caused by chronic medical conditions such as cancer or chronic pain, chemotherapy, chronic stress, post-traumatic stress disorders, and bipolar disorder (or manic depressive disorder).

Also provided herein are methods of treating refractory depression, e.g., patients suffering from a depressive disorder that does not, and/or has not, responded to adequate courses of at least one, or at least two, other antidepressant compounds or therapeutics. For example, provided herein is a method of treating depression in a treatment resistant patient, comprising a) optionally identifying the patient as treatment resistant and b) administering an effective dose of a disclosed compound. As used herein “depressive disorder” encompasses refractory depression. In some embodiments, refractory depression occurs in patients suffering from depression who are resistant to standard pharmacological treatments, including tricyclic antidepressants, MAOIs, SSRIs, and double and triple uptake inhibitors and/or anxiolytic drugs, as well non-pharmacological treatments such as psychotherapy, electroconvulsive therapy, vagus nerve stimulation and/or transcranial magnetic stimulation. In some embodiments, a treatment-resistant patient may be identified as one who fails to experience alleviation of one or more symptoms of depression (e.g., persistent anxious or sad feelings, feelings of helplessness, hopelessness, pessimism) despite undergoing one or more standard pharmacological or non-pharmacological treatment. In certain embodiments, a treatment-resistant patient is one who fails to experience alleviation of one or more symptoms of depression despite undergoing treatment with two different antidepressant drugs. In other embodiments, a treatment-resistant patient is one who fails to experience alleviation of one or more symptoms of depression despite undergoing treatment with four different antidepressant drugs. In some embodiments, a treatment-resistant patient may also be identified as one who is unwilling or unable to tolerate the side effects of one or more standard pharmacological or non-pharmacological treatment.

In some embodiments, symptoms associated with depression include, but are not limited to, persistent anxious or sad feelings, feelings of helplessness, hopelessness, pessimism, and/or worthlessness, low energy, restlessness, irritability, fatigue, loss of interest in pleasurable activities or hobbies, excessive sleeping, overeating, appetite loss, insomnia, thoughts of suicide, or suicide attempts. In some embodiments, various symptoms associated with anxiety include fear, panic, heart palpitations, shortness of breath, fatigue, nausea, and headaches, among others. In addition, patients suffering from any form of depression often experience anxiety. It is expected that the methods of the present condition can be used to treat anxiety or any of the symptoms thereof. In some embodiments, presence, severity, frequency, and duration of symptoms of depression vary on a case-to-case basis.

In embodiments, the methods and compositions may be used to treat a mood disorder including bipolar and related disorders, e.g., bipolar I disorder, bipolar II disorder, cyclothymic disorder, substance/medication-induced bipolar and related disorder, and bipolar and related disorder due to another medical condition.

In embodiments, the methods and compositions may be used to treat a mood disorder including substance-related disorders, e.g., preventing a substance use craving, diminishing a substance use craving, and/or facilitating substance use cessation or withdrawal. Substance use disorders involve abuse of psychoactive compounds such as alcohol, caffeine, cannabis, inhalants, opioids, sedatives, hypnotics, anxiolytics, stimulants, nicotine, and tobacco. As used herein “substance” or “substances” are psychoactive compounds which can be addictive such as alcohol, caffeine, cannabis, hallucinogens, inhalants, opioids, sedatives, hypnotics, anxiolytics, stimulants, nicotine, and tobacco. For example, the methods and compositions may be used to facilitate smoking cessation or cessation of opioid use.

In embodiments, the methods and compositions may be used to treat a mood disorder including anxiety disorders, e.g., separation anxiety disorder, selective mutism, specific phobia, social anxiety disorder (social phobia), panic disorder, panic attack, agoraphobia, generalized anxiety disorder, substance/medication-induced anxiety disorder, and anxiety disorder due to another medical condition.

In embodiments, the methods and compositions may be used to treat a mood disorder including obsessive-compulsive and related disorders, e.g., obsessive-compulsive disorder, body dysmorphic disorder, hoarding disorder, trichotillomania (hair-pulling disorder), excoriation (skin-picking) disorder, substance/medication-induced obsessive-compulsive and related disorder, and obsessive-compulsive and related disorder due to another medical condition.

In embodiments, the methods and compositions may be used to treat a mood disorder including trauma- and stressor-related disorders, e.g., reactive attachment disorder, disinhibited social engagement disorder, posttraumatic stress disorder, acute stress disorder, and adjustment disorders.

In embodiments, the methods and compositions may be used to treat a mood disorder including feeding and eating disorders, e.g., anorexia nervosa, bulimia nervosa, binge-eating disorder, pica, rumination disorder, and avoidant/restrictive food intake disorder.

In embodiments, the methods and compositions may be used to treat a mood disorder including neurocognitive disorders, e.g., delirium, major neurocognitive disorder, mild neurocognitive disorder, major or mild neurocognitive disorder due to Alzheimer's disease, major or mild frontotemporal neurocognitive disorder, major or mild neurocognitive disorder with Lewy bodies, major or mild vascular neurocognitive disorder, major or mild neurocognitive disorder due to traumatic brain injury, substance/medication-induced major or mild neurocognitive disorder, major or mild neurocognitive disorder due to HIV infection, major or mild neurocognitive disorder due to prion disease, major or mild neurocognitive disorder due to Parkinson's disease, major or mild neurocognitive disorder due to Huntington's disease, major or mild neurocognitive disorder due to another medical condition, and major or mild neurocognitive disorder due to multiple etiologies.

In embodiments, the methods and compositions may be used to treat a mood disorder including neurodevelopmental disorders, e.g., autism spectrum disorder, attention-deficit/hyperactivity disorder, stereotypic movement disorder, tic disorders, Tourette's disorder, persistent (chronic) motor or vocal tic disorder, and provisional tic disorder. In some embodiments, a variety of other neurological conditions are expected to be treated according to the methods of the present disclosure. In some embodiments, neurological conditions include, but are not limited to, a learning disorder, autistic disorder, attention-deficit hyperactivity disorder, Tourette's syndrome, phobia, post-traumatic stress disorder, dementia, AIDS dementia, Alzheimer's disease, Parkinson's disease, spasticity, myoclonus, muscle spasm, bipolar disorder, a substance abuse disorder, urinary incontinence, and schizophrenia.

In embodiments, the methods and compositions may be used to treat a mood disorder including personality disorders, e.g., borderline personality disorder.

In embodiments, the methods and compositions may be used to treat a mood disorder including sexual dysfunctions, e.g., delayed ejaculation, erectile disorder, female orgasmic disorder, female sexual interest/arousal disorder, genito-pelvic pain/penetration disorder, male hypoactive sexual desire disorder, premature (early) ejaculation, and substance/medication-induced sexual dysfunction.

In embodiments, the methods and compositions may be used to treat a mood disorder including gender dysphoria.

The above examples are encompassed by the term “psychiatric disorder”, as used herein.

Suitable dosage forms for a compound disclosed herein include, but are not limited to, oral forms, such as tablets, hard or soft gelatin capsules, powders, granules and oral solutions, syrups or suspensions, troches, as well as sublingual, buccal, intratracheal, intraocular, or intranasal forms, forms adapted to inhalation, topical forms, transdermal forms, or parenteral forms, for example, forms adapted for intravenous, intra-arterial, intraperitoneal, intrathecal, intraventricular, intramuscular or subcutaneous administration. In embodiments, for such parenteral administration, it may be in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

The present disclosure provides either one pharmaceutical composition comprising a peripheral serotonin receptor antagonist and a central 5-HT2A receptor agonist or they may be present in separate pharmaceutical compositions. Regardless, the pharmaceutical compositions of the present disclosure also comprise a pharmaceutically acceptable carrier. Pharmaceutical compositions herein may be provided with immediate release, delayed release, extended release, or modified release profiles. In embodiments, pharmaceutical compositions with different drug release profiles may be combined to create a two-phase or three-phase release profile. For example, pharmaceutical compositions may be provided with an immediate release or an extended-release profile. In embodiments, pharmaceutical compositions may be provided with an extended release and delayed release profile. Such composition may be provided as pulsatile formulations, multilayer tablets, or capsules containing tablets, beads, granules, etc. Compositions may be prepared using a pharmaceutically acceptable “carrier” composed of materials that are considered safe and effective. The “carrier” includes all components present in the pharmaceutical formulation other than the active ingredient or ingredients. The term “carrier” includes, but is not limited to, diluents, binders, lubricants, glidants, disintegrants, fillers, and coating compositions.

Pharmaceutical compositions include those suitable for oral, rectal, nasal, topical (including transdermal, buccal, and sublingual), vaginal or parenteral (including subcutaneous, intramuscular, intravenous, intranasal, inhaled, and intradermal) administration or administration via an implant. The compositions may be prepared by any method well known in the art of pharmacy.

Such methods include the step of bringing in association compounds used in the disclosure or combinations thereof with any auxiliary agent. The auxiliary agent(s), also named accessory ingredient(s), include those conventional in the art, such as carriers, fillers, binders, diluents, disintegrants, lubricants, colorants, flavoring agents, antioxidants, and wetting agents. Such auxiliary agents are suitably selected with respect to the intended form and route of administration and as consistent with conventional pharmaceutical practices.

Pharmaceutical compositions suitable for oral administration may be presented as discrete dosage units such as pills, tablets, dragées or capsules, or as a powder or granules, or as a solution or suspension. The active ingredient may also be presented as a bolus or paste. The compositions can further be processed into a suppository or enema for rectal administration.

Tablets may contain the active ingredient compounds and suitable binders, lubricants, disintegrating agents, coloring agents, flavoring agents, flow-inducing agents, and melting agents. Gelatin capsules may contain the active ingredient compounds and powdered carriers, such as lactose, starch, cellulose derivatives, magnesium stearate, stearic acid, and the like. Similar diluents can be used to make compressed tablets. Compressed tablets can be sugar coated or film coated to mask any unpleasant taste and protect the tablet from the atmosphere, or enteric coated for selective disintegration in the gastrointestinal tract. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol, and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

For oral administration in liquid dosage form, the oral drug components are combined with any oral, non-toxic, pharmaceutically acceptable inert carrier such as ethanol, glycerol, water, and the like. Examples of suitable liquid dosage forms include, but are not limited to, solutions or suspensions in water, pharmaceutically acceptable fats, and oils, alcohols, or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Liquid dosage forms for oral administration can contain coloring and flavoring to increase patient acceptance.

For parenteral administration, suitable compositions include aqueous and non-aqueous sterile solutions. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water-soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl-or propyl-paraben, and chlorobutanol. The compositions may be presented in unit-dose or multi-dose containers, for example sealed vials and ampoules, and may be stored in a freeze-dried (lyophilized) condition requiring only the addition of sterile liquid carrier, for example water, prior to use. For transdermal administration, e.g., gels, patches or sprays can be contemplated. Compositions or formulations suitable for pulmonary administration e.g., by nasal inhalation, include fine dusts, mists, or vapors, which may be generated by means of metered dose pressurized aerosols, nebulizers, insufflators, or heating elements. Parenteral and intravenous forms may also include minerals and other materials to make them compatible with the type of injection or delivery system chosen.

The compounds used in the method of the present disclosure may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds used in the method of the present disclosure may also be coupled to soluble polymers as targetable drug carriers or as prodrugs. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Pharmaceutical compositions herein may be provided with immediate release, delayed release, extended release, or modified release profiles. In some embodiments, pharmaceutical compositions with different drug release profiles may be combined to create a two-phase or three-phase release profile. For example, pharmaceutical compositions may be provided with an immediate release and an extended-release profile. In some embodiments, pharmaceutical compositions may be provided with an extended release and delayed release profile. Such composition may be provided as pulsatile formulations, multilayer tablets, or capsules containing tablets, beads, granules, etc.

Pharmaceutical compositions herein may be provided with abuse deterrent features by techniques know in the art, for example, by making a tablet that is difficult to crush or to dissolve in water.

The disclosure further includes a pharmaceutical composition comprising the antagonist and agonist together in one pharmaceutical composition or two separate pharmaceutical compositions, one comprising the antagonist, and one comprising the agonist, as hereinbefore described, in combination with packaging material, including instructions for the use of the composition for a use as hereinbefore described. In an embodiment, the disclosure further includes a kit comprised of a pharmaceutical composition comprising the antagonist as the active ingredient in association with a first pharmaceutical carrier therefor, and a pharmaceutical composition comprising the agonist as the active ingredient in association with a second pharmaceutical carrier, which first pharmaceutical carrier may be the same as or different from the second pharmaceutical carrier, and instructions for use, as described herein.

The exact dose and regimen of administration of the composition will necessarily be dependent upon the type and magnitude of the therapeutic or nutritional effect to be achieved. The treatment regimen and the dosage are determined by the physician based upon various factors and may vary depending on factors such as the particular combination of peripheral serotonin receptor antagonist and central 5-HT2A receptor agonist, and the compound, formula, route of administration, or age and condition of the individual subject to whom the composition is to be administered.

The combination of peripheral serotonin receptor antagonist, and central 5-HT2A receptor agonist used in the method of the present disclosure may be administered in various forms, including those detailed herein. The treatment with the compound may be a component of a combination therapy or an adjunct therapy, i.e., the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

In an embodiment, the peripheral antagonist is intended to be used in combination with moderate to high doses of 5-HT2A agonist psychedelics, as provided herein, which typically result in unpleasant peripheral side effects (e.g., BP increase, vasoconstriction, nausea) along with their desired central/therapeutic effects. Such drugs are typically only given intermittently, e.g., weekly, monthly, or annually, since they have robust and durable therapeutic effects after a single dose. Sessions are usually conducted under physician supervision considering the profound hallucinogenic effects that are induced.

The peripheral antagonist is to be administered before or simultaneously with any therapeutic (or recreational) use of any 5-HT2A agonist psychedelic to reduce the unpleasant peripheral side effects (often called “body load” by recreational users), while retaining the desired central psychoactive (therapeutic) effects.

In general, the peripheral serotonin receptor antagonist (e.g., BW501C67 or xylamidine) should be given simultaneously (or approximately simultaneously) by the same route as the 5-HT2A receptor agonist, or alternatively, from about 30 minutes up to several hours, e.g. up to about 2hours before the agonist, but, in an embodiment, about 30 min to 1 hour before the agonist, if the antagonist is given orally. If given before the agonist, the antagonist does not necessarily need to be administered by the same route. In general, the relative time of administration and the route of administration for both the antagonist and the agonist should be selected so that the peripheral antagonist has time to be absorbed and occupy 5-HT2A receptors before or simultaneous with attempted occupancy of those same receptors by the agonist. For example, the agonist and antagonist might both be given simultaneously by the same route of administration, such as the oral or IV route, or the antagonist might be given orally about 1 hour before the agonist is given by IV. However, one would NOT want to give the antagonist orally simultaneously with the agonist IV, since the agonist would be absorbed effectively instantly, while the antagonist would be absorbed more slowly after oral administration, resulting in agonist-induced peripheral side effects before the antagonist took effect. Further, the peripheral antagonist ideally should NOT be given AFTER the agonist.

If given simultaneously, the peripheral antagonist and central agonist might be combined in a single dosage form, e.g., a tablet for oral administration or solution for injection containing both compounds at dosages prescribed elsewhere in this disclosure. For example, a tablet containing 10-50 mg of psilocybin and 2-20 mg of BW501C67, or a solution for IV injection containing 10-50 mg of DMT and 0.2-2 mg of BW501C67 per unit dose volume.

Generally, the minimal dose of peripheral antagonist should be used to achieve the desired degree of attenuation of the peripheral side effects of the 5-HT2A receptor agonist, since minimizing the dose will limit the possibility of partial blockade of the desired central serotonergic effects.

The described doses of antagonists, such as BW501C67 and/or analogs thereof, and xylamidine and/or analogs thereof, should work to substantially attenuate or completely eliminate the peripheral side effects of a typical fully psychedelic dose of any 5-HT2A receptor agonist. However, it should be understood that in the event of an overdose of a 5-HT2A receptor agonist, a higher dose of the peripheral antagonist might be needed to appropriately counteract the peripheral effects, consistent with well-understood concepts of competitive antagonism. Similarly, for very long-acting psychedelics, such as DOI or DOPR, higher doses of the peripheral antagonist might be needed to extend its duration of effect to appropriately match the duration of effect of the agonist, again consistent with well-understood principles of pharmacokinetics, where a higher dose of a drug typically maintains an effective plasma concentration of said drug for a longer period of time post administration.

### Definitions

Unless indicated to the contrary, the terms “antagonist”, “peripheral antagonist”, “5-HT2A antagonist”, “5-HT2A receptor antagonist”, “peripheral 5-HT2A receptor antagonist”, or “peripheral serotonin receptor antagonist” refer to compounds having peripherally restricted antagonist effects on the 5-HT2A receptor and optionally one or more other serotonin receptors. Moreover, unless indicated to the contrary, the terms “agonist”, “5-HT2A agonist”, “5-HT2A receptor agonist”, or “central 5-HT2A receptor agonist” refer to compounds having agonist action on 5-HT2A receptors and optionally one or more other serotonin receptors in the central nervous system.

The term “and/or” as used herein means that the listed items are present, or used, individually or in combination. In effect, this term means that “at least one of” or “one or more” of the listed items is used or present. The term “and/or” with respect to pharmaceutically acceptable salts, means that the compounds of the disclosure exist as individual salts and solvates, as well as a combination of, for example, salts of a compound of the disclosure.

As used in the present disclosure, the singular forms “a”, “an” and “the” include plural references unless the content clearly dictates otherwise. For example, an embodiment including “a compound” should be understood to present certain aspects with one compound, or two or more additional compounds.

Further, unless expressly stated to the contrary, “or” refers to an inclusive “or” and not to an exclusive “or.” For example, a condition A or B is satisfied by any one of the following: A is true (or present), and B is false (or not present); A is false (or not present), and B is true (or present); and both A and B are true (or present).

In the context of the present disclosure the term “5-HT2A receptor agonist” is intended to mean any compound or substance that activates the 5-HT2A receptor. The agonist may be a partial or full agonist.

The term “peripheral serotonin receptor antagonist” as used herein, means a compound that preferentially antagonizes 5-HT2A receptors, and optionally one or more additional serotonin receptor types, located outside the central nervous system.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments. aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl) alkyl, (cycloalkenyl) alkyl or (cycloalkyl) alkenyl.

The term “alkyl” refers to a straight or branched alkyl group. Exemplary alkyl groups are methyl, ethyl, propyl, isopropyl, butyl, isobutyl, and tert-butyl.

The term “haloalkyl” refers to a straight or branched alkyl group that is substituted with one or more halogen atoms.

The term “halogen” means F, Cl, Br, or I.

As used herein, the term “pharmaceutically acceptable” refers to molecular entities and compositions that are “generally regarded as safe”, e.g., that are physiologically tolerable and do not typically produce an allergic or similar untoward reaction when administered to a human. In embodiments, this term refers to molecular entities and compositions approved by a regulatory agency of the federal or a state government, as the GRAS list under sections 204(s) and 409 of the Federal Food, Drug and Cosmetic Act, that is subject to premarket review and approval by the FDA or similar lists, the U.S. Pharmacopeia or another generally recognized pharmacopeia for use in animals, and more particularly in humans.

As described herein. compounds of the invention may contain “optionally substituted” moieties. In general, the term “substituted” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group. the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R°; —(CH₂)₀₋₄R°; —O(CH₂)₀₋₄R°, —O—(CH₂)₀₋₄C(O)OR°; —(CH₂)₀₋₄CH(OR°)₂; —(CH₂)₀₋₄SR°; —(CH₂)₀₋₄Ph, which may be substituted with R°; —(CH₂)₀₋₄O(CH₂)₀₋₁Ph which may be substituted with R°; —CH═CHPh, which may be substituted with R°; —(CH₂)₀₋₄₀(CH₂)₀₋₁-pyridyl which may be substituted with R°; —NO₂; —CN; —N₃; —(CH₂)₀₋₄N(R°)₂; —(CH₂)₀₋₄N(R°)C(O)R°; N(R°)C(S)R°; —(CH₂)₀₋₄N(R°)C(O)NR°₂; —N(R°)C(S)NR°₂; —(CH₂)₀₋₄N(R°)C(O)OR°; —N(R°)N(R°)C(O)R°; —N(R°)N(R°)C(O)NR°₂; —N(R°)N(R°)C(O)OR°; —(CH₂)₀₋₄C(O)R°; —C(S)R°; —(CH₂)₀₋₄C(O)OR°; —(CH₂)₀₋₄C(O)SR°; —(CH₂)₀₋₄C(O)OSiR°₃; —(CH₂)₀₋₄OC(O)R°; —OC(O)(CH₂)₀₋₄SR°, SC(S)SR°; —(CH₂)₀₋₄SC(O)R°; —(CH₂)₀₋₄C(O)NR°₂; —C(S)NR°₂; —C(S)SR°; —SC(S)SR°, —(CH₂)₀₋₄C(O)NR°₂;—C(O)N(OR°)°; —C(O)C(O)R°; —C(O)CH₂C(O)R°; —C(NOR°)R°; —(CH₂)₀₋₄SSR°; —(CH₂)₀₋₄S(O)₂R°; —(CH₂)₀₋₄S(O)₂OR°; —(CH₂)₀₋₄S(O)₂R°; —S(O)₂NR°₂; —(CH₂)₀₋₄S(O)R°; —N(R°)S(O)₂NR°₂; —N(R°)S(O)₂R°; —N(OR°)R°; —C(NH)NR°₂; —P(O)₂R°; —P(O)R°₂; —OP(O)R°)₂; —OP(O)(OR°)₂; SiR°₃; —(C₁₋₄ straight or branched alkylene)O—N(R°)₂; —(C₁₋₄ straight or branched)alkylene)C(O)O—N(R°)₂, wherein each R° may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above. two independent occurrences of R°, taken together with their intervening atom(s), form a 3-12-membered saturated, partially unsaturated, or aryl mono-or bicyclic ring having 0-4 heteroatoms independently selected from nitrogen, oxygen, or sulfur, which may be substituted as defined below.

Suitable monovalent substituents on R° (or the ring formed by taking two independent occurrences of R° together with their intervening atoms), are independently halogen, —(CH₂)₀₋₂R^{●}, —(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSir^{●}₃, —C(O)SR^{●}, —(C₁₋₄ straight or branched alkylene) C(O) OR^{●}, or —SSR^{●} wherein each R° is unsubstituted or where preceded by “halo” is substituted only with one or more halogens, and is independently selected from C₁-C₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R° include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated. partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R⁵⁷⁴ , —(haloR^{●}), 13 OH, —OR^{●}, —O(haloR^{●}), —CN, —C(O)OH, —C(O)OR^{●}), —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R⁵⁵⁴, —NR⁵⁵⁴₂, —C(O)R⁵⁵⁴ , —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N (R^{†})S(O)₂R^{†}; wherein each R^{†} is independently hydrogen, C₁₋₆ aliphatic which may be substituted as defined below. unsubstituted —OPh, or an unsubstituted 5-6-membered saturated. partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono-or bicyclic ring having 0-4 heteroatoms independently selected from nitrogen, oxygen, or sulfur.

Suitable substituents on the aliphatic group of R^{†} are independently halogen, —R^{●}, —(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁-₄ aliphatic, —CH₂Ph, —O(CH₂)₀₋₁Ph, or a 5-6-membered saturated, partially unsaturated, or aryl ring having 0-4 heteroatoms independently selected from nitrogen. oxygen, or sulfur.

As used herein, the terms “subject” and “patient” refer to an animal (e.g., a mammal, such as a human). A subject to be treated according to the methods described herein may be one who has been diagnosed with a particular condition, or one at risk of developing such conditions. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

“Treatment” and “treating,” as used herein, refer to the medical management of a subject with the intent to improve, ameliorate, stabilize (i.e., not worsen), prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy). Treatment also includes diminishment of the extent of the disease or condition; preventing spread of the disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “pharmaceutically acceptable salts” includes both acid and base addition salts, wherein the compound is modified by making acid or base salts thereof. Examples of pharmaceutically acceptable salts include but are not limited to mineral or organic acid salts of basic residues such as amines, and alkali or organic salts of acidic residues such as carboxylic acids. Pharmaceutically acceptable salts include conventional non-toxic salts or quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, naphthalenesulfonic, methanesulfonic, ethane disulfonic, and oxalic acids. The pharmaceutically acceptable salts of a compound disclosed herein can be synthesized from the parent compound, which contains a basic or acidic moiety, by conventional chemical methods.

The terms “about” or “approximately” as used herein mean within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, a range up to 10%, a range up to 5%, and/or a range up to 1% of a given value. Alternatively, particularly with respect to biological systems or processes, the term can mean within an order of magnitude, e.g., within 5-fold, or within 2-fold, of a value. “About” and “approximately” are used interchangeably herein.

In embodiments, the terms “effective amount” or “therapeutically effective amount” refer to an amount of a compound, material, composition, medicament, or other material that is effective to achieve a particular pharmacological and/or physiologic effect including but not limited to reducing the frequency or severity of sadness or lethargy, depressed mood, anxious or sad feelings, diminished interest in all or nearly all activities, significant increased or decreased appetite leading to weight gain or weight loss, insomnia, irritability, fatigue, feelings of worthlessness, feelings of helplessness, inability to concentrate, and recurrent thoughts of death or suicide, or to provide a desired pharmacologic and/or physiologic effect, for example, reducing, inhibiting, or reversing one or more of the underlying pathophysiological mechanisms underlying the neurological dysfunction, modulating dopamine levels or signaling, modulating serotonin levels or signaling, modulating norepinephrine levels or signaling, modulating glutamate or GABA levels or signaling, modulating synaptic connectivity or neurogenesis in certain brain regions, or a combination thereof. The precise dosage will vary according to a variety of factors such as subject-dependent variables (e.g., age, immune system health, clinical symptoms etc.), the disease or disorder being treated. as well as the route of administration and the pharmacokinetics of the agent being administered.

Throughout the specification, dosage amounts are provided. The dosage amounts of the peripheral serotonin receptor antagonists and central 5-HT2A receptor agonists are found in different sections of the application. Nevertheless, the dosage amounts provided are for an average, approximately 70 kg adult human. In addition, the dosage amounts provided are the amounts of peripheral serotonin receptor antagonists and central 5-HT2A receptor agonists that are used in the combination therapy.

In embodiments, deuterium-enriched compounds disclosed herein, and their use is contemplated and within the scope of the methods and compositions described herein. Deuterium can be incorporated in any position in place of hydrogen (protium) synthetically, according to synthetic procedures known in the art. For example, deuterium may be incorporated to various positions having an exchangeable proton, such as an amine N—H, via proton-deuterium equilibrium exchange. Thus, deuterium may be incorporated selectively or non-selectively through methods known in the art.

In some embodiments, the level of deuterium at each deuterium-enriched —H site of the compound is 0.02% to 100%.

In some embodiments, the level of deuterium at each deuterium-enriched —H site of the compound is 50%-100%, 70%-100%, 90%-100%, 95%-100%, 96%-100%, 97%-100%, 98%-100%, or 99%-100%.

The compounds disclosed herein may be racemic and/or optically active isomers thereof. In this regard, some of the compounds can have asymmetric carbon atoms, and therefore, can exist either as racemic mixtures or as individual optical isomers (enantiomers). Compounds described herein that contain a chiral center include all possible stereoisomers of the compound, including compositions including the racemic mixture of the two enantiomers, as well as compositions including each enantiomer individually, substantially free of the other enantiomer. Thus, for example, contemplated herein is a composition including the S enantiomer of a compound substantially free of the R enantiomer, or the R enantiomer substantially free of the S enantiomer. If the named compound includes more than one chiral center, the scope of the present disclosure also includes compositions including mixtures of varying proportions between the diastereomers, as well as compositions including one or more diastereomers substantially free of one or more of the other diastereomers. By “substantially free” it is meant that the composition includes less than 25%, 15%, 10%, 8%, 5%, 3%, or less than 1% of the minor enantiomer or diastereomer(s).

Methods for synthesizing, isolating, preparing, and administering various stereoisomers are known in the art. Separation of diastereomers or cis and trans isomers may be achieved by conventional techniques, such as, for example, by fractional crystallization, chromatography, or High-Performance Liquid Chromatography (HPLC) of a stereoisomeric mixture of the agent or a suitable salt or derivative thereof. An individual enantiomer of a compound disclosed herein may also be prepared from a corresponding optically pure intermediate or by resolution, such as by HPLC of the corresponding racemate using a suitable chiral support or by fractional crystallization of the diastereomeric salts formed by reaction of the corresponding racemate with a suitable optically active acid or base, as appropriate.

The subject disclosure is also intended to include all isotopes of atoms occurring in the compounds disclosed herein. Isotopes include those atoms having the same atomic number but different mass numbers. By way of general example and without limitation, isotopes of hydrogen include tritium and deuterium. Isotopes of carbon include ¹³C and ¹⁴C.

It will be noted that any notation of a carbon in structures throughout this application, when used without further notation, is intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, is intended to represent all isotopes of hydrogen, such as ¹H, ²H, or ³H. Furthermore, any compounds containing ²H or ³H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

### EXEMPLIFICATION

The compounds used in the method of the present disclosure may be prepared by techniques well known in organic synthesis and familiar to a practitioner ordinarily skilled in the art. For example, the compounds may be prepared by the synthetic transformations shown below under general procedures and further described in the specific examples that follow.

### Abbreviations

ACN: Acetonitrile
4-HO-MET: 4-hydroxy-N-methyl-N-ethyltryptamine
2C-B: 4-bromo-2,5-dimethoxyphenethylamine
CDI: N,N′-carbonyldiimidazole
2C-E: 4-ethyl-2,5-dimethoxyphenethylamine
DCM: Dichloromethane
DIPEA: Diisopropylethylamine
DMF: N,N-dimethylformamide
DMAc: Dimethylacetamide
DMSO: Dimethylsulfoxide
DMT: N,N-dimethyltryptamine
25D-NBOMe: 2-(4-methyl-2,5-dimethoxyphenyl)-N-[(2-methoxyphenyl) methyl]ethanamine
DOI: 4-iodo-2,5-dimethoxyamphetamine
DOPR: 2,5-dimethoxy-4-propylamphetamine
5-F-MET: 5-fluoro-N-methyl-N-ethyltryptamine
HLM: human liver microsomes
4-HO-MET: 4-hydroxy-N-methyl-N-ethyltryptamine
HPLC: High-performance liquid chromatography
HRMS: High-resolution mass spectrometry
LCMS: Liquid chromatography-mass spectrometry
LC-MS/MS: Liquid chromatography-tandem mass spectrometry
LSD: lysergic acid diethylamide
MAO: monoamine oxidase
5-MeO-DMT: 5-methoxy-N,N-dimethyltryptamine
NADPH: Nicotinamide adenine dinucleotide phosphate hydride
NMP: N-methylpyrrolidone
NMR: Nuclear magnetic resonance
PBS: phosphate buffered saline
Pd/C: Palladium on carbon
RLM: rat liver microsomes
R.T.: Room temperature/ambient temperature
THF: Tetrahydrofuran
TLC: Thin-layer chromatography
T3P: Polyphosphoric anhydride

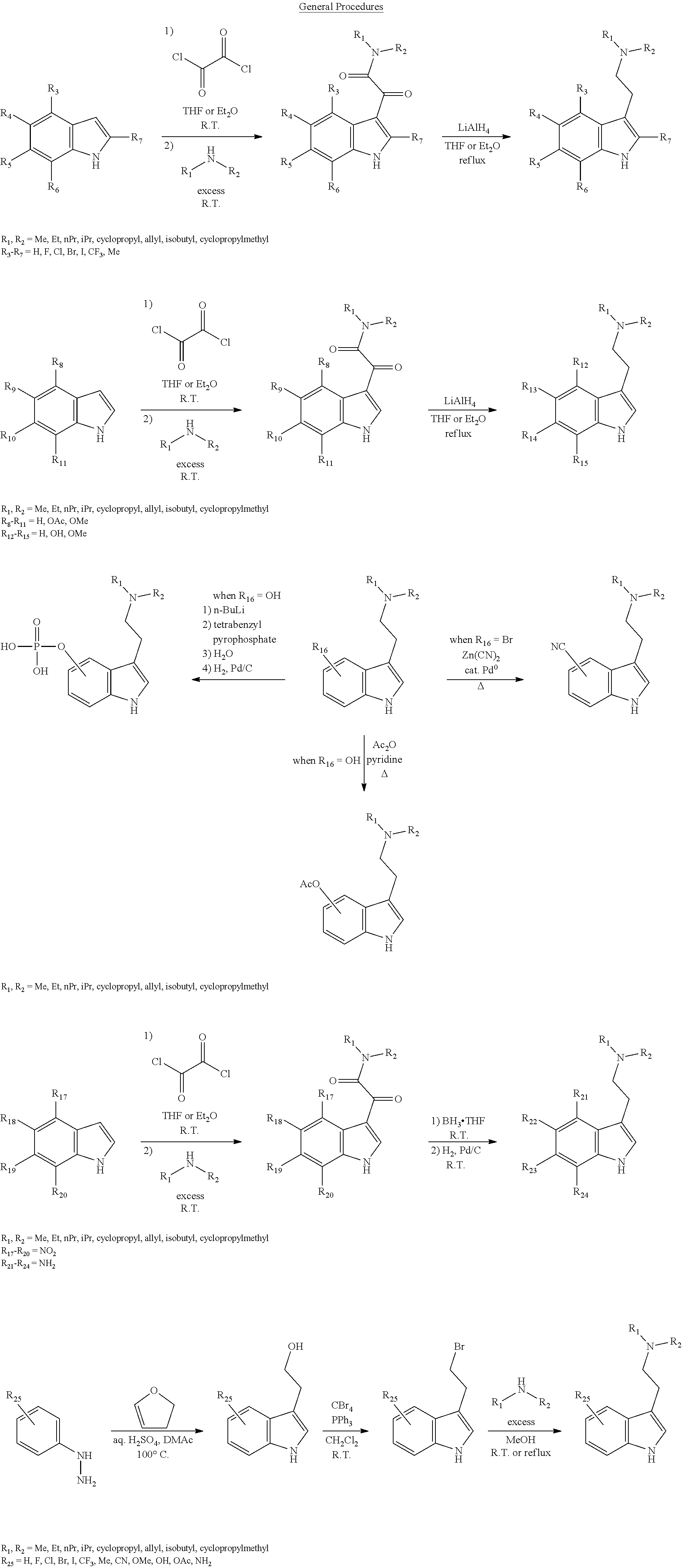

However, these may not be the only means by which to synthesize or obtain the desired compounds.

### EXAMPLES

### Example 1

### Preparation of BW501C67 Formate

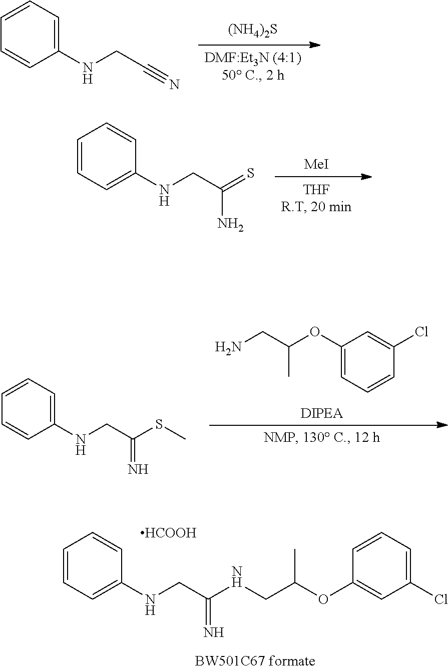

### Step 1: Preparation of 2-(phenylamino)ethanethioamide

To a stirred solution of 2-(phenylamino) acetonitrile (1.0 g, 7.57 mmol, 1 eq) in dimethylformamide (8.0 mL) was added triethylamine (2.0 mL) at room temperature. Then diammonium sulfide (1.03 g, 15.13 mmol, 2.0 eq) was added to the reaction mixture at 0° C. After complete addition, reaction mixture was stirred at room temperature for 5 min. The reaction mixture was then stirred at 50° C. for 2 h. The reaction progress was monitored by TLC and LCMS. After completion, the reaction mixture was quenched by water (30 mL). Solids were filtered and residual compound was dried over high vacuum to afford crude 2-(phenylamino) ethanethioamide as yellow solid. Crude compound was directly used for the next step without further purification. Yield: 1.0 g, 79.5%. HRMS m/z 167.15 [M+1]⁺.

### Step 2: Preparation of methyl 2-(phenylamino)ethanimidothioate

To a stirred solution of 2-(phenylamino) ethanethioamide (1 g, 6.02 mmol, 1.0 eq) dissolved in THF (10 mL) was added iodomethane (3.74 mL, 60.2 mmol, 10 eq) at 0° C. The reaction mixture was stirred at 0° C. for 10 min, then warmed room temperature and stirred for 20 min. Reaction progress was monitored by TLC and LCMS. After completion of the reaction, the mixture was concentrated under reduced pressure to afford crude methyl 2-(phenylamino)ethanimidothioate. Crude compound was directly used for the next step without further purification. Yield: 1.0 g, 88%. HRMS m/z 181.20 [M+1]⁺.

### Step 3: Preparation of N-(2-(3-chlorophenoxy)propyl)-2-(phenylamino)acetimidamide formate (BW501C67 formate)

To stirred solution of crude methyl 2-(phenylamino) ethanimidothioate (1.0 g, 5.55 mmol. 1.0 eq) dissolved in NMP (10 mL) was added ethylbis (propan-2-yl) amine (2.91 mL, 16.64 mmol, 3 eq), 2-(3-chlorophenoxy)propan-1-amine (514 mg, 2.77 mmol, 0.5 eq) at room temperature. Then the reaction mixture was heated to 130° C. and stirred for 12 h. The reaction progress was monitored by TLC and LCMS. After completion of the reaction, the mixture was concentrated under reduced pressure to afford a crude material that was purified by prep HPLC (water/acetonitrile gradient, 0.1% formic acid buffer in water). The desired fractions were lyophilized to afford N-(2-(3-chlorophenoxy) propyl)-2-(phenylamino)acetimidamide formate (BW501C67 formate) as an off-white solid. Yield: 290 mg, 16.2%. HRMS m/z 317.13 [M+1]⁺. ¹H NMR (400 MHz, DMSO-d₆): δ=8.45 (s, 1H), 7.31 (t, J=8.4 Hz, 1H), 7.02 (t, J=7.2 Hz, 1H), 6.91 (d, J=9.6 Hz, 1H), 6.62 (t, J=7.2 Hz, 1H), 6.47 (d, J=7.6 Hz, 1H), 6.23 (bs, 1H), 4.64-4.61 (m, 1H), 3.93 ((bs, 1H), 3.60-3.50 (m, 1H), 3.46-3.40 (m, 1H), 1.18 (d, J=5.6 Hz, 3H).

### Example 2: Preparation of Xylamidine Trifluoroacetate

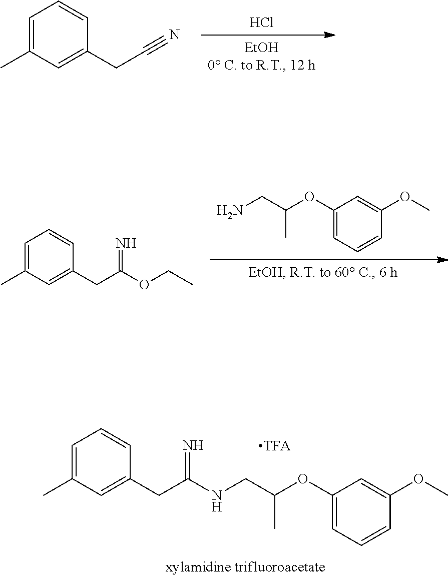

### Step 1: Preparation of ethyl 2-(m-tolyl) acetimidate

A stirred solution of 2-(m-tolyl) acetonitrile (2.0 g, 15.25 mmol, 1.0 eq) was dissolved in ethanol (20 mL), then cooled to 0° C. The solution was then sparged with dry HCl gas at 0° C. for 10 min. The reaction mixture was stirred at room temperature for 12 h and progress was monitored by TLC and LCMS. After completion of the reaction, the mixture was concentrated under reduced pressure by rotary evaporation at 45° C. to afford crude ethyl 2-(m-tolyl) acetimidate. Crude compound was directly used in the next step without any further purification. Yield: 1.60 g, 51.51%. HRMS m/z 178.30 [M+1]⁺.
Step 2: Preparation of N-(2-(3-methoxyphenoxy) propyl)-2-(m-tolyl) acetimidamide trifluoroacetate (xylamidine trifluoroacetate)

To a stirred solution of 2-(3-methoxyphenoxy) propan-1-amine (1.0 g, 5.64 mmol, 1.0 eq) in ethanol (20 mL) at room temperature was added ethyl 2-(m-tolyl) acetimidate (1.23 g, 6.77 mmol, 1.2 eq). Then the reaction mixture was heated and stirred at 60° C. for 6 h. Reaction progress was monitored by TLC and LCMS. After completion of the reaction, the mixture was cooled to room temperature and concentrated under reduced pressure by rotary evaporation at 45° C. to afford crude compound. This crude material was purified by prep HPLC (water/acetonitrile gradient, 0.1% trifluoroacetic acid buffer in water). The desired fractions were lyophilized to afford N-(2-(3-methoxyphenoxy) propyl)-2-(m-tolyl) acetimidamide trifluoroacetate (xylamidine trifluoroacetate) as a colorless, sticky liquid. Yield: 1.05 g, 60%. HRMS m/z 313.30 [M+1]⁺.

¹H NMR (400 MHZ, DMSO-d₆): δ=9.77 (t, J=4.8 Hz, 1H), 9.25 (s, 1H), 8.83 (s, 1H), 7.20-7.14 (m, 2H), 7.11-7.07 (m, 3H), 6.55 (dd, J=8.4 Hz, 2 Hz, 1H), 6.48 (dd, J=8.0 Hz, 2 Hz, 1H), 6.42 (t, J=2.4 Hz, 1H), 4.62-4.56 (m, 3H), 3.71 (s, 2H), 3.69 (s, 1H), 3.50 (t, J=6.4 Hz, 2H), 2.19 (s, 3H), 1.23 (d, J=6.0 Hz, 3H).

### Example 3: Preparation of 1-(4-fluorobenzyl)-3-(4-isobutoxybenzyl)-1-(piperidin-4-yl)urea hydrochloride (Compound 7) and(S)-2-amino-5-(4-(1-(4-fluorobenzyl)-3-(4-isobutoxybenzyl)ureido)piperidin-1-yl)-5-oxopentanoic acid (Compound 14a)

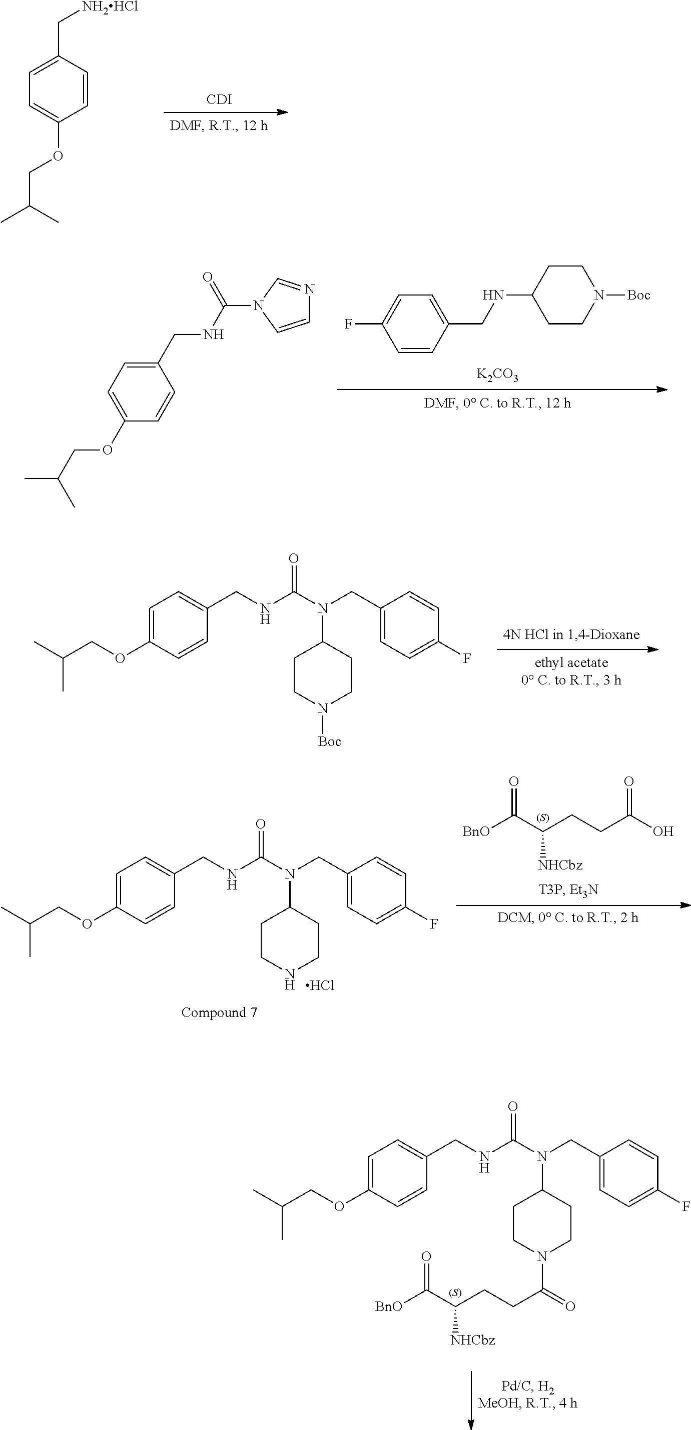

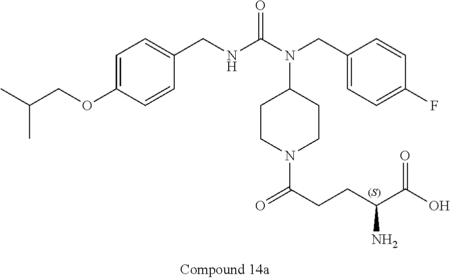

Step 1: Preparation of N-(4-isobutoxybenzyl)-1H-imidazole-1-carboxamide
To a stirred solution of (4-isobutoxyphenyl) methanamine hydrochloride (5.0 g, 23.18 mmol, 1.0 eq) in DMF (50 mL) was added N,N′-carbonyldiimidazole (4.02 g, 24.80 mmol, 1.07 eq) at room temperature and stirred at this temperature for 12 h. Reaction progress was monitored by TLC and LCMS. After completion of the reaction, the mixture was diluted with water extracted with ethyl acetate. The combined organic layers were washed with brine and dried over anhydrous Na₂SO₄, filtered, and solvent was evaporated under reduced pressure to afford crude material that was purified by column chromatography using 100-200 mesh silica. The compound eluted in 30% ethyl acetate in heptane. The desired fraction was concentrated under reduced pressure to afford N-(4-isobutoxybenzyl)-1H-imidazole-1-carboxamide. Yield: 4.50 g, 71.03%. HRMS m/z 514.05 [M+1]⁺.

### Step 2: Preparation of tert-butyl 4-(1-(4-fluorobenzyl)-3-(4-isobutoxybenzyl)ureido)piperidine-1-carboxylate

To a stirred solution of N-(4-isobutoxybenzyl)-1H-imidazole-1-carboxamide (4.50 g, 16.46 mmol, 1.0 eq) in dimethylformamide (50 mL) was added tert-butyl 4-{[(4-fluorophenyl)methyl]amino}piperidine-1-carboxylate (508 mg, 16.46 mmol, 1.0 eq), followed by potassium carbonate (3.41 g, 24.69 mmol, 1.5 eq) at room temperature. The reaction mixture was stirred at room temperature for 12 h and reaction progress was monitored by TLC and LCMS. After completion, the mixture was diluted with water extracted with ethyl acetate. The combined organic layers were washed with brine and dried over anhydrous Na₂SO₄, filtered and solvent was evaporated under reduced pressure to afford a crude compound. The crude compound was purified by column chromatography using 100-200 mesh silica, and the compound eluted in 2% methanol in DCM. The desired fraction was concentrated under reduced pressure to afford tert-butyl 4-(1-(4-fluorobenzyl)-3-(4-isobutoxybenzyl) ureido) piperidine-1-carboxylate. (Yield: 4.50 mg, 53%).

### HRMS m/z 514.25 [M+1]⁺.

### Step 3: Preparation of 1-(4-fluorobenzyl)-3-(4-isobutoxybenzyl)-1-(piperidin-4-yl)urea (Compound 7)

To a stirred solution of tert-butyl 4-(1-(4-fluorobenzyl)-3-(4-isobutoxybenzyl) ureido) piperidine-1-carboxylate (3.50 g, 6.81 mmol, 1 eq) in ethyl acetate (50 mL) at 0° C. was added 4 N HCl in 1,4-dioxane (25 mL) dropwise. The reaction mixture was then allowed to stir at room temperature for 3 h, and progress was monitored by LCMS. After completion, the mixture was concentrated under reduced pressure to afford a crude which was triturated with DCM (2.0 mL) and diethyl ether (20 mL), followed by n-pentane (30 mL). The resulting solid was dried under reduced pressure to afford 1-(4-fluorobenzyl)-3-(4-isobutoxybenzyl)-1-(piperidin-4-yl) urea hydrochloride (Compound 7) as an off-white solid. Yield: 3.0 g. 97%. HRMS m/z 414.30 [M+1]⁺. ¹H NMR (400 MHZ, MeOD): δ=7.26-7.23 (m, 2H), 7.10 (d, J=8 Hz, 2H), 7.07-7.02 (m, 2H), 6.82-6.79 (m, 2H), 4.49 (s, 2H), 4.34-4.27 (m, 3H), 3.70 (d, J=8 Hz, 2H), 3.41-3.38 (m, 2H), 3.07-3.01 (m, 2H), 2.06-2.00 (m,1H), 1.98-1.88 (m, 4H), 1.03-1.01 (m, 6H).

### Step 4: Preparation of benzyl(S)-2-(((benzyloxy)carbonyl)amino)-5-(4-(1-(4-fluorobenzyl)-3-(4-isobutoxybenzyl)ureido)piperidin-1-yl)-5-oxopentanoate

To a stirred solution of 1-(4-fluorobenzyl)-3-(4-isobutoxybenzyl)-1-(piperidin-4-yl)urea hydrochloride (Compound 7, 3.26 g. 7.25 mmol, 1.0 eq) and(S)-5-(benzyloxy)-4-(((benzyloxy)carbonyl)amino)-5-oxopentanoic acid (3.23 g, 8.71 mmol, 1.2 eq) in DCM (50 mL) was added triethylamine (3.03 mL, 21.8 mmol, 3.0 eq). Then the reaction mixture was cooled to 0° C. and propylphosphonic anhydride (50% solution in ethyl acetate, 6.48 mL, 10.9 mmol, 1.5 eq.) was added to the reaction mixture dropwise. The reaction mixture was then allowed to stir at room temperature for 2 hr. Reaction progress was monitored by TLC and LCMS. After completion of the reaction, the mixture was diluted with water and extracted with ethyl acetate. The combined organic layers were washed with brine and dried over anhydrous Na₂SO₄, filtered, and solvent was removed under reduced pressure to afford crude compound. This material was purified by column chromatography (100-200 mesh silica gel), and the compound was eluted in 15% ethyl acetate in heptane. Desired fractions were concentrated on rotary evaporator to afford benzyl(S)-2-(((benzyloxy) carbonyl) amino)-5-(4-(1-(4-fluorobenzyl)-3-(4-isobutoxybenzyl) ureido) piperidin-1-yl)-5-oxopentanoate as an off-white solid. Yield: 2.8 g, 50%. HRMS m/z 767.25 [M+1]⁺.

### Step 5: Preparation of(S)-2-amino-5-(4-(1-(4-fluorobenzyl)-3-(4-isobutoxybenzyl)ureido) piperidin-1-yl)-5-oxopentanoic acid (Compound 14a)

To a solution of benzyl(S)-2- (((benzyloxy) carbonyl) amino)-5-(4-(1-(4-fluorobenzyl)-3-(4-isobutoxybenzyl)ureido)piperidin-1-yl)-5-oxopentanoate (2.8 g. 3.65 mmol, 1 eq) in methanol (35 mL) was added Pd/C (1.8 g, 16.9 mmol) at room temperature. The reaction mixture was then stirred at room temperature under hydrogen atmosphere for 10 h using a hydrogen balloon. The reaction progress was monitored by TLC and LCMS. After completion of the reaction, the mixture was filtered through Celite, washed with methanol (200 mL), dried over anhydrous Na₂SO₄, and concentrated under reduced pressure on rotary evaporator at 45° C. (under inert atmosphere) to afford crude compound. This material was purified by trituration. Material was first dissolved in 5% methanol in DCM (3.0 mL), then added diethyl ether (15 mL) was added and stirred for 5 min. Solids were filtered dried to afford of(S)-2-amino-5-(4-(1-(4-fluorobenzyl)-3-(4-isobutoxybenzyl)ureido)piperidin-1-yl)-5-oxopentanoic acid (Compound 14a) as an off-white solid. Yield: 1.5 g, 75.71%. HRMS m/z 543.40 [M+1]⁺. ¹H NMR (400 MHZ, MeOD): δ=7.24-7.20 (m, 2H), 7.08 (d, J=12 Hz, 2H), 7.03-6.98 (m, 2H), 6.82-6.78 (m, 2H), 4.59-4.56 (m, 1H), 4.46 (s, 2H), 4.35-4.27 (m, 3H), 3.98-3.95 (m, 1H), 3.70 (d, J=8 Hz, 2H), 3.59-3.54 (m, 1H), 3.14-3.07 (m, 1H), 2.67-2.56 (m, 3H), 2.10-2.00 (m, 3H), 1.74-1.51 (m, 4H), 1.03 (d, J=6.4 Hz, 6H).

### Example 4

### Serotonin Receptor Binding

The binding affinities of key peripheral 5-HT2A receptor antagonists, as well as reference 5-HT2A agonists, at the ketanserin binding site of the 5-HT2A receptor and the mesulergine binding site of the 5-HT2C receptor were determined in radioligand binding experiments.

Method. Affinity of the test compounds for the 5-HT2A and 5-HT2C receptors was determined in radioligand binding experiments with [³H] ketanserin and [³H] mesulergine, respectively, by WuXi AppTec (Hong Kong) Limited, using methods adapted from the literature and under conditions described in Table 1.

TABLE 1
Assay conditions for radioligand binding assays.

| | 5-HT2A Binding | 5-HT2C Binding |
|---|---|---|
| Receptor Source | HEK293 stable | HEK293 stable |
| | cell line | cell line |
| Vehicle | 1.0% DMSO | 0.5% DMSO |
| Incubation Time | 1 h | 1 h |
| Incubation Temperature | 25° C. | 25° C. |
| Incubation Buffer | 50 mM Tris-HCl, | 50 mM Tris-HCl, |
| | pH 7.4 | pH 7.4 |
| Ligand | 1 nM [³H]ketanserin | 2 nM [³H]mesulergine |
| Non-Specific Ligand | 1 μM ketanserin | 1 μM SB 206553 |

Results. Results of the radioligand binding assays are shown in Table 2. All tested compounds were high-affinity ligands for 5-HT2A receptors, but xylamidine and BW501C67 were substantially more potent than Compound 14a. The affinities of the tested peripheral antagonists for this receptor were similar to or higher than those of several reference 5-HT2A agonists, suggesting good ability to block the effects of such agonists. Xylamidine and BW501C67 also had a high affinity for 5-HT2C receptors, whereas Compound 14a was selective for 5-HT2A versus 5-HT2C. Accordingly. xylamidine and BW501C67 may be useful to block both the 5-HT2A and 5-HT2C effects of serotonergic agonists active at both of these receptors (as are most classical psychedelics), whereas Compound 14a would be expected to leave the peripheral 5-HT2C component of such compounds intact.

TABLE 2
Results of receptor binding affinity experiments.

| | 5-HT2A Kᵢ (nM) | 5-HT2C Kᵢ (nM) |
|---|---|---|
| Compound | ([³H]ketanserin) | ([³H]mesulergine) |
| xylamidine | 0.55 | 1.45 |
| Compound 14a | 3.83 | 106.44 |
| BW501C67 | 0.42 | 1.34 |
| DOPR | 17.56 | 16.56 |
| DOI | 14.51 | 70.04 |
| 2C-B | 8.25 | 9.91 |
| 2C-E | 16.68 | 4.10 |
| 25D-NBOMe | 2.52 | NT |
| mescaline | 17,362 | >1,877 |
| DMT | 415.90 | NT |
| 5-F-MET | 140.03 | 59.00 |
| 4-HO-MET | 106.29 | NT |
| psilocin | 58.58 | NT |
| 5-MeO-DMT | 39.00 | NT |
| LSD | 0.89 | 6.45 |

NT = not tested

### Example 5

### Functional Activity at Serotonin Receptors

Key peripheral 5-HT2A receptors antagonists were tested for antagonist activity at several serotonin (5-HT) receptor subtypes (5-HT2A and 5-HT2B) using Ca²⁺ flux functional assays, with the results summarized in Table 3. All compounds exhibited potent antagonist activity at 5-HT2A receptors, but xylamidine and BW501C67 were substantially more potent than Compound 14a, consistent with their higher binding affinity for this receptor. Further, xylamidine and BW501C67showed high potency antagonism of 5-HT2B receptors, whereas Compound 14a did not. The ability to block peripheral 5-HT2B receptors in addition to 5-HT2A receptors is advantageous in the context of the present disclosure, as such blockade is likely to mitigate the potential cardiac valvulopathy induced by 5-HT2B receptor agonists. This is expected to be a potential side effect of many classical psychedelics, which activate 5-HT2B receptors as a secondary target. Accordingly, xylamidine and BW501C67 are preferred over Compound 14a for use in combination with 5-HT2A receptor agonists that also activate 5-HT2B receptors, since they also block this important off target. When taken together with the binding data presented above, xylamidine and BW501C67 appear to be potent pan-antagonists for 5-HT2-type receptors, whereas Compound 14a is selective for 5-HT2A receptors specifically.

Test Compounds. Compounds were commercially obtained or prepared as described above.

Functional Assays at 5-HT2A and 5-HT2B. Antagonist activity at 5-HT2A and 5-HT2B receptors was determined using a FLIPR Ca²⁺ flux assay at WuXi AppTec (Hong Kong) Limited according to their standard protocols. Briefly, stably transfected HEK293 cells expressing the receptor of interest were grown and plated in a 384 well plate and incubated at 37° C. and 5% CO₂ overnight. This was combined with a fluorescent dye (Fluo-4 Direct™) to make a final assay concentration of 2.5 mM. Compounds were diluted 1:3.16 for 10 points and 750 nL was added to a 384 well compound plate using ECHO along with 30 μL assay buffer. The fluorescent dye was then added to the assay plate along with assay buffer to a final volume of 40 μL. The cell plate was incubated for 50 min at 37° C. and 5% CO₂ and placed into the FLIPR Tetra along with the compound plate. 10 μL of references and compounds were then transferred from the compound plate into the cell plate and the fluorescent signal was read. Next, 10 μL of a 6× solution of 5-carboxamidotryptamine (5-CT) at the calculated EC₈₀ was added and the fluorescent signal read again. Finally, the Max-Min signal was calculated for the runs with and without agonist.

TABLE 3
Antagonist activity of compounds at select serotonin
receptors in Ca²⁺ flux functional assays.

| | 5-HT2A | 5-HT2A % | 5-HT2B | 5-HT2B % |
|---|---|---|---|---|
| | IC₅₀ | Inh @ | IC₅₀ | Inh @ |
| Compound | (nM) | Max Dose | (nM) | Max Dose |
| xylamidine | 23.97 | 99.81 | 13.27 | 99.68 |
| Compound 14a | 297.3 | 100.01 | >10,000 | −12.17 |
| BW501C67 | 14.51 | 100.13 | 21.64 | 99.74 |

### Example 6

### Effects of Peripheral Antagonists on the DOPR-Induced Head Twitch Response (HTR) in Rats

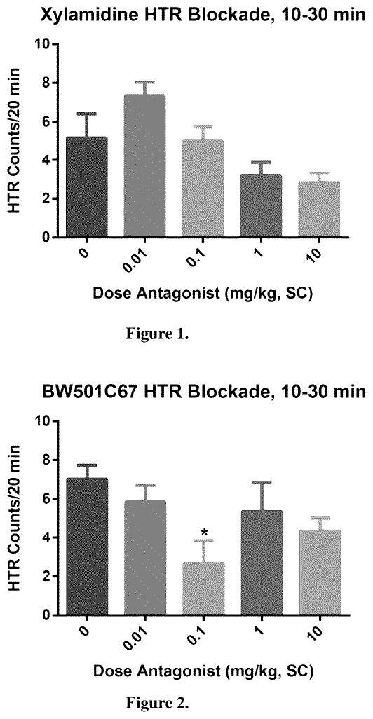

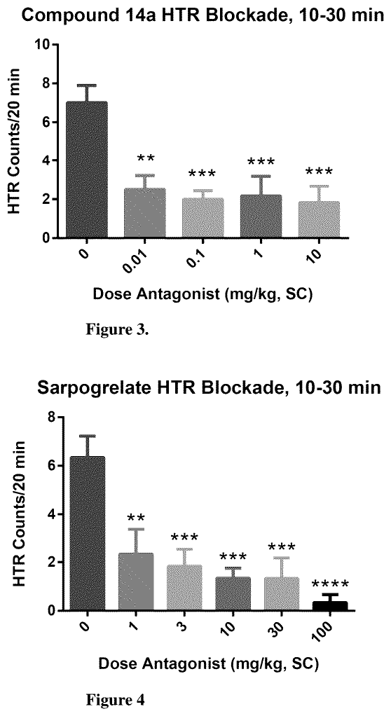

Key peripheral 5-HT2A receptor antagonists were tested for their ability to block the head twitch response (HTR) induced by a maximally effective dose of 2.5-dimethoxy-4-propylamphetamine (DOPR) in rats, with the results summarized in Table 4 and FIGS. 1-4. FIG. 1 graphically depicts in a bar graph the effect of xylamidine pre-treatment on the HTR induced by DOPR in the rat. The indicated doses of xylamidine or vehicle were administered SC. followed 10 minutes later by DOPR (0.32 mg/kg, SC), and HTRs were counted for 20 minutes immediately after DOPR administration (10-30 min after administration of xylamidine). Dunnett's multiple comparison test showed no significant effect (p>0.05) for any dose of xylamidine compared to vehicle. In FIG. 2, the effect of BW501C67 pre-treatment on the HTR induced by DOPR in the rat is depicted graphically in a bar graph. The indicated doses of BW501C67 or vehicle were administered SC, followed 10 minutes later by DOPR (0.32 mg/kg, SC), and HTRs were counted for 20 minutes immediately after DOPR administration (10-30 min after administration of BW501C67). *p<0.05 vs. vehicle using Dunnett's multiple comparison test; other groups not significant compared to vehicle (p>0.05). In FIG. 3, the effect of Compound 14a pre-treatment on the HTR induced by DOPR in the rat is depicted graphically in a bar graph. The indicated doses of Compound 14a or vehicle were administered SC, followed 10 minutes later by DOPR (0.32 mg/kg, SC), and HTRs were counted for 20 minutes immediately after DOPR administration (10-30min after administration of Compound 14a). **p<0.01. ***p<0.001 vs. vehicle using Dunnett's multiple comparison test. FIG. 4 graphically depicts in a bar graph the effect of sarpogrelate pre-treatment on the HTR induced by DOPR in the rat. The indicated doses of sarpogrelate or vehicle were administered SC, followed 10 minutes later by DOPR (0.32 mg/kg, SC), and HTRs were counted for 20 minutes immediately after DOPR administration (10-30 min after administration of sarpogrelate). **p<0.01, ***p<0.001, ****p<0.0001 vs. vehicle using Dunnett's multiple comparison test.

Although both xylamidine and BW501C67 numerically attenuated the HTR to DOPR, these effects were not statistically significant, with the exception of a single intermediate dose level of BW501C67 (and there was no clear dose-dependence to the effects of BW501C67). Accordingly. these compounds appear to show substantial peripheral restriction, as they were unable to fully block the centrally mediated HTR even at the highest dose tested. In contrast, both Compound 14a and sarpogrelate showed statistically significant attenuation of the HTR. even at low doses, suggesting that these compounds enter the brain in concentrations sufficient to interfere with the centrally mediated effects of 5-HT2A receptor agonists. Accordingly, of the compounds tested. only xylamidine and BW501C67 were good candidates for use in blocking the peripheral effects of 5-HT2A receptor agonists while avoiding blockade of their central effects.

Animals. Adult male Sprague-Dawley rats aged 8 weeks (body weight ˜270 g) were used in these experiments. Animals were pair housed under controlled temperatures and 12-hour light/dark cycles (lights on between 07:00-19:00 h), with ad libitum food and water. The protocol was approved by the Eurofins Advinus Institutional Animal Care and Use Committee. This study was carried out in strict accordance with the recommendations in the Guide for the Care and Use of Laboratory Animals of the National Institutes of Health. All efforts were made to minimize suffering.

Drugs and Drug Administration. Test compounds were prepared as described above or commercially obtained. Drugs were dissolved in their respective vehicle solutions (see Table 4) and administered subcutaneously (SC) in a volume of 5 mL/kg (except for the experiment with sarpogrelate, where a volume of 5 mL/kg was used). Test peripheral antagonists or vehicle were administered at 4-5 doses per compound (0.01 to 100 mg/kg, as indicated, calculated based on the free base), using N=6 animals/group. The 5-HT2A receptor agonist DOPR was then administered at 1 dose (0.32 mg/kg, SC, in saline, calculated based on the free base) to all animals 10 min after pretreatment with the test peripheral antagonist or vehicle.

Procedure. Rats were administered one dose (0.01 to 100 mg/kg, SC, as indicated) of a test peripheral antagonist (or vehicle) 10 mins prior to administration of DOPR (0.32 mg/kg, SC). After administration of DOPR, rats were immediately placed into a small open field for behavioral observation. Animals were observed continuously for 20 mins (10-30 mins after peripheral antagonist administration) and the number of HTRs were counted by an observer blind to the treatment condition.

Statistical analysis. The data points shown in Table 4 are the mean±standard error of the mean (SEM). Statistical comparisons were performed between each peripheral antagonist treatment group and its respective vehicle treatment group using Dunnett's multiple comparison test. Analysis was performed using GraphPad Prism 6.

TABLE 4
Blockade of DOPR-induced HTR by peripheral antagonists in rats.

| | | Mean HTR | |
|---|---|---|---|
| Compound | Dose (mg/kg) | (SEM) | |
| xylamidine | Vehicle (1% N- | 5.17 | (1.25) |
| | methylpyrrolidone | | |
| | in saline) | | |
| | 0.01 | 7.33 | (0.71) |
| | 0.1 | 5.00 | (0.73) |
| | 1 | 3.17 | (0.70) |
| | 10 | 2.83 | (0.48) |
| BW501C67 | Vehicle (1% | 7.00 | (0.73) |
| | DMSO in saline) | | |
| | 0.01 | 5.83 | (0.87) |
| | 0.1 | 2.67 | (1.17)* |
| | 1 | 5.33 | (1.52) |
| | 10 | 4.33 | (0.67) |
| Compound 14a | Vehicle | 7.00 | (0.89) |
| | (10% Captisol) | | |
| | 0.01 | 2.50 | (0.72)** |
| | 0.1 | 2.00 | (0.45)*** |
| | 1 | 2.17 | (1.01)*** |
| | 10 | 1.83 | (0.83)*** |
| sarpogrelate | Vehicle | 6.33 | (0.88) |
| | (10% DMSO, | | |
| | 40% PEG-300, | | |
| | 5% Tween-80, | | |
| | 45% saline) | | |
| | 1 | 2.33 | (1.02)** |
| | 3 | 1.83 | (0.70)*** |
| | 10 | 1.33 | (0.42)*** |
| | 30 | 1.33 | (0.84)*** |
| | 100 | 0.33 | (0.33)**** |

*p < 0.05,
**p < 0.01,
***p < 0.001,
****p < 0.0001 vs. vehicle using Dunnett's multiple comparison test

### Example 7

### Effect of Peripheral Antagonists on DOPR-Induced Blood Pressure Changes in Rats

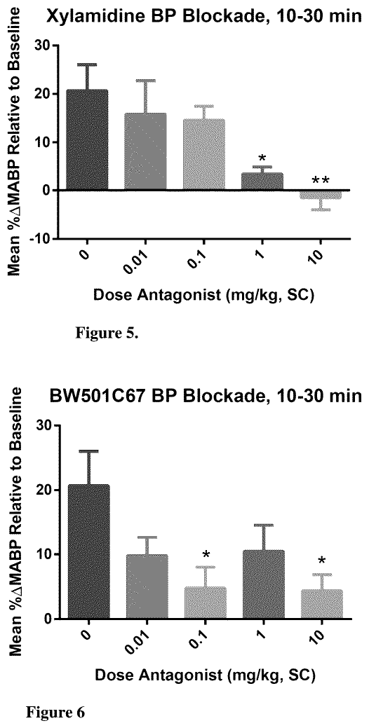

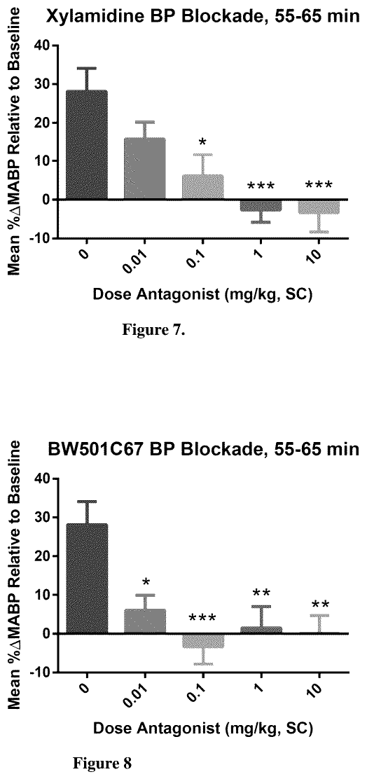

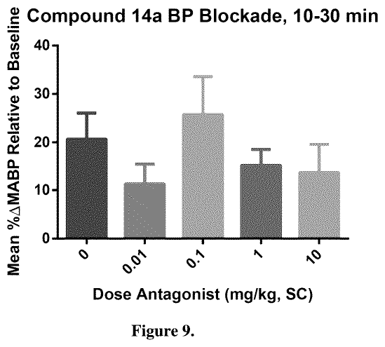

Key peripheral 5-HT2A receptor antagonists were tested for their ability to block the increases in blood pressure (BP) induced by a maximally effective dose (in terms of HTR response) of 2,5-dimethoxy-4-propylamphetamine (DOPR) in anesthetized rats, with the key results summarized in FIGS. 5-9. FIG. 5 depicts in a bar graph the effect of xylamidine pre-treatment on DOPR-induced elevation of MABP in the rat. The indicated doses of xylamidine or vehicle were administered SC, followed 10 minutes later by DOPR (0.32 mg/kg, SC), and mean percent change in MABP relative to the baseline recording for each group was calculated for the 20 minutes immediately after DOPR administration (10-30 min after administration of xylamidine). *p<0.05, **p<0.01 vs. vehicle using Dunnett's multiple comparison test. FIG. 6 depicts in a bar graph the effect of BW501C67 pre-treatment on DOPR-induced elevation of MABP in the rat. The indicated doses of BW501C67 or vehicle were administered SC, followed 10 minutes later by DOPR (0.32mg/kg, SC), and mean percent change in MABP relative to the baseline recording for each group was calculated for the 20 minutes immediately after DOPR administration (10-30 min after administration of BW501C67). *p<0.05 vs. vehicle using Dunnett's multiple comparison test. FIG. 7 depicts in a bar graph the effect of xylamidine pre-treatment on DOPR-induced elevation of MABP in the rat. The indicated doses of xylamidine or vehicle were administered SC. followed 10 minutes later by DOPR (0.32 mg/kg, SC), and mean percent change in MABP relative to the baseline recording for each group was calculated for the period 45-55 minutes after DOPR administration (55-65 min after administration of xylamidine). *p<0.05, ***p<0.001 vs. vehicle using Dunnett's multiple comparison test. FIG. 8 depicts in a bar graph the effect of BW501C67pre-treatment on DOPR-induced elevation of MABP in the rat. The indicated doses of BW501C67or vehicle were administered SC, followed 10 minutes later by DOPR (0.32 mg/kg, SC), and mean percent change in MABP relative to the baseline recording for each group was calculated for the period 45-55 minutes after DOPR administration (55-65 min after administration of BW501C67). *p<0.05, **p<0.01, ***p<0.001 vs. vehicle using Dunnett's multiple comparison test. FIG. 9 depicts in a bar graph the effect of Compound 14a pre-treatment on DOPR-induced elevation of MABP in the rat. The indicated doses of Compound 14a or vehicle were administered SC, followed 10 minutes later by DOPR (0.32 mg/kg, SC), and mean percent change in MABP relative to the baseline recording for each group was calculated for the 20 minutes immediately after DOPR administration (10-30 min after administration of Compound 14a). Dunnett's multiple comparison test showed no significant effect (p>0.05) for any dose of Compound 14a compared to vehicle.

In the 20 minutes immediately following administration of DOPR (10-30 mins after administration of the antagonist), both xylamidine (FIG. 5) and BW501C67 (FIG. 6) significantly attenuated the DOPR-induced elevation of mean arterial blood pressure (MABP) at intermediate to high doses. At 45-55 minutes after administration of DOPR (55-65 minutes after administration of the antagonist), which was near the time of maximal elevation in MABP in the vehicle-treated group, this inhibitory effect was even more pronounced, with both xylamidine (FIG. 7) and BW501C67 (FIG. 8) showing activity at lower doses. In contrast, Compound 14a failed to significantly attenuate the DOPR-induced elevation of MABP at any dose tested (FIG. 9), suggesting it is not effective as an agent for attenuating the peripheral effects of 5-HT2A receptor agonists.

Animals. Adult male Sprague-Dawley rats aged 8 weeks (body weight ˜270 g) were used in these experiments. Animals were pair housed under controlled temperatures and 12-hour light/dark cycles (lights on between 07:00-19:00 h), with ad libitum food and water. The protocol was approved by the Eurofins Advinus Institutional Animal Care and Use Committee. This study was carried out in strict accordance with the recommendations in the Guide for the Care and Use of Laboratory Animals of the National Institutes of Health. All efforts were made to minimize suffering.

Drugs and Drug administration. Test compounds were prepared as described above or commercially obtained. Drugs were dissolved in their respective vehicle solutions (xylamidine-1% N-methylpyrrolidone in saline; BW501C67-1% DMSO in saline: Compound 14a-10% Captisol) and administered subcutaneously (SC) in a volume of 10 mL/kg. Test peripheral antagonists or vehicle (saline) were administered at 4 doses per compound (0.01 to 10 mg/kg, as indicated, calculated based on the free base), using N=6 animals/group. The 5-HT2A receptor agonist DOPR was then administered at 1 dose (0.32 mg/kg. SC, in saline, calculated based on the free base) to all animals 10 min after pretreatment with the test peripheral antagonist or vehicle (saline).

Procedure. Rats were anesthetized using isoflurane and a catheter connected to a blood pressure transducer was inserted into the carotid artery. While maintained under anesthesia, animals were allowed 30 mins for their BP readings to stabilize. Following that, baseline recordings were taken for 30 mins prior to SC administration of the test peripheral antagonist or vehicle. 10 mins after administration of the test peripheral antagonist, all rats were administered DOPR (0.32 mg/kg, SC) and blood pressure and heart rate readings were recorded for an additional 60 mins. The following parameters were recorded throughout the 100 min session: systolic BP (sBP), diastolic BP (dBP), mean arterial blood pressure (MABP), and heart rate (HR).

Statistical Analysis. The data points shown in FIGS. 5-9 are the mean±standard error of the mean (SEM). Statistical comparisons were performed between each peripheral antagonist treatment group and the vehicle group using Dunnett's multiple comparison test. Analysis was performed using GraphPad Prism 6.

### Example 8

### Rat Plasma Protein Binding

Various putative peripheral 5-HT2A receptor antagonists were tested for plasma protein binding in rat plasma via equilibrium dialysis, with the results summarized in Table 5. A major metabolite of sarpogrelate, sarpogrelate M1, and a putative metabolite of Compound 14a, Compound 7, were also tested in this assay. The results were used to calculate the free brain: free plasma ratio for each compound in order to determine which compounds were most peripherally restricted (see Example 15).

Method 1. Aliquots of 50 μL loading matrix containing test compound (2 μM final incubation concentration) or control compound in triplicate were transferred to a Sample Collection Plate. The samples were matched with opposite blank buffer to obtain a final volume of 100 μL, with a volume ratio of matrix: Dialysis Buffer of 1:1 (v:v) in each well. Stop solution (200 ng/ml tolbutamide and 200 ng/mL labetalol in ACN) was added to these TO samples of test compound and control compound. The plate was sealed and shaken at 800 rpm for 10 min. An aliquot of 100 μL of the loading matrix containing test compound or control compound was transferred to the donor side of each dialysis well in triplicate and 100 μL of the Dialysis Buffer was loaded to the receiver side of the well. Then the plate was rotated at approximately 100 rpm in a humidified incubator with 5% CO₂ at 37±1° C. for 4 hours. At the end of the dialysis, aliquots of 50 μL samples from the buffer side and matrix side of the dialysis device were taken into new 96-well plates (Sample Collection Plates). An equal volume of opposite blank matrix (buffer or matrix) in each sample was added to reach a final volume of 100 μL, with volume ratio of matrix: Dialysis Buffer of 1:1 (v:v) in each well. All samples were further processed by protein precipitation before LC-MS/MS analysis.

Method 2. Aliquots (200 μL) of positive controls and test compounds at 5 μM (triplicates) were separately added to the plasma chamber and 350 L of phosphate buffered saline (pH 7.4) was added to the buffer chamber of the inserts. After sealing the rapid equilibrium dialysis (RED) device with an adhesive film, dialysis was performed in an incubator at 37° C. with shaking at 100 RPM for 4 hours. Aliquots (50 μL) of warfarin and test compounds were added to four 0.5 mL micro fuse tubes. Two aliquots were frozen immediately (0-minute sample). The other two aliquots were incubated at 37° C. for 4 hours along with the RED device. Following dialysis, an aliquot of 50 μL was removed from each well (both plasma and buffer side) and diluted with an equal volume of opposite matrix (dialyzed with the other matrix) to nullify the matrix effect. Similarly, 50 μL of buffer was added to recovery and stability samples. An aliquot of 100 μL was used for LC-MS/MS analysis following protein precipitation with ACN containing internal standard.

TABLE 5
Rat plasma protein binding of various
peripheral antagonists and metabolites.

| Compound | Mean % bound |
|---|---|
| BW501C67* | 89.3 |
| naftidrofuryl | unstable under |
| | assay conditions |
| temanogrel | 90.9 |
| sarpogrelate | 99.8 |
| sarpogrelate M1 | 97.2 |
| xylamidine | 65.6 |
| Compound 14a | 94.0 |
| Compound 7 | 96.2 |

*Analyzed via Method 1, all others via Method 2

### Example 9

### Rat Plasma Stability

Various putative peripheral 5-HT2A receptor antagonists were tested for rat plasma stability, with the results summarized in Table 6. A major metabolite of sarpogrelate, sarpogrelate M1, and a putative metabolite of Compound 14a, Compound 7, were also tested in this assay. The results were used to confirm that the plasma protein binding results were not confounded by poor plasma stability. Very low stability was observed for naftidrofuryl, consistent with its poor stability in the plasma protein binding study.

Method 1. Pooled frozen rat plasma was thawed in a water bath at 37° C. prior to the experiment. Plasma was centrifuged at 4,000 rpm for 5 min and any clots were remove. Using an Apricot automation workstation, 98 μL/well of blank plasma was added to all 96-well reaction plates (Blank, T0, T10, T30, T60 and T120). The Apricot automation workstation was used to add 2μL/well of working solution of test compounds (100 μM) to all reaction plates except Blank (T0, T10, T30, T60 and T120) to give a final incubation concentration of 2 μM. All reaction plates containing mixtures of compound and plasma were incubated at 37°° C. in a water bath for up to 120minutes. At the end of incubation, 500 μL of stop solution (200 ng/mL tolbutamide and 200 ng/ml labetalol in ACN) was added to precipitate protein and mixed thoroughly for 20 minutes at 4° C. After centrifugation, an Apricot automation workstation was used to transfer 150 μL of supernatant from each reaction plate to its corresponding bioanaylsis plate, which was shaken for 10 minutes prior to LC-MS/MS analysis.

Method 2. Positive controls and test compounds were spiked into 500 μL aliquots (n=3) of rat plasma and the resulting mixtures (5 μM final incubation concentration) were incubated at 37° C. An aliquot of 50 μL was withdrawn at 0, 15, 30, 60, 120, and 240 min and immediately quenched with 500 μL of ice-cold acetonitrile containing internal standard and the resulting samples were stored at minus 2-8° C. Samples were vortexed for 5 min followed by centrifugation at 4,000 RPM for 15 min at 4° C. An aliquot (100 μL) of each sample was transferred to a 96-well deep plate and submitted for analysis by LC-MS/MS.

TABLE 6
Rat plasma stability of various peripheral
antagonists and metabolites.

| | % Remaining |
|---|---|
| Compound | (120 minutes) |
| BW501C67* | 100.7 |
| naftidrofuryl | 0 |
| temanogrel | 95 |
| sarpogrelate | 67 |
| sarpogrelate M1 | 94 |
| xylamidine | 94 |
| Compound 14a | 97 |
| Compound 7 | 96 |

*Analyzed via Method 1, all others analyzed via Method 2

### Example 10

### Rat Brain Tissue Binding

Various putative peripheral 5-HT2A receptor antagonists were tested for binding to rat brain homogenate via equilibrium dialysis, with the results summarized in Table 7. A major metabolite of sarpogrelate, sarpogrelate M1, and a putative metabolite of Compound 14a, Compound 7, were also tested in this assay. The results were used to calculate the free brain:free plasma ratio for each compound in order to determine which compounds were most peripherally restricted (see Example 15). The results for naftidrofuryl, sarpogrelate, sarpogrelate M1, Compound 14A, and Compound 7 were potentially confounded by poor compound recovery or stability under the experimental conditions.

Method 1. Rat brain homogenate was thawed in a water bath at room temperature and then incubated at 37° C. for 10 min before use. Aliquots of 50 μL of loading matrix containing test compound (2 μM final incubation concentration) or control compound in triplicate were transferred to a Sample Collection Plate. The samples were matched with opposite blank buffer to obtain a final volume of 100 μL with a volume ratio of matrix: Dialysis Buffer of 1:1 (v:v) in each well. The stop solution (200 ng/mL tolbutamide and 200 ng/ml labetalol in ACN) was immediately added to these T0 samples of test compound and control compound. The plate was sealed and shaken at 800 rpm for 10 min. T0 samples were stored at 2-8° C. pending further processing along with other post-dialysis samples. An aliquot of 100 μL of the loading matrix containing test compound or control compound was transferred to the donor side of each dialysis well in triplicate and 100 μL of the Dialysis Buffer was loaded to the receiver side of the well. Then the plate was rotated at approximately 100 rpm in a humidified incubator with 5% CO₂ at 37±1° C. for 4 hours. At the end of the dialysis. aliquots of 50 μL from the buffer side and matrix side of the dialysis device were taken into new 96-well plates (Sample Collection Plates). An equal volume of opposite blank matrix (buffer or matrix) in each sample was added to reach a final volume of 100 μL, with a volume ratio of matrix: Dialysis Buffer of 1:1 (v:v) in each well. All samples were further processed by protein precipitation before LC-MS/MS analysis.

Method 2. Aliquots (200 μL) of test compounds and positive controls at 5 M in rat brain homogenate (triplicates) were added to the rapid equilibrium dialysis (RED) chamber of dialysis inserts. A 350 μL aliquot of dialysis buffer was added to the buffer chamber of the inserts. After sealing the RED device with an adhesive film, dialysis was conducted at 37° C. with shaking at 100 RPM for 4 hours. Following dialysis, an aliquot of 50 μL was removed from each well (brain homogenate and buffer) and diluted with an equal volume of opposite matrix to nullify the matrix effect. Similarly, buffer was added to recovery and stability samples. An aliquot of 100 μL was used for LC-MS/MS analysis following protein precipitation with ACN containing internal standard.

TABLE 7
Rat brain homogenate binding of various
peripheral antagonists and metabolites.

| | Mean % |
|---|---|
| Compound | bound |
| BW501C67* | 99.7 |
| Naftidrofuryl# | 96.1 |
| Temanogrel | 89.2 |
| Sarpogrelate** | 94.6 |
| Sarpogrelate M1# | 97.6 |
| Xylamidine | 93.3 |
| Compound 14a** | 92.2 |
| Compound 7# | 99.8 |

*Analyzed via Method 1, all others by Method 2;
**Compound stability <50% in matrix, so results may be unreliable;
#Compound recovery <50%, so results may be unreliable

### Example 11

### Rat Brain Tissue Stability

Various putative peripheral 5-HT2A receptor antagonists were tested for stability in rat brain homogenate, with the results summarized in Table 8. A major metabolite of sarpogrelate, sarpogrelate M1, and a putative metabolite of Compound 14a, Compound 7, were also tested in this assay. The results were used to confirm that the brain homogenate binding results were not confounded by poor brain tissue stability. Poor stability was observed for sarpogrelate and Compound 14a, consistent with the low stability of these compounds observed in the brain homogenate binding assay.

Method 1. Pooled frozen rat brain homogenate was thawed in a water bath at 37° C. prior to the experiment. Using an Apricot automation workstation, 98 μL/well of blank brain homogenate or 100 mM PBS were added to all 96-well reaction plates (Blank, T0, T10, T30, T60, and T120). The Apricot automation workstation was used to add 2 μL/well of working solution (50 μM) to all reaction plates except Blank (T0, T10, T30, T60, and T120) to give a final incubation concentration for test compounds of 1 μM. All reaction plates containing mixtures of compound and brain homogenate were incubated at 37° C. in a water bath for up to 120 minutes. At the end of incubation, 500 uL of stop solution (200 ng/mL tolbutamide and 200 ng/mL labetalol in ACN) was added to precipitate protein and mixed thoroughly for 20 minutes at 4° C. After centrifugation at 4,000 rpm and 4° C. for 20 minutes, an Apricot automation workstation was used to transfer 150 μL of supernatant from each reaction plate to its corresponding bioanaylsis plate, which was shaken for 10 minutes prior to LC-MS/MS analysis.

Method 2. Rat brain tissue homogenate samples were prepared by diluting one volume of whole rat brain tissue with two volumes of dialysis buffer (phosphate buffered saline pH 7.4-0.1 M sodium phosphate and 0.15 M sodium chloride) to yield 3 times diluted homogenate. Test compounds were spiked into 400 μL aliquots (n=3) of rat brain homogenate and the resulting mixtures were incubated at 37° C. An aliquot of 50 μL was withdrawn at 0, 15, 30, 60, 120, and 240min and immediately quenched with 400 μL of ice-cold acetonitrile containing internal standard and the resulting samples were stored at minus 2-8° C. After warming to room temperature, samples were vortexed for 5 min followed by centrifugation at 4,000 RPM for 15 min at 4° C. An aliquot (100 μL) of each sample was transferred to a 96-well deep plate and analyzed by LC-MS/MS.

TABLE 8
Rat brain homogenate binding of various
peripheral antagonists and metabolites.

| | % Remaining |
|---|---|
| Compound | (120 minutes) |
| BW501C67* | 110.8 |
| naftidrofuryl | 79 |
| temanogrel | 101 |
| sarpogrelate | 35 |
| sarpogrelate M1 | 84 |
| xylamidine | 100 |
| Compound 14a | 21 |
| Compound 7 | 69 |

*Analyzed via Method 1, all others analyzed via Method 2

### Example 12

### Metabolic Stability in Rat Liver Microsomes

Key peripheral 5-HT2A receptor antagonists were tested for stability in rat liver microsomes (RLM), with the results summarized in Table 9. Xylamidine and BW501C67 showed poor metabolic stability in RLM, indicating they likely exhibit low oral bioavailability. In contrast, Compound 14a was highly stable in RLM, suggestive of good oral bioavailability.

Test Compounds. Compounds were prepared as described above.

RLM Stability. Pooled RLM from adult male and female donors (Xenotech R1000) were used. Microsomal incubations were carried out in multi-well plates. Liver microsomal incubation medium consisted of PBS (100 mM, pH 7.4), MgCl₂ (1 mM), and NADPH (1 mM), with 0.50 mg of liver microsomal protein per mL. Control incubations were performed by replacing the NADPH-cofactor system with PBS. Test compounds (1 μM, final solvent concentration 1.0%) were incubated with microsomes at 37° C. with constant shaking. Six time points over 60 minutes were analyzed, with 60 μL aliquots of the reaction mixture being drawn at each time point. The reaction aliquots were stopped by adding 180 μL of cold (4° C.) acetonitrile containing 200 ng/mL tolbutamide and 200 ng/ml labetalol as internal standards (IS), followed by shaking for 10 minutes, and then protein sedimentation by centrifugation at 4,000 rpm for 20 minutes at 4° C. Supernatant samples (80 μL) were diluted with water (240 μL) and analyzed for parent compound remaining using a fit-for-purpose liquid chromatography-tandem mass spectrometry (LC-MS/MS) method.

Data Analysis. The elimination constant (kₑₗ), half-life (t_{1/2}), and intrinsic clearance (CLᵢₙₜ) were determined in a plot of ln(AUC) versus time, using linear regression analysis.

TABLE 9
Intrinsic clearance (CLᵢₙₜ) and half-life
(t_{1/2}) of compounds in the presence of RLM.

| | Clᵢₙₜ | t_{1/2} |
|---|---|---|
| Compound | (μL/min/mg) | (min) |
| xylamidine | 709.1 | 2.0 |
| Compound 14a | <9.6 | >145 |
| BW501C67 | 658.5 | 2.1 |

### Example 13

### Metabolic Stability in Human Liver Microsomes

Key peripheral 5-HT2A receptor antagonists were tested for stability in human liver microsomes (HLM), with the results summarized in Table 10. Xylamidine and BW501C67 showed poor metabolic stability in HLM, indicating they likely exhibit low oral bioavailability. In contrast, Compound 14a was highly stable in HLM, suggestive of good oral bioavailability.

Test Compounds. Compounds were prepared as described above.

HLM Stability. Pooled HLM from adult male and female donors (Corning 452117) were used. Microsomal incubations were carried out in multi-well plates. Liver microsomal incubation medium consisted of PBS (100 mM, pH 7.4), MgCl₂ (1 mM), and NADPH (1 mM), with 0.50 mg of liver microsomal protein per mL. Control incubations were performed by replacing the NADPH-cofactor system with PBS. Test compounds (1 μM, final solvent concentration 1.0%) were incubated with microsomes at 37° C. with constant shaking. Six time points over 60 minutes were analyzed, with 60 μL aliquots of the reaction mixture being drawn at each time point. The reaction aliquots were stopped by adding 180 μL of cold (4° C.) acetonitrile containing 200 ng/mL tolbutamide and 200 ng/ml labetalol as internal standards (IS), followed by shaking for 10 minutes, and then protein sedimentation by centrifugation at 4,000 rpm for 20 minutes at 4° C. Supernatant samples (80 μL) were diluted with water (240 μL) and analyzed for parent compound remaining using a fit-for-purpose liquid chromatography-tandem mass spectrometry (LC-MS/MS) method.

Data Analysis. The elimination constant (kₑₗ), half-life (t_{1/2}), and intrinsic clearance (CLᵢₙₜ) were determined in a plot of ln(AUC) versus time, using linear regression analysis.

TABLE 10
Intrinsic clearance (CLᵢₙₜ) and half-life
(t_{1/2}) of compounds in the presence of HLM.

| | Clᵢₙₜ | t_{1/2} |
|---|---|---|
| Compound | (μL/min/mg) | (min) |
| xylamidine | 448.6 | 3.1 |
| Compound 14a | <9.6 | >145 |
| BW501C67 | 192.4 | 7.2 |

### Example 14

### Pharmacokinetics in Rats

The pharmacokinetics of peripheral 5-HT2A antagonists were studied in the plasma (Tables 11 and 12) and brains (Table 13 and 14) of rats after subcutaneous (SC) dosing. The results were used to interpret the in vivo pharmacological results. Importantly, for the compounds tested in the HTR and BP studies, the plasma and brain concentrations across the most important time window of 10-30 min post antagonist administration were relatively stable, confirming that the pharmacological results were not confounded by a rapidly rising or falling concentration of the antagonist. Further, the longer half-life of BW501C67 suggests that it would be the best of the peripheral antagonists tested for use in combination with longer-acting 5-HT2A agonists, for example, LSD or DOM, where a longer duration of peripheral blockade would be required. Lastly, the PK results were also used for the calculation of free brain:free plasma ratios for each compound to determine the degree of peripheral restriction (see Example 15).

Animals. Male Sprague Dawley rats, aged 8-12 weeks. were used in these studies. Four rats were housed in each cage. Temperature and humidity were maintained at 22±3° C. and 30-70%, respectively, and illumination was controlled to give a 12 h light and 12 h dark cycle. Temperature and humidity were recorded by an auto-controlled data logger system. All animals were provided laboratory rodent diet and were fasted for 4 h pre-dose and 2 h post-dose. Reverse osmosis water treated with ultraviolet light was provided ad libitum. Animals were randomly assigned to treatment groups.

Drugs and Drug Administration. Test compounds were prepared as described above or commercially obtained. They were administered subcutaneously (SC) at a dose of 1 or 10 mg/kg (as indicated, calculated based on the free base) and at a volume of 5 mL/kg body weight. Vehicles were as follows: xylamidine—0.5% N-methylpyrrolidone (NMP) in normal saline; temanogrel—5% NMP, 5% Solutol HS-15, and 90% normal saline; sarpogrelate and naftidrofuryl—normal saline; Compound 14a—10% captisol; BW501C67—1% DMSO in normal saline.

Sample Collection and Bioanalysis. Blood samples (approximately 120 μL) were collected under light isoflurane anesthesia (Surgivet®) from the retro orbital plexus at 0.167, 0.5, 1, 2, 4, 8, and 24 h (4 animals per time point) for xylamidine, BW501C67, and Compound 14a after a dose of 1 mg/kg. SC. In separate experiments, plasma was harvested at 0.167 and 0.5 h (4 animals per time point) for naftidrofuryl, sarpogrelate. termanogrel, and Compound 14a after a dose of 10 mg/kg, SC. In these latter studies, sarpogrelate M1 was quantified in samples as a metabolite of sarpogrelate, and Compound 7 was quantified in samples as a putative metabolite of Compound 14a. For both studies, immediately after blood collection, plasma was harvested by centrifugation at 4,000 rpm for 10 min at 4° C. and samples were stored at −70±10° C. until bioanalysis. Following blood collection, animals were immediately sacrificed, the abdominal vena-cava was cut open, and the whole body was perfused from the heart using 10 mL of normal saline, and brain samples were collected from all animals. After isolation, brain samples were rinsed three times in ice-cold normal saline (for 5-10 seconds/rinse using ˜5-10 mL normal saline in disposable petri dish for each rinse) and dried on blotting paper. Brain samples were homogenized using ice-cold phosphate-buffered saline (pH 7.4). Total homogenate volume was three times the tissue weight. All homogenates were stored at −70±10° C. until bioanalysis. For bioanalysis, 25 μL aliquots of plasma/brain study samples or spiked plasma/brain calibration standards were added to individual pre-labeled micro-centrifuge tubes followed by 100 μL of an internal standard solution (glipizide, 500 ng/ml in acetonitrile) except for blanks, where 100 μL of acetonitrile was added. Samples were vortexed for 5 minutes and then centrifuged for 10 minutes at 4,000 rpm at 4° C. Following centrifugation, 100 μL of each clear supernatant was transferred to a 96-well plate and analyzed with a fit-for-purpose LC-MS/MS method, with authentic samples of each analyte used for calibration and identification.

Data Analysis. Pharmacokinetic parameters were estimated for Compound 14a, xylamidine, and BW501C67 after the 1 mg/kg dose using the non-compartmental analysis tool of Phoenix® WinNonlin software (Ver 8.0).

TABLE 11
Selected pharmacokinetic parameters of compounds in
plasma of Sprague Dawley rats (1 mg/k, SC dose).

| | | | | | | 0.167 | 0.5 |
|---|---|---|---|---|---|---|---|
| | | | | | | h | h |
| | | Tₘₐₓ | Cₘₐₓ | AUC₀₋ₗₐₛₜ | t_{1/2} | (ng/ | (ng/ |
| Compound | Route | (h) | (ng/mL) | (h*ng/mL) | (h) | mL) | mL) |
| Compound 14a | SC | 0.5 | 271.1 | 433.8 | 0.85 | 265.3 | 271.1 |
| xylamidine | SC | 0.17 | 155.4 | 343.6 | 1.71 | 155.4 | 133.4 |
| BW501C67 | SC | 0.17 | 91.36 | 277.2 | 3.11 | 91.4 | 73.8 |

TABLE 12
Mean plasma concentrations at 0.167
and 0.5 h (10 mg/kg, SC dose).

| | | 0.167 h | 0.5 h |
|---|---|---|---|
| Compound | Route | (ng/mL) | (ng/mL) |
| xylamidine | SC | 1099.2 | 670.0 |
| naftidrofuryl | SC | 6.43# | 4.38# |
| sarpogrelate | SC | 2130.3 | 2993.7 |
| sarpogrelate M1* | NA | 54.1 | 63.6 |
| temanogrel | SC | 410.6 | 518.3 |
| Compound 14a | SC | 965.6 | 1526.5 |
| Compound 7** | NA | 7.15 | 8.72 |

*Analyzed as a metabolite of sarpogrelate;
**Analyzed as a metabolite of Compound 14a;
#Plasma concentrations likely lower than reality due to poor plasma stability and decomposition during sample collection and processing;
NA = not applicable because only analyzed as metabolite

TABLE 13
Selected pharmacokinetic parameters of compounds in
brains of Sprague Dawley rats (1 mg/kg, SC dose).

| | | | | | | 0.167 | 0.5 |
|---|---|---|---|---|---|---|---|
| | | | | | | h | h |
| | | Tₘₐₓ | Cₘₐₓ | AUC₀₋ₗₐₛₜ | t_{1/2} | (ng/ | (ng/ |
| Compound | Route | (h) | (ng/g) | (h*ng/mL) | (h) | g) | g) |
| Compound 14a | SC | No quantifiable concentrations observed | | | | | |
| xylamidine | SC | 1 | 42.7 | 218.2 | 4.81 | 14.4 | 35.4 |
| BW501C67 | SC | 4 | 32.3 | 436.18 | NC | 10.4 | 13.6 |

NC = not calculated

TABLE 14
Mean brain concentrations at 0.167 and 0.5 h (10 mg/kg, SC dose).

| | | 0.167 h | 0.5 h |
|---|---|---|---|
| Compound | Route | (ng/g) | (ng/g) |
| xylamidine | SC | 100.8 | 60.7 |
| naftidrofuryl | SC | 679.2 | 688.86 |
| sarpogrelate | SC | 28.9 | 31.8 |
| sarpogrelate M1* | NA | 32.7 | 97.4 |
| temanogrel | SC | 112.9 | 147.6 |
| Compound 14a | SC | 35.63 | 40.69 |
| Compound 7** | NA | BLQ | BLQ |

*Analyzed as a metabolite of sarpogrelate;
**Analyzed as a metabolite of Compound 14a;
BLQ = below limit of quantitation;
NA = not applicable because only analyzed as metabolite

### Example 15

### Calculation of Free Brain: Free Plasma Ratio in Rats

The ratio of free brain concentration to free plasma concentration was calculated at 0.167 h and 0.5 h post administration for each compound using the data presented in the above examples and the following formula.

$$Free Brain : Free Plasma = ( Total Brain Concentration * Brain Free Fraction ) / ( Total Plasma Concentration * Plasma Free Fraction )$$

A smaller value for the free brain: free plasma ratio indicates greater peripheral restriction and therefore, better suitability for the methods of the present disclosure.

The results of these calculations are shown in Table 15. Among the compounds where values could be determined, BW501C67 was by far the most peripherally restricted compound, followed by xylamidine, suggesting these compounds are among the best candidates for use in the methods of the present disclosure. This is also supported by the above HTR and BP experiments, where these compounds more substantially blocked BP increases than HTR. Compound 14a also showed good peripheral restriction, however, the above HTR and BP experiments did not support this compound as a viable candidate for use in the methods of the present disclosure, since it substantially blocked HTR while having no effect on BP increases. Interestingly, temanogrel, sarpogrelate, and sarpogrelate's major metabolite, sarpogrelate M1, all showed substantial brain penetration and thus, despite claims to the contrary in the literature, do not appear to be well restricted to the periphery. This finding was in agreement with the robust ability of sarpogrelate to block the HTR. Accordingly, sarpogrelate and temanogrel are not suitable for use in the methods of the present disclosure. The ratio for naftidrofuryl could not be determined due to its poor plasma stability, but the high brain concentrations seen in PK suggest that it is not sufficiently restricted to the periphery.

TABLE 15
Free brain:free plasma ratios for putative peripheral antagonists. Values
calculated using 10 mg/kg PK data unless otherwise indicated.

| | Free Brain:Free | Free Brain:Free |
|---|---|---|
| Compound | Plasma (0.167 h) | Plasma (0.5 h) |
| naftidrofuryl | ND | ND |
| temanogrel | 0.33 | 0.34 |
| sarpogrelate# | 0.37 | 0.29 |
| sarpogrelate M1*# | 0.52 | 1.31 |
| xylamidine | 0.018 | 0.018 |
| compound 14a# | 0.048 | 0.035 |
| compound 7*# | 0** | 0** |
| xylamidine | 0.018 | 0.052 |
| (1 mg/kg data) | | |
| Compound 14a | 0** | 0** |
| (1 mg/kg data)# | | |
| BW501C67 | 0.0032 | 0.0052 |
| (1 mg/kg data) | | |

ND = not determined due to poor plasma stability;
*potential active metabolites quantified as metabolites in PK studies of sarpogrelate and Compound 14a, respectively;
**brain concentrations < lower limit of quantitation;
#values potentially confounded due to poor stability and/or recovery in brain tissue binding experiments

It should be understood that the examples and embodiments provided herein are exemplary. Those skilled in the art will envision various modifications of the examples and embodiments that are consistent with the scope of the disclosure herein. Such modifications are intended to be encompassed by the claims.

1. A method of treating a psychiatric disorder in a patient in need thereof, comprising administering to the patient a therapeutically effective amount of a combination of a peripheral serotonin receptor antagonist and a 5-HT2A receptor agonist, wherein the peripheral serotonin receptor antagonist and the 5-HT2A receptor agonist are present in one pharmaceutical composition comprising the peripheral serotonin receptor antagonist and the 5-HT2A receptor agonist and a pharmaceutically acceptable carrier therefor, or wherein the peripheral serotonin receptor antagonist and the 5-HT2A receptor agonist are present in two separate pharmaceutical compositions, wherein one pharmaceutical composition comprises the peripheral serotonin receptor antagonist and a pharmaceutically acceptable carrier therefor and the second pharmaceutical composition comprises the 5-HT2A receptor agonist and a pharmaceutically acceptable carrier therefor.

2. (canceled)

3. (canceled)

4. The method of claim 1, wherein the peripheral serotonin receptor antagonist and the 5-HT2A receptor agonist are in two separate pharmaceutical compositions and wherein the pharmaceutical composition comprising the peripheral serotonin receptor antagonist is administered sufficiently in advance of the pharmaceutical composition comprising the 5-HT2A receptor agonist to allow the peripheral serotonin receptor antagonist to substantially attenuate the peripheral side effects of the dose of the 5-HT2A receptor agonist.

5. The method of claim 1, wherein the peripheral serotonin receptor antagonist and the 5-HT2A receptor agonist are in two separate pharmaceutical compositions and wherein the pharmaceutical composition comprising the peripheral serotonin receptor antagonist is administered at least 30 minutes prior to the administration of the 5-HT2A receptor agonist.

6. The method of claim 1, wherein the peripheral serotonin receptor antagonist and the 5-HT2A receptor agonist are in two separate pharmaceutical compositions and wherein the pharmaceutical composition comprising the peripheral serotonin receptor antagonist and the pharmaceutical composition comprising the 5-HT2A receptor agonist are administered by the same route of administration.

7. The method of claim 1, wherein the peripheral serotonin receptor antagonist is administered once or twice or three times daily.

8. The method of claim 1, wherein the peripheral serotonin receptor antagonist is selected from the group consisting of xylamidine, an analog of xylamidine, BW501C67, and an analog of BW501C67, wherein the xylamidine analog has the formula R¹-A¹-NH—C(═NH)-A²-R², or a pharmaceutically acceptable acid addition salt thereof, wherein R¹ and R², which may be the same or different, are cach a phenyl or thien-2-yl group, optionally substituted in one or more positions by a halogen atom and/or a lower alkyl and/or a lower alkoxy and/or a hydroxy and/or a lower alkylthio and/or a trifluoromethyl and/or a phenyl and/or a phenoxy and/or a phenyl-(lower alkyl) and/or a phenyl-(lower alkoxy) group, each of said phenyl, phenoxy, phenyl-(lower alkyl) and phenyl-(lower alkoxy) groups being optionally substituted in one or more positions by a halogen atom and/or a lower alkyl and/or a lower alkoxy and/or a hydroxy and/or a lower alkylthio group; A¹ is a divalent straight or branched (oxy/thio)-alkylene linkage containing from two to six carbon atoms and optionally one or two divalent oxygen and/or sulfur atom(s), provided that there are at least two carbon atoms between the divalent atom and the —NH— group and between the two divalent atoms; A² is a straight or branched alkylene chain containing from one to four carbon atoms; and wherein in the definitions of R¹ and R², the term lower as applied to alkyl, alkoxy, or alkylthio groups or the alkyl, alkoxy, or alkylthio moicties of a group means an alkyl, alkoxy, or alkylthio group having 1 to 4 carbon atoms, and wherein the BW501C67 analog has the formula R¹⁰-A¹⁰-NH—C(═NH)-A²⁰-NZ-R²⁰, or a pharmaceutically acceptable acid addition salt thereof, wherein R¹⁰ and R²⁰, which may be the same or different, are each a phenyl or thien-2-yl group, optionally substituted in one or more positions by a halogen atom and/or a lower alkyl and/or a lower alkoxy and/or a hydroxy and/or a lower alkylthio and/or a trifluoromethyl and/or a phenyl and/or a phenoxy and/or a phenyl-(lower alkyl) and/or a phenyl-(lower alkoxy) group, each of said phenyl, phenoxy, phenyl-(lower alkyl) and phenyl-(lower alkoxy) groups being optionally substituted in one or more positions by a halogen atom and/or a lower alkyl and/or a lower alkoxy and/or a hydroxy and/or a lower alkylthio group; A¹⁰ is a divalent straight or branched (oxy/thio)-alkylene linkage containing from two to six carbon atoms and optionally one or two divalent oxygen and/or sulfur atom(s), provided that there are at least two carbon atoms between the divalent atom and the —NH— group and between the two divalent atoms; A²⁰ is a straight or branched alkylene chain containing from one to four carbon atoms; Z is a hydrogen atom or a lower alkyl group; and wherein in the definitions of R¹⁰, R²⁰, and Z, the term lower as applied to alkyl, alkoxy, or alkylthio groups or the alkyl, alkoxy, or alkylthio moieties of a group means an alkyl, alkoxy, or alkylthio group having 1 to 4 carbon atoms.

9. (canceled)

10. The method of claim 8, wherein the peripheral serotonin receptor antagonist is xylamidine.

11. The method of claim 10, wherein xylamidine is administered orally at a dose ranging from about 5 to about 200 mg per about 70 kg adult human or parenterally at a dose ranging from about 0.5 to about 20 mg per about 70 kg adult human.

12. The method of claim 11, wherein xylamidine is administered orally at a dose ranging from about 10 to about 100 mg per about 70 kg adult human or parenterally at a dose ranging from about 1 to about 10 mg per about 70 kg adult human.

13. (canceled)

14. (canceled)

15. (canceled)

16. (canceled)

17. The method of claim 8, wherein the peripheral serotonin receptor antagonist is BW501C67.

18. The method of claim 17, wherein BW501C67 is administered orally at a dose ranging from about 1 to about 100 mg per about 70 kg adult human or parenterally at a dose ranging from about 0.1 to about 10 mg per about 70 kg adult human.

19. The method of claim 18, wherein BW501C67 is administered orally at a dose ranging from about 2 to about 20 mg per about 70 kg adult human or parenterally at a dose ranging from about 0.2 to about 2 mg per about 70 kg adult human.

20. (canceled)

21. (canceled)

22. (canceled)

23. The method of claim 8, wherein the BW501C67 analog is 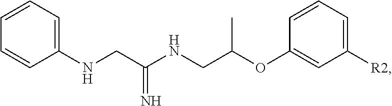
or a pharmaceutically acceptable acid addition salt thereof, wherein R2 is Br, F, H, benzyl, or alkyl containing 1-4 carbon atoms or is 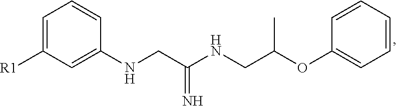
or a pharmaceutically acceptable acid addition salt thereof, wherein R1 is H, F, Cl, Br, Me, or OMe or is 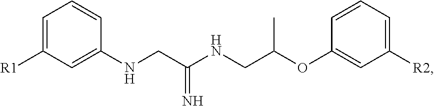
or a pharmaceutically acceptable acid addition salt thereof, wherein R1 is F, Cl, Br, Me, or OMe and R2 is Br, Cl, F, OMe, benzyl, or alkyl containing 1-4 carbon atoms.

24. (canceled)

25. (canceled)

26. (canceled)

27. (canceled)

28. The method of claim 1, wherein the 5-HT2A receptor agonist is selected from the group consisting of an ergoline, a tryptamine, a phenethylamine, and an amphetamine.

29. The method of claim 28, wherein the tryptamine is selected from the group consisting of psilocybin, psilocin, N,N-dimethyltryptamine (DMT), 5-methoxy-N,N-dimethyltryptamine (5-MeO-DMT), N-methyl-N-ethyltryptamine (MET), N-methyl-N-isopropyltryptamine (MIPT), N,N-diethyltryptamine (DET), N,N-diisopropyltryptamine (DIPT), N,N-dipropyltryptamine (DPT), N-cthyl-N-propyltryptamine (EPT), 5-methoxy-N-methyl-N-isopropyltryptamine (5-McO-MIPT), 5-methoxy-N,N-diisopropyltryptamine (5-MeO-DIPT), 5-methoxy-N-methyl-N-ethyltryptamine (5-MeO-MET), 5-methoxy-N,N-diethyltryptamine (5-McO-DET), N,N-diallyl-5-methoxytryptamine (5-McO-DALT), 4-hydroxy-N-methyl-N-ethyltryptamine (4-HO-MET), 4-hydroxy-N-methyl-N-isopropyltryptamine (4-HO-MIPT), 4-hydroxy-N,N-diisopropyltryptamine (4-HO-DIPT), 4-hydroxy-N,N-diethyltryptamine (4-HO-DET), 4-hydroxy-N,N-dipropyltryptamine (4-HO-DPT), 4-hydroxy-N-cthyl-N-propyltryptamine (4-HO-EPT), 4-acetoxy-N-methyl-N-ethyltryptamine (4-AcO-MET), 4-acetoxy-N-methyl-N-isopropyltryptamine (4-AcO-MIPT), 4-acetoxy-N,N-diisopropyltryptamine (4-AcO-DIPT), 4-acetoxy-N,N-diethyltryptamine (4-AcO-DET), 4-acetoxy-N,N-dipropyltryptamine (4-AcO-DPT), 4-acetoxy-N-ethyl-N-propyltryptamine (4-AcO-EPT), 4-acetoxy-N,N-dimethyltryptamine (4-AcO-DMT), alpha-methyltryptamine (AMT), alpha-cthyltryptamine (AET), and 5-methoxy-alpha-methyltryptamine (5-MeO-AMT); the ergoline is a lysergic acid amide selected from the group consisting of lysergic acid diethylamide (LSD), lysergic acid 2,4-dimethylazetidide (LSZ), 6-cthyl-6-nor-lysergic acid diethylamide (ETH-LAD), 6-propyl-6-nor-lysergic acid diethylamide (PRO-LAD), 1-acetyl-lysergic acid diethylamide (ALD-52), 1-propionyl-lysergic acid diethylamide (1P-LSD), 1-butyryl-lysergic acid diethylamide (1B-LSD), and 1-(cyclopropylmethanoyl)-lysergic acid diethylamide (lcP-LSD); the phenethylamine is selected from the group consisting of mescaline, escaline, proscaline, methallylescaline, allylescaline, 4-bromo-2,5-dimethoxypenethylamine (2C-B), 4-chloro-2,5-dimethoxypenethylamine (2C-C), 4-iodo-2,5-dimethoxypenethylamine (2C-I), 2,5-dimethoxy-4-methylphenethylamine (2C-D), 2-(4-Ethyl-2,5-dimethoxyphenyl) ethanamine (2C-E), 2-(2,5-Dimethoxy-4-propylphenyl) ethan-1-amine (2C-P), 2-[4-(Ethylsulfanyl)-2,5-dimethoxyphenyl]ethan-1-amine (2C-T-2), 2-[2,5-Dimethoxy-4-(propylsulfanyl)phenyl]ethan-1-amine (2C-T-7), 2-(4-iodo-2,5-dimethoxyphenyl)-N-[(2-methoxyphenyl)methyl]ethanamine (25I-NBOMc), 2-(4-bromo-2,5-dimethoxyphenyl)-N-[(2-methoxyphenyl)methyl]ethanamine (25B-NBOMe), 2-(4-chloro-2,5-dimethoxyphenyl)-N-[(2-methoxyphenyl) methyl]ethanamine (25C-NBOMc), 2-(4-methyl-2,5-dimethoxyphenyl)-N-[(2-methoxyphenyl)methyl]ethanamine (25D-NBOMe), 2-(4-ethyl-2,5-dimethoxyphenyl)-N-[(2-methoxyphenyl)methyl]ethanamine (25E-NBOMc), 2-(4-iodo-2,5-dimethoxyphenyl)-N-[(2hydroxyphenyl)methyl]ethanamine (25I-NBOH), 2-(4-bromo-2,5-dimethoxyphenyl)-N-[(2-hydroxyphenyl)methyl]ethanamine (25B-NBOH), 2- (4-chloro-2,5-dimethoxyphenyl)-N-[(2-hydroxyphenyl)methyl]ethanamine (25C-NBOH), 2-(4-methyl-2,5-dimethoxyphenyl)-N-[(2-hydroxyphenyl)methyl]ethanamine (25D-NBOH), 2-(4-ethyl-2,5-dimethoxyphenyl)-N-[(2-hydroxyphenyl)methyl]ethanamine (25E-NBOH), and 2-(4-cyano-2,5-dimethoxyphenyl)-N-[(2-hydroxyphenyl)methyl]ethanamine (25CN-NBOH); and the amphetamine is selected from the group consisting of 2,5-dimethoxy-4-methylamphetamine (DOM), 2,5-dimethoxy-4-bromoamphetamine (DOB), 2,5-dimethoxy-4-chloroamphetamine (DOC,) 2,5-dimethoxy-4-iodoamphetamine (DOI), 2,5-dimethoxy-4-ethylamphetamine (DOET), and 2,5-Dimethoxy-4-propylamphetamine (DOPR).

30. (canceled)

31. (canceled)

32. (canceled)

33. The method of claim 1, wherein the 5-HT2A receptor agonist is selected from the group consisting of psilocybin, 4-AcO-DMT, psilocin, DMT, 5-MeO-DMT, LSD, mescaline, 2C-B, 2C-E, 2C-T-2, 2C-T-7, and DOM.

34. The method of claim 1, wherein the 5-HT2A receptor agonist is selected from the group consisting of lysergic acid diethylamide (LSD), lysergic acid 2,4-dimethylazetidide (LSZ), 6-ethyl-6-nor-lysergic acid diethylamide (ETH-LAD), 6-propyl-6-nor-lysergic acid diethylamide (PRO-LAD), 1-acetyl-lysergic acid diethylamide (ALD-52), 1-propionyl-lysergic acid diethylamide (1P-LSD), 1-butyryl-lysergic acid diethylamide (1B-LSD), and 1-(cyclopropylmethanoyl)-lysergic acid diethylamide (1cP-LSD) administered at a dose ranging from bout 0.010 mg and to about 1.0 mg per about 70 kg human, wherein the 5-HT2A receptor agonist is administered parentally, orally, sublingually, buccally, or intranasally.

35. (canceled)

36. The method of claim 1, wherein the 5-HT2A receptor agonist is selected from the group consisting of 2,5-dimethoxy-4-methylamphetamine (DOM), 2,5-dimethoxy-4-bromoamphetamine (DOB), 2,5-dimethoxy-4-chloroamphetamine (DOC,) 2,5-dimethoxy-4-iodoamphetamine (DOI), 2,5-dimethoxy-4-ethylamphetamine (DOET), 2,5-Dimethoxy-4-propylamphetamine (DOPR), and 5-MeO-AMT administered at a dose ranging from about 0.10 mg to about 10 mg per about 70 kg human, wherein the 5-HT2A receptor agonist is administered parentally, orally, sublingually, buccally, or intranasally.

37. (canceled)

38. The method of claim 1, wherein the 5-HT2A receptor agonist is selected from the group consisting of psilocybin, psilocin, 5-MeO-MIPT, 5-MeO-DIPT, 5-MeO-MET, 5-MeO-DET, 5-MeO-DALT, 4-HO-MET, 4-HO-MIPT, 4-HO-DIPT, 4-HO-DET, 4-HO-EPT, 4-ACO-MET, 4-ACO-MIPT, 4-ACO-DIPT, 4-AcO-DET, 4-ACO-EPT, 4-AcO-DMT, alpha-methyltryptamine, 2C-B, 2C-C, 2C-1, 2C-E, 2C-P, 2C-T-2, and 2C-T-7administered at a dose ranging from about 1.0 mg to about 50 mg per about 70 kg adult human, wherein the 5-HT2A receptor agonist is administered parentally, orally, sublingually, buccally, or intranasally.

39. (canceled)

40. The method of claim 1, wherein the 5-HT2A receptor agonist is selected from the group consisting of MIPT, 2C-D, escaline, proscaline, methallylescaline, and allylescaline administered at a dose ranging from about 2.0 mg to about 100 mg per about 70 kg adult human, wherein the 5-HT2A receptor agonist is administered parentally, orally, sublingually, buccally, or intranasally.

41. (canceled)

42. The method of claim 1, wherein the 5-HT2A receptor agonist is selected from the group consisting of DET, DIPT, 4-HO-DPT, and 4-AcO-DPT administered at a dose ranging from about 5.0 mg to about 150 mg per about 70 kg adult human, wherein the 5-HT2A receptor agonist is administered parentally, orally, sublingually, buccally, or intranasally.

43. (canceled)

44. The method of claim 1, wherein the 5-HT2A receptor agonist is EPT or DPT administered at a dose ranging from about 20 to about 400 mg per about 70 kg adult human, wherein the 5-HT2A receptor agonist is administered parentally, orally, sublingually, buccally, or intranasally.

45. (canceled)

46. The method of claim 1, wherein the 5-HT2A receptor agonist is mescaline administered at a dose ranging from about 50 mg to about 1,000 mg per about 70 kg adult human, wherein the 5-HT2A receptor agonist is administered parentally, orally, sublingually, buccally, or intranasally.

47. (canceled)

48. (canceled)

49. (canceled)

50. (canceled)

51. (canceled)

52. The method of claim 1, wherein the 5-HT2A receptor agonist is a tryptamine which is 5-MeO-DMT and is administered at a dose ranging from about 1.0 mg to about 30 mg per about 70 kg adult human and the 5-HT2A receptor agonist is administered by inhalation as a vapor or parenterally and wherein the method optionally additionally comprises administering a monoamine oxidase inhibitor.

53. (canceled)

54. (canceled)

55. The method of claim 1, wherein the 5-HT2A receptor agonist is a tryptamine selected from the group consisting of DMT and MET, wherein the 5-HT2A receptor agonist is administered at a dose ranging from about 5.0 to about 100 mg per about 70 kg adult human, wherein the 5-HT2A receptor agonist is administered by inhalation as a vapor or parenterally, and wherein the method optionally additionally comprises administering a monoamine oxidase inhibitor.

56. (canceled)

57. (canceled)

58. (canceled)

59. (canceled)

60. (canceled)

61. The method of claim 52, wherein when a monoamine oxidase inhibitor is administered, then the tryptamine is orally administered.

62. The method of claim 1, wherein the 5-HT2A receptor agonist is selected from the group consisting of 25I-NBOMe, 25B-NBOMe, 25C-NBOMe, 25D-NBOMe, and 25E-NBOMe administered at a dose ranging from about 0.05 mg to about 2.0 mg per about 70 kg adult human, wherein the 5-HT2A receptor agonist is administered parenterally, sublingually, or buccally.

63. (canceled)

64. The method of claim lany one of claims 1, wherein the 5-HT2A receptor agonist is selected from the group consisting of 25I-NBOH, 25B-NBOH, 25C-NBOH, 25D-NBOH, 25E-NBOH, and 25CN-NBOH administered at a dose ranging from about 0.05 mg to about 3.0 mg per about 70 kg adult human, wherein the 5-HT2A receptor agonist is administered parenterally, sublingually, or buccally.

65. (canceled)

66. (canceled)

67. (canceled)

68. The method of claim 1, wherein the 5-HT2A receptor agonist is represented by 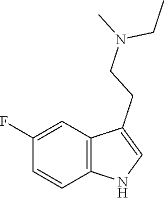 [SMILES: CCN(C)CCc1c[nH]c2ccc(F)cc12]
or a pharmaceutically acceptable salt thereof or 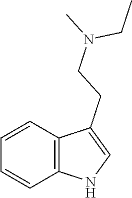
or a pharmaceutically acceptable salt thereof.

69. (canceled)

70. The method of claim 33, wherein the 5-HT2A receptor agonist is administered at a dose and by a route as follows:
psilocybin, 4-AcO-DMT, or psilocin—about 10-50 mg, PO
DMT—about 10-50 mg, vaporized (inhaled) or IV
5-MeO-DMT—about 5-25 mg, vaporized (inhaled) or IV
LSD—about 50-300 μg, PO
mescaline—about 100-500 mg, PO
2C-B, 2C-E, 2C-T-2, or 2C-T-7—about 5-30 mg, PO
DOM—about 2-10 mg, PO, all doses per about 70 kg adult human.

71. The method of claim 1, wherein the psychiatric disorder is a mood disorder.

72. The method of claim 71, wherein the mood disorder is selected from the group consisting of a depressive disorder and a bipolar disorder.

73. (canceled)

74. The method of claim 71, wherein the mood disorder is a treatment-resistant depressive disorder.

75. The method of claim 71, wherein the mood disorder is selected from the group consisting of major depressive disorder, persistent depressive disorder, postpartum depression, premenstrual dysphoric disorder, seasonal affective disorder, psychotic depression, disruptive mood dysregulation disorder, substance/medication-induced depressive disorder, depressive disorder due to another medical condition, a substance-related disorder, a substance-use disorder, an anxiety disorder, obsessive-compulsive and related disorders, trauma-and stressor-related disorders, feeding and eating disorders, borderline personality disorder, attention-deficit/hyperactivity disorder, and autism spectrum disorder.

76. (canceled)

77. (canceled)

78. (canceled)

79. (canceled)

80. The method of claim 55, wherein when a monoamine oxidase inhibitor is administered, then the tryptamine is orally administered.
